(19) United States
(12) Patent Application Publication
(10) Pub. No.: US 2023/0109946 A1
(43) Pub. Date: Apr. 13, 2023

(54) APPARATUS FOR COMPUTER GENERATED DIALOGUE AND TASK-SPECIFIC NESTED FILE ARCHITECTURE THEREOF

(71) Applicant: Twill, Inc., New York, NY (US)

(72) Inventors: Jared Minkel, Kingstown, RI (US); Janis Martman, British Columbia (CA); Chris Wasden, Park City, UT (US); Ran Zilca, Ra'anana (IL); Tomer Ben-Kiki, New York, NY (US)

(73) Assignee: Twill, Inc., New York, NY (US)

(21) Appl. No.: 17/878,901

(22) Filed: Aug. 1, 2022

Related U.S. Application Data
(60) Provisional application No. 63/254,623, filed on Oct. 12, 2021.

(51) Int. Cl. G16H 20/70

(52) U.S. Cl. CPC G16H 20/70

(57) ABSTRACT

The present disclosure relates to digital devices adapted to increase the efficacy of a computer-implemented migraine treatment plan. In some embodiments, an apparatus generates an interactive session comprising a plurality of tracks and sets a need set according to a baseline level. The need set may comprise a selection of one or more tracks, wherein the selection is a function of the baseline level and/or a variable level. The interactive session may utilize a three-tiered architecture interactive dialogue module comprising a master file, a plurality of skeleton files, and a plurality of skin sets, wherein each of the plurality of skin sets is nested within one of the plurality of skeleton files and the plurality of skeleton files are nested within the master file. Accordingly, the tracks, the activities, and the tasks, may each utilize the master file, the skeleton files, and the skin sets, respectively.

### FIELD OF THE INVENTION

The present invention relates to apparatuses for computer implemented digital behavioral therapy for the treatment of conditions, symptoms, side-effects and comorbidities directly and indirectly related to migraine. Specifically, the present invention relates to an adaptable user interface comprising tiered speech architecture.

### INTRODUCTION

Currently, there is an expansion of all facets of the medical industry into the digital space, including, but not limited to medical treatments and therapies. For example, many therapies that once required a one-on-one session with a physician may now be supplemented, or completely administered, with digital therapies. However, such currently available digital therapies may be insufficient in treating the physical and mental conditions of many patients. In order to treat many conditions, the patient must be continuously evaluated and treatments must be frequently readapted as a function of the patient's progress.

Specifically, migraine is a complex, common neurological condition characterized by severe, episodic attacks of headache and associated features such as nausea, vomiting, sensitivity to light, sound or movement. Chronic migraine is when a migraine occurs 15 or more days per month. However, symptoms in chronic migraine often change frequently as may the severity of the pain. Primarily due to the high frequency of chronic migraine, it has a particularly debilitating impact on the patient's quality of life and has the potential to be a primary feature of the patient's life. Sufferers of chronic migraine have high incidence of depression, anxiety, employment issues and lower socioeconomic status than the general public.

Accordingly, in order to adequately treat migraine, a condition that rapidly and drastically manifests various symptoms, the patient's treatment must be responsive to the instant status of their condition. However, current digital therapies are insufficient in providing direction to patient's in a rapid and personalized manner. Specifically, the architecture of current digital therapies are incapable of processing and delivering meaningful communications to a patient.

Thus, it would be desirable to provide a system for digital therapeutics capable of delivering rapid and personalized communications to a patient. It would be further desirable to provide a system configured to interpret patient behavior and alter the digital therapeutic tracks accordingly. It would be yet further desirable to provide a digital therapeutic system comprising a tiered speech architecture system.

### SUMMARY OF AN EMBODIMENT

Disclosed herein are devices and methods involving digital therapeutics for treating migraine, symptoms associated with migraine, symptoms associated with side-effects of migraine medications, and comorbidities of migraine. A digital device for use in such treatment includes a display, an input device, one or more processors, networking interfaces and memory storing one or more software programs configured to be executed by the one or more processors.

The invention of the present disclosure may be an apparatus configured to increase the efficacy of a computer-implemented migraine treatment plan and adherence to said treatment plan, the apparatus comprising at least one processor, at least one display, at least one memory comprising computer-executable instructions which, when executed by the at least one processor, cause the apparatus to generate, via the at least one processor, an interactive session comprising a plurality of tracks, each of the plurality of tracks comprising one or more activities; and store, via the at least one memory, a baseline user metric (for example, a baseline user happiness level) and a variable user metric (for example, a variable user happiness level), wherein the variable user happiness level is updateable based on progress of the plurality of tracks. The computer-executable instructions may further cause the apparatus to generate, via the at least one processor, an assessment configured to receive a initial set of user information; determine, via the at least one processor, the baseline user happiness level according to the assessment and the initial set of user information; and set, via the at least one processor, a need set according to at least the baseline user happiness level and the variable happiness level, the need set comprising a selection of one or more of the plurality of tracks, wherein the selection of one or more of the plurality of tracks is configured to increase the variable happiness level.

In an aspect, the plurality of tracks comprises an interactive dialogue module, wherein, when engaged in the interactive dialogue module, the apparatus is configured to receive a user input. In a further aspect, the interactive dialogue module comprises a three-tier architecture comprising a master file, a plurality of skeleton files, and a plurality of skin sets, wherein each of the plurality of skin sets is nested within one of the plurality of skeleton files and the plurality of skeleton files are nested within the master file.

In an embodiment, each of the plurality of activities comprise one or more tasks, wherein the plurality of tracks correspond to the master file, wherein the plurality of activities correspond to the plurality of skeleton files, and wherein the one or more tasks correspond to plurality of skin sets.

In yet a further aspect, the interactive dialogue module comprises a dialogue interface, the computer-executable instructions which, when executed by the at least one device processor, further cause the apparatus to identify, via the at least one processor, a selected skeleton file and a selected skin set according to an instant task, wherein the instant task is the one of the one or more tasks engaged on the apparatus; generate, via the at least one processor, a message according to at least the selected skeleton file; and display, via the apparatus, the message. Each of the one or more tasks and each of the plurality of activities may comprise one of a plurality of difficulty levels, wherein each of the one or more tasks and each of the plurality of activities may be unlocked according to a corresponding difficulty level relative to the variable user happiness level.

In another aspect, the computer-executable instructions which, when executed by the at least one device processor, further cause the apparatus to associate, via the at least one processor, one of a plurality of badges to a user profile, wherein each of the plurality of badges correspond to completion of a given task, activity, or track.

Additional aspects related to this disclosure are set forth, in part, in the description which follows, and, in part, will be obvious from the description, or may be learned by practice of this disclosure.

It is to be understood that both the forgoing and the following descriptions are exemplary and explanatory only and are not intended to limit the claimed disclosure or application thereof in any manner whatsoever.

### BRIEF DESCRIPTION OF THE FIGURES

Objects, aspects, features, and advantages of embodiments disclosed herein will become more fully apparent from the following detailed description, the appended claims, and the accompanying drawing figures in which like reference numerals identify similar or identical elements. Reference numerals that are introduced in the specification in association with a drawing figure may be repeated in one or more subsequent figures without additional description in the specification in order to provide context for other features, and not every element may be labeled in every figure. The drawing figures are not necessarily to scale, emphasis instead being placed upon illustrating embodiments, principles and concepts. The drawings are not intended to limit the scope of the claims included herewith.

The present disclosure will become more fully understood from the detailed description and the accompanying drawings, wherein:

FIG. 1 shows an example of a client-server based distributed communication system that can be used to implement an online service for enhancing mental health of users and a dialogue management system for the online service;

FIG. 2 shows an example of a client device of the distributed communication system of FIG. 1;

FIG. 3 shows an example of a block diagram of a server of the distributed communication system of FIG. 1;

FIG. 4 shows an example of a block diagram of the online service;

FIG. 5A shows an example of a block diagram of the dialogue management system;

FIG. 5B shows an example of a dialogue box (also called a dialog box) including a dialogue between the online service of FIG. 4 and a user of the online service using the dialogue management system of FIG. 5A;

FIG. 6 shows an example of a flowchart of a method of conducting a dialogue between the online service of FIG. 4 and a user of the online service using the dialogue management system of FIG. 5A;

FIG. 7 shows an example of a flowchart of a method of creating a master dialogue file for conducting a dialogue between the online service of FIG. 4 and a user of the online service using the dialogue management system of FIG. 5A;

FIG. 8 shows an example of a flowchart of a method of creating a skeleton file for conducting a dialogue between the online service of FIG. 4 and a user of the online service using the dialogue management system of FIG. 5A;

FIG. 9 shows an example of a flowchart of a method of creating a skin file for conducting a dialogue between the online service of FIG. 4 and a user of the online service using the dialogue management system of FIG. 5A;

FIGS. 10A-10N show a table including examples of tracks and activities offered by the online service to the users of the online service of FIG. 4 for improving mental health of the users;

FIGS. 11A-11C show a table including an example of a track, activities of the track, and tasks of the activities offered by the online service for FIG. 4 for improving mental health of the users; and

FIG. 12 is an exemplary flowchart explaining proactive triaging feature of the present invention.

In the drawings, reference numbers may be reused to identify similar and/or identical elements.

### DETAILED DESCRIPTION

Some aspects and embodiments of the disclosed invention will be described more fully with reference to the accompanying drawings. This disclosed invention may be embodied in many different forms and should not be construed as limited to the aspects and embodiments set forth herein.

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

### Computing System for Implementing a Digital Therapeutic

The present invention includes and requires an interactive computing system that provides an environment in which a human user interacts with the computer for the purpose of achieving one or more clinical benefits to the user. The clinical benefit to the user can be relatively direct in nature, such as decreasing the level of depression or decreasing the level of anxiety of the user. Clinical benefits of a somewhat indirect nature may also be achieved. For example, if depression and/or anxiety are significant comorbidities that may amplify the impact of symptoms of another disease, then managing depression and/or anxiety will result in treating the other disease symptoms.

A digital therapeutic regimen may also have the benefit, which may be classified as direct or indirect, of increasing medication compliance and adherence. Medication compliance is defined as how well a patient follows the directions written on a prescription. Medication adherence is related to compliance but involves the level of motivation a patient has in sticking to a therapeutic regimen. Adherence is often impacted by social and environmental influences. Difficult side effects of a drug will have a tendency to negatively impact adherence and compliance. This is because the prevalence and severity of side effects varies among patients and, just as important, patients possess varying levels of motivation regarding sticking to their therapeutic regimen. Whereas minor side effects require minimal motivation to adhere to a regimen, major side effects will require greatly increased motivational basis. Severe side effects may also have an impact on a patient's ability to properly weigh the costs versus the benefits of a given therapeutic regimen. As an extreme example, the severe side effects of some chemotherapy regimens result in poor adherence and compliance in spite of the huge benefits said regimen has upon the patient.

A more prosaic example of the impact of side effects on adherence is a treatment for migraine that results in constipation. Although it would be hoped that very few patients would tend toward non-compliance with moderate constipation, i.e., choose more migraines to avoid the constipation side effect, human psychology simply does not work this way. Whatever the mental process of a given patient, e.g., choosing the devil you know versus the devil you do not know, there are many examples of drugs having poor adherence and compliance in spite of the relative benefit of the treatment being substantial and the side effect, i.e., cost, being low or moderate. There are a number of potential explanations for this counterintuitive result but, ultimately, much will depend on the individual psychology of a particular patient. Some patients may have an increase in depression and/or anxiety based on a side effect and this increase may, in some portion of patients suffering this side effect, lower the patient's adherence by having an outsized impact on their cost/benefit calculation.

Other psychological factors may have a significant impact on a patient's compliance or, perhaps, be utilized in increasing their adherence. For example, mindfulness may be used to encourage the patient to fully appreciate the costs versus benefits of adhering to a medication regimen. By having a positive impact on the patient's psychology, including increasing their mindfulness, the patient is better prepared for dealing with side effects.

In general, and as described in greater detail herein, the computing system is configured to provide and engage the user in a set of activities and tasks particularly designed and selected for that user to increase the user's level of happiness and lower their level of anxiety. The system may also be configured to address symptoms of migraine as well as side-effects associated with various migraine treatment regimens.

In accordance with the present invention, the computing system dynamically responds to the user's actions and feedback, which result from the user's partial or full performance of certain activities and tasks, and such dynamic responding by the computing system entails interaction that includes demonstration of simulated human emotion and/or human cognitive skill, such as empathy. As will be further described, interaction that includes demonstration of simulated human emotion and/or human cognitive skill results in a more personal and in-context environment with the user, mimicking a human-to-human conversation that, in turn, resulting in a manner of guiding the user that leads to achieving the desired goal.

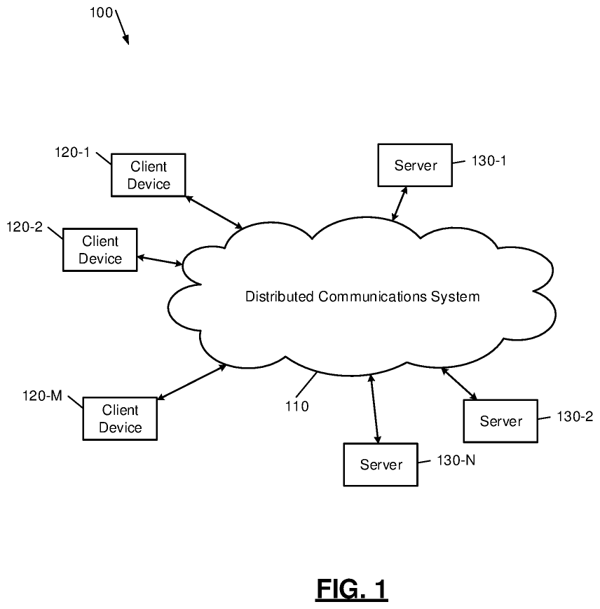

FIG. 1 shows a simplified example of a distributed app 200. The distributed app 200 includes a distributed communications system 110, one or more client devices 120-1, 120-2, etc., collectively, client devices 120 and one or more servers 130-1, 130-2, etc., collectively, servers 130. The distributed communications system 110 may include a local area network (LAN), a wide area network (WAN) such as the Internet, or other type of network. The client devices 120 and the servers 130 may be located at different geographical locations and communicate with each other via the distributed communications system 110. The client devices 120 and the servers 130 connect to the distributed communications system 110 using wireless and/or wired connections. The client devices 120 may include smartphones, personal digital assistants (PDAs), tablets, laptop computers, personal computers (PCs), etc. The servers 130 may provide multiple services to the client devices 120. For example, the servers 130 may execute software applications developed by one or more vendors. The servers 130 may host multiple databases that are relied on by the software applications in providing services to users of the client devices 120. For example, one or more of the servers 130 execute an application that implements the online service including the dialogue management system of the present disclosure.

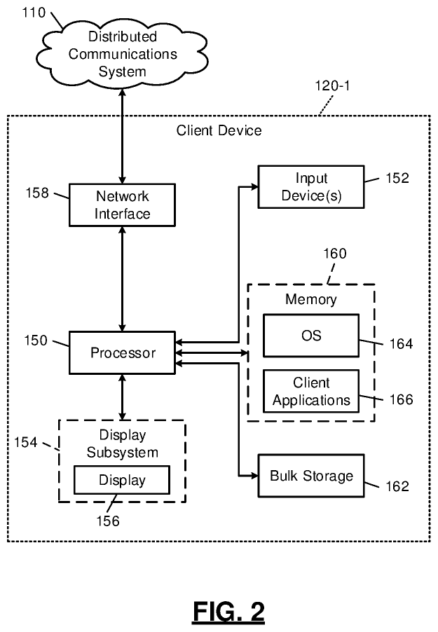

FIG. 2 shows a simplified example of the client device 120-1. The client device 120-1 may typically include a central processing unit (CPU) or processor 150, one or more input devices 152, e.g., a keypad, touchpad, mouse, touchscreen, etc.), a display subsystem 154 including a display 156, a network interface 158, memory 160, and bulk storage 162. The network interface 158 connects the client device 120-1 to the distributed app 200 via the distributed communications system 110. For example, the network interface 158 may include a wired interface (for example, an Ethernet interface) and/or a wireless interface (for example, a Wi-Fi, Bluetooth, near field communication (NFC), or other wireless interface). The memory 160 may include volatile or nonvolatile memory, cache, or other type of memory. The bulk storage 162 may include flash memory, a magnetic hard disk drive (HDD), and other bulk storage devices. The processor 150 of the client device 120-1 executes an operating system (OS) 164 and one or more client applications 166. The client applications 166 include an application that accesses the servers 130 via the distributed communications system 110. The client applications 166 include an application that accesses the online service including the dialogue management system executed by one or more of the servers 130.

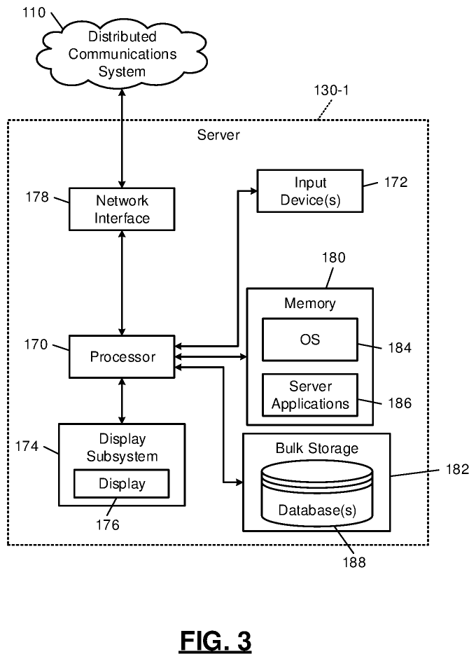

FIG. 3 shows a simplified example of the server 130-1. The server 130-1 typically includes one or more CPUs or processors 170, a network interface 178, memory 180, and bulk storage 182. In some implementations, the server 130-1 may be a general-purpose server and include one or more input devices 172, e.g., a keypad, touchpad, mouse, and so on) and a display subsystem 174 including a display 176. The network interface 178 connects the server 130-1 to the distributed communications system 110. For example, the network interface 178 may include a wired interface, e.g., an Ethernet interface) and/or a wireless interface, e.g., a Wi-Fi, Bluetooth, near field communication (NFC), or other wireless interface). The memory 180 may include volatile or nonvolatile memory, cache, or other type of memory. The bulk storage 182 may include flash memory, one or more magnetic hard disk drives (HDDs), or other bulk storage devices. The processor 170 of the server 130-1 executes an operating system (OS) 184 and one or more server applications 186, which may be housed in a virtual machine hypervisor or containerized architecture, which include the online service and the dialogue management system of the present disclosure. The term “application” or “app” is used extensively herein and includes the online service and similar related concepts. The bulk storage 182 may store one or more databases 188 that store data structures used by the server applications 186 to perform respective functions.

The online service or app is a science-based online service and social community for engaging, learning and training the skills of happiness and related skills for improving mental health attributes. The app can be offered through a variety of computing devices including smartphones, tablets, laptops, etc. The app is based on a framework developed by psychologists and researchers in areas such as positive psychology and neuroscience. The app assists users in the development of skills such as, for example, Savor, Thank, Aspire, Give, and Empathize (or S.T.A.G.E.™). The app includes an additional happiness skill called Revive that is concerned with physical wellness. Throughout the present disclosure, references are made to the STAGE skills for convenience only, and such references should be understood to include the sixth Revise skill. Each skill may be developed using various activities, ordered in increasing skill level, that gradually unlock as the user progresses in building that skill. Users of the app may be given a range of activities from the STAGE skills, from reflective blogging and science-based games and quizzes, to real-life tasks that the users are asked to perform and report back on. Each activity is backed by scientific studies that may be directly accessible by the user via links provided by the app in the recommended activities.

The activities may be offered to users in several ways. One such offering described below focuses on “tracks” that include sets of activities programmed to address a specific life situation or goal, e.g., “Cope better with stress;” “Enjoy parenting more”, etc. Beginning the app, users may complete self-assessments that give them their initial happiness level as well as an initial recommended track. Alternatively, as described in detail below, a particular order of tracks designed to address a particular need set of a user may be implemented. The term ‘need set’ may involve a condition, e.g., migraine, suffered by the user and the symptoms, side effects and comorbidities associated therewith. Tracks may be organized into modules of several tracks with modules also being determined based on need set. When users finish a track part, users may win, for example, a badge that represents their level of activity in that track part.

As users perform their activities, users may create activity posts that are saved in their personal profile and build up a ‘digital happiness wallet’ they can reflect on. Posts may include the type of activity performed by the user, any text and images the user added, other people involved, if any, as well as the time and location for the post. When the activity is a conversation performed with the dialogue management system, a post may include a summary of record of the conversation. Posts also may appear on various activity feeds on the service, which allows other users to read, draw inspiration from, and offer encouragement in the form of comments and likes. Users may also follow activities posted by other users they find interesting if those users allow themselves to be followed or mark their post ‘public.’ Periodically, the app may make suggestions for users to follow other users whose profiles match in terms of demographics and psychographics, as well as level of activity on the site and other criteria.

Periodic, scientifically-designed assessments are an important part of the app and may track a number of relevant parameters related to conditions, symptoms, side-effects and comorbidities suffered by the user. These parameters may be compared to past levels. Over time, the online service may build graphs for the user, comprising of activities, people, places, and things correlated with the impact they had on the parameters being tracked for user. This information may be used to optimize the user experience and the activities the app suggests.

Benefits provided by the app include: clarity, e.g., 5 skills, level progression), integrated self-assessments, e.g., provides self-insights, recommends tracks & activities), progress measurement, e.g., periodic happiness measurements allow the users to monitor their progress), guided experience, e.g., four week track experience optimizes habit formation, enables continued focus on a specific topic, e.g., parenting, stress)), flexible, e.g., track structure allows the users to pick the activities and tasks they prefer from a wider selection of options), personalized, e.g., activity recommendations are based on past user behavior and preference), integrated social experience, e.g., users share and follow, like and comment on other users' posts), increasingly challenging, e.g., as the users progress, tracks require increased number of activities and higher level of challenge), entertaining, e.g., variety of activity types, track content), extendible in several dimensions, e.g., content: new tracks and track content (tasks, quizzes, polls etc.), activity types: adding new games and activity types, framework: adding new skills), and multi-screen, e.g., web, mobile accessibility).

The app employs a science-to-action framework, provides sustained guidance, allows users to grow visual environments by interacting with them directly, provides contextual social interaction, e.g., users socialize around contextual activity posts prescribed to others, provides activity variety, e.g., real-life, reflective and gaming activities, provides measure-act-measure loop, e.g., allowing users to track their progress as they go, and provides an efficient and versatile dialogue management system that uses a 3-tier architecture to facilitate dialogues about multiple activities performed by multiple users using the least amount of data structures.

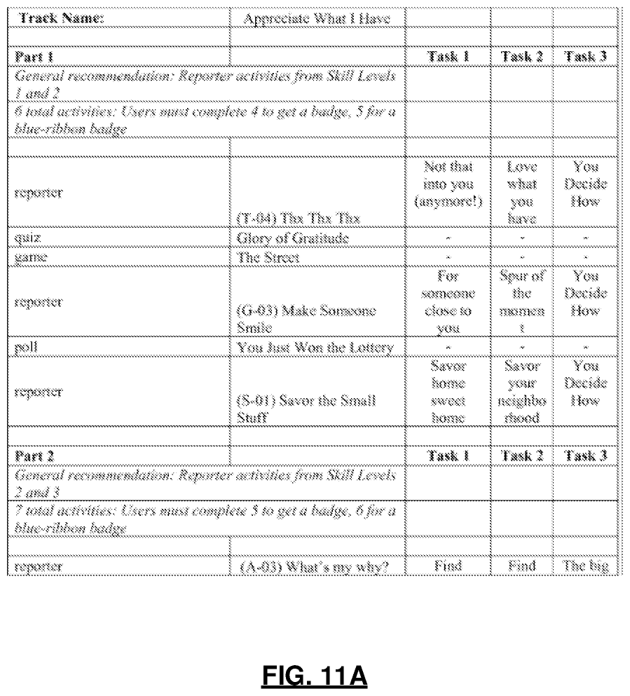

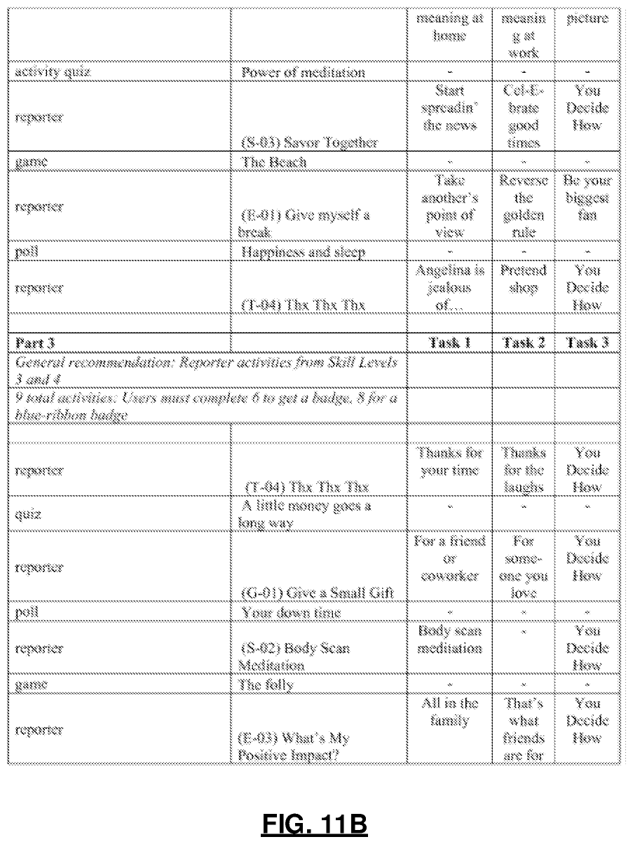

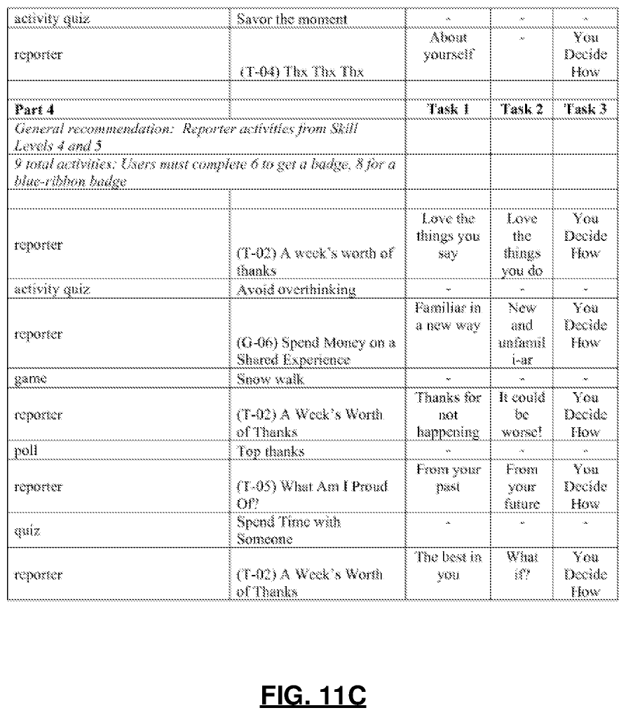

The tracks, modules, activities, and tasks offered by the app are now described in further detail to enhance understanding of the dialogue management system. Tracks are sets of activities that are programmed together to address specific life situations, goals, or concerns that users have. Each track is composed of multiple parts (described below; also see FIGS. 11A-11C). The number of activities and their level of difficulty may be set to increase as the user progresses.

The following are examples of rules that may be used to govern the tracks. Users may be afforded a set time period during which to complete a track part and thus earn badges. Badges may be regular or honors badge, depending on the number of activities they completed. Users may be allowed to extend beyond the set time period and still win the regular badge. If a user reaches the regular badge threshold the user is allowed to ‘win’ the regular badge and move to the next part, or continue for the honors badge. This allows users to skip the remaining activities and win the regular badge if they prefer.

At any time, multiple activities may be available for the user to perform with one or more being ‘queue-locked,’ which means that if the user performs an available activity, it will make the ‘queue-locked’ activity become available. Each day, for example, three time-locked activities become ‘queue-locked,’ and queue-locked activities become available up to a limit of four available activities. This limit of four available activities is intended to avoid showing the users too many available activities when they next log in.

Every activity a user completes creates a post that may be added to the user's profile. Users can mark their posts private, i.e., only visible to them and not visible to others) or viewable to other people (people who follow them and people doing the track in group mode with them). As part of social interaction, users can view the shared posts of other people who are following the track and can like or comment on them or follow the authors of those posts. Users can like and comment on posts to encourage each other and discuss their contents.

‘Career and money’ tracks include activities directed to the following aspects: appreciate what I have (currently available), reduce on-the-job stress, get energized about my job, stay upbeat while out of work, balance work and home life, and control my spending habits.

‘Family and kids’ tracks include activities directed to the following aspects: enjoy parenting more, better cope with new parenthood, better adjust to becoming an empty nester, forgive and forget feud (with a family member), and better cope with the stresses related to my aging parents.

‘Leisure and friends’ tracks include activities directed to the following aspects: be more socially connected, talkers and listeners, explore the art in happiness, find more “me” time, and be a better friend.

‘Love and intimacy’ tracks include activities directed to the following aspects: feel more loved by my partner, feel and be more devoted to my spouse, fight less and love more in my relationship, get over a broken heart, and feel hopeful to start dating after divorce.

‘Mind and body’ tracks include activities directed to the following aspects: cope better with stress, nurture my body and soul, come to terms with getting older, feel healthier, be more optimistic about my potential, and find more purpose and meaning in my life.

Each part of a track may include a balanced mix of ‘reporter’ activities and ‘light’ activities. The reporter activities may gradually increase in difficulty as a user progress through each of the four parts. Light activities may include: games, e.g., mini games, such as hidden object “mindfulness” game, training the user on a specific happiness skill), quizzes, e.g., multiple-choice or true/false questions about a happiness topic), activity quizzes, e.g., users read a science paragraph about an activity and are quizzed with multiple-choice questions at the end), and polls, e.g., polling users' opinion about a related topic and showing them community's vote breakdown). Reporter activities fall into two categories: “essay” or “do” activity, which asks users to reflect on a subject and make a log entry, e.g., reflective microblogging: users are asked to reflect on a topic and write down their thoughts, like what they are grateful for, what they look forward to, taking another person's perspective, etc.); and “plan-do” activity, which asks user to plan and perform an action in the real world, then come back and report on how it went, e.g., write about his/her experience in a savoring exercise)). The conversational activities, i.e., the conversations performed with the dialogue management system) are different than reporter activities.

A mix of about 50% “reporter” activities and 50% “light” activities may be used in each track part to avoid overwhelming the user. The online service allows for an activity to appear more than once in a track if it's a crucial activity for the track theme and there are new/different suggested tasks for each use. The number of activities per track part is flexible.

For example, a 7-day sequence of every track part includes a narrative purpose and a feel as if it has a beginning, middle, and an end that gives the user a sense of accomplishment. In the first days of a track part, the activities jump-start a key positive emotion the user will need for subsequent activities or asks the user to try something new, intriguing, fun, or funny—which rattles the user out of her funk and gets her in a good mood for what's next. In the middle of a track part, the activities build on (or complement) previous ones. An activity may be introduced that needs some extra thought or action. By day 4 or 5, the user feels a little more committed or motivated and willing to take on slightly more demanding activities. In the end, on the last day of a track part, users want something that's fun, easy or inspiring. Accordingly, unfamiliar/demanding tasks are avoided. The users anticipate a feeling of accomplishment but is intrigued enough to commit to the next part of their track.

The goal of the tracks is to create an appealing balance between activities that can be completed immediately by writing after a few minutes of reflection versus activities that require action (and in some cases, pre-planning) before reporting on how it went. In general, easier (levels 1 and 2) activities are programmed towards the beginning of a track (parts 1 and 2), and as a user progresses to the later parts of a track, the activities become more difficult (levels 4 and 5 activities), but this is not required. Users are awarded badges based on how many activities they complete in each part of a track. The online service offers special badges for each part of a track.

Users interacting with the app may start off at level-1 in all skills. As they complete activities they may progress in each skill from level-1 to level-2, etc. New activities, self-assessments, and other options may unlock as the user reaches a higher level. For each skill, the app offers relevant, science based activities that train the user in an entertaining way. As the users level up in a skill, they unlock new activities (level 1 to level 5 activities may be made available in each skill). Each activity provides the user with several alternatives for completing the activity (“Suggested Tasks”) to pick from. Users can view an explanation of “why it works”: a short summary of the science behind that activity, complete with links to the actual study this activity is based on.

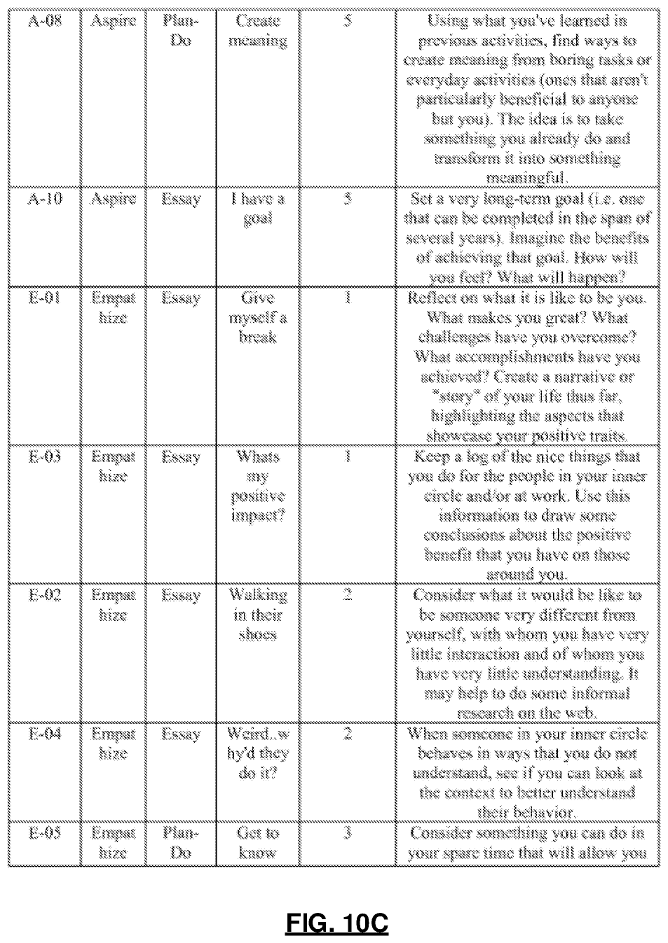

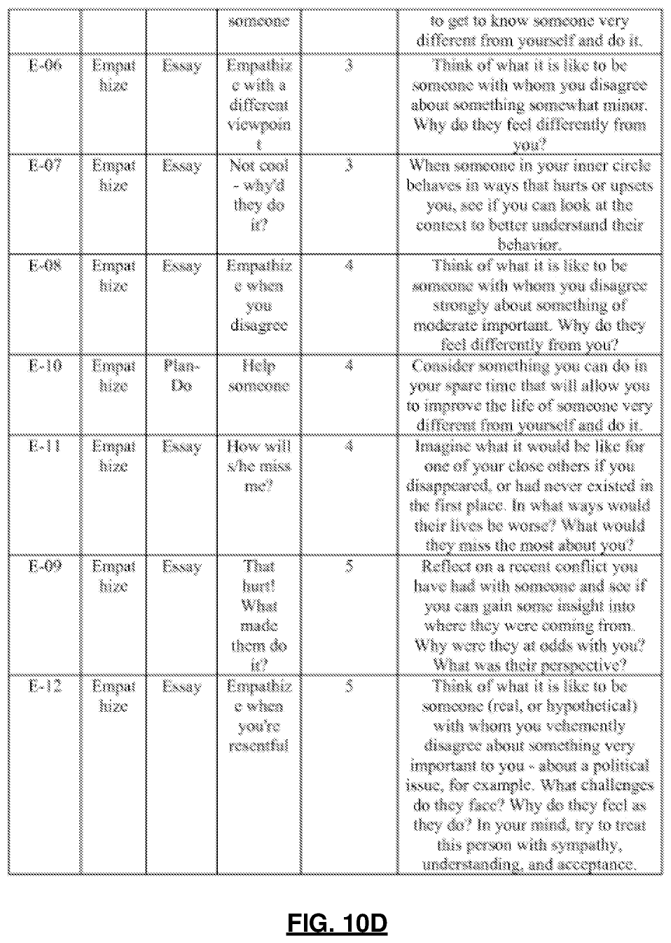

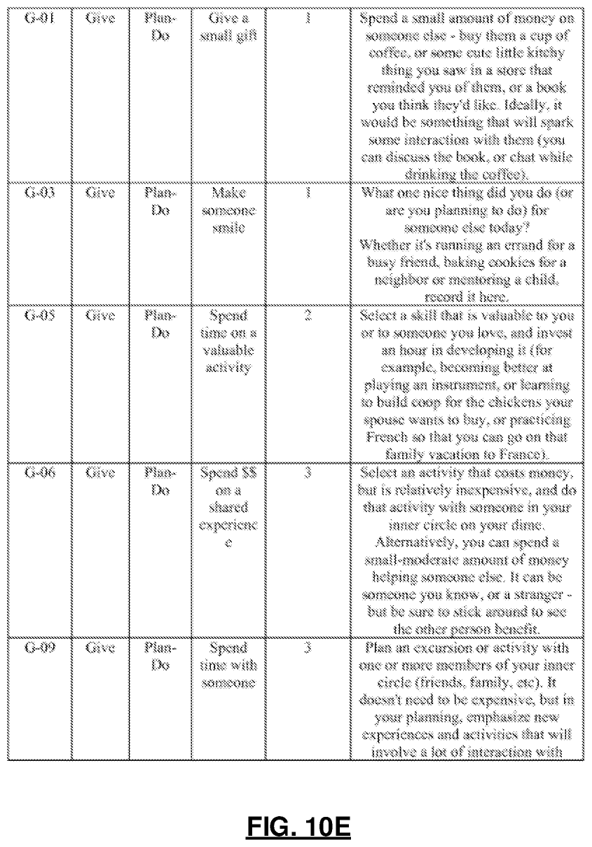

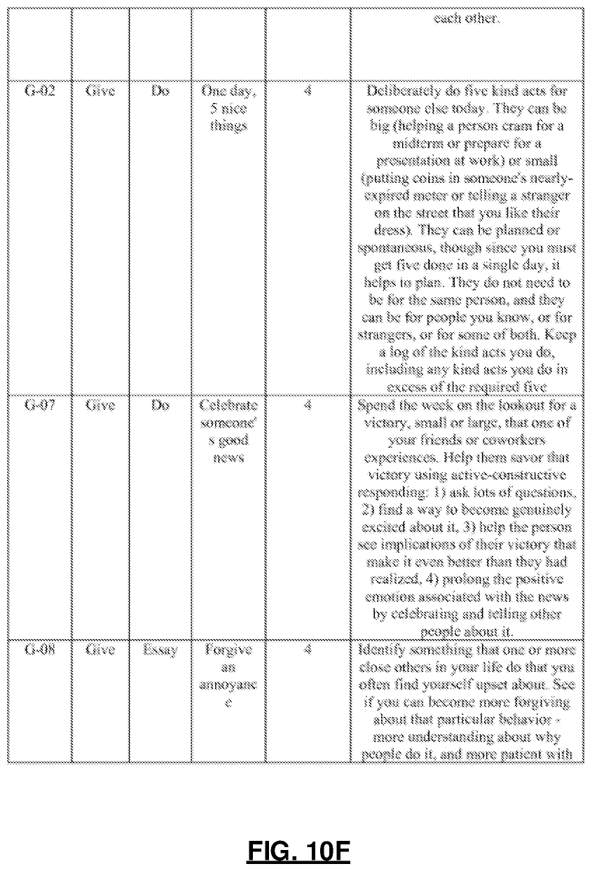

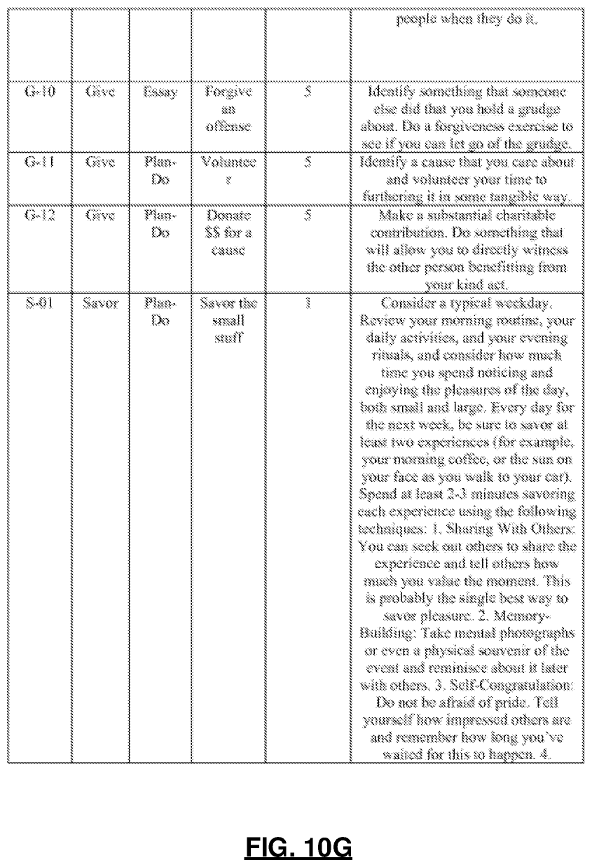

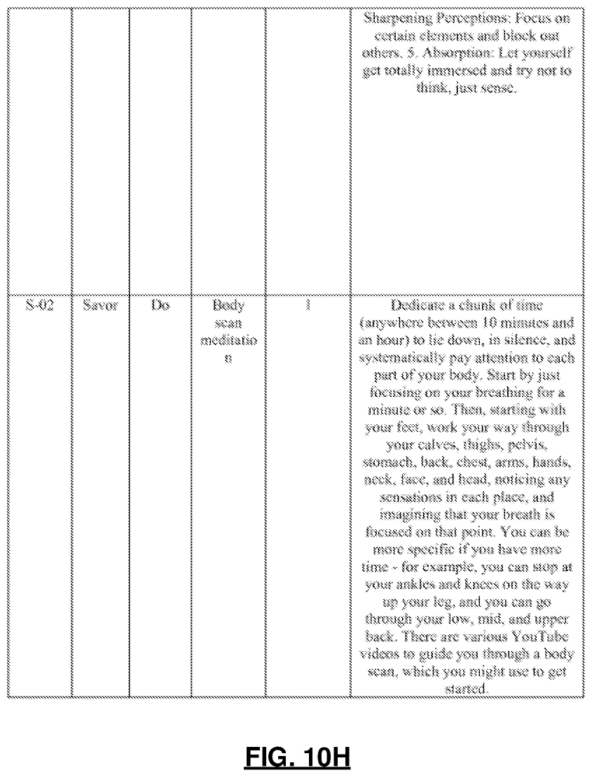

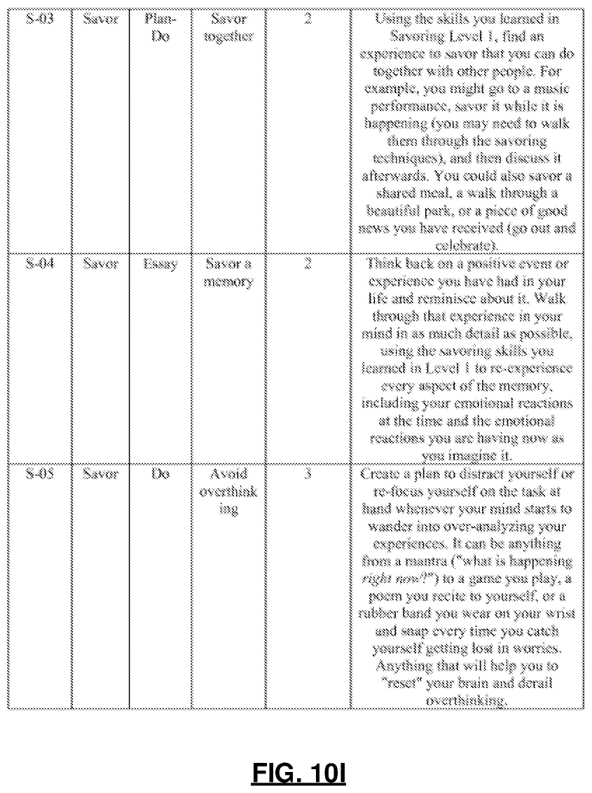

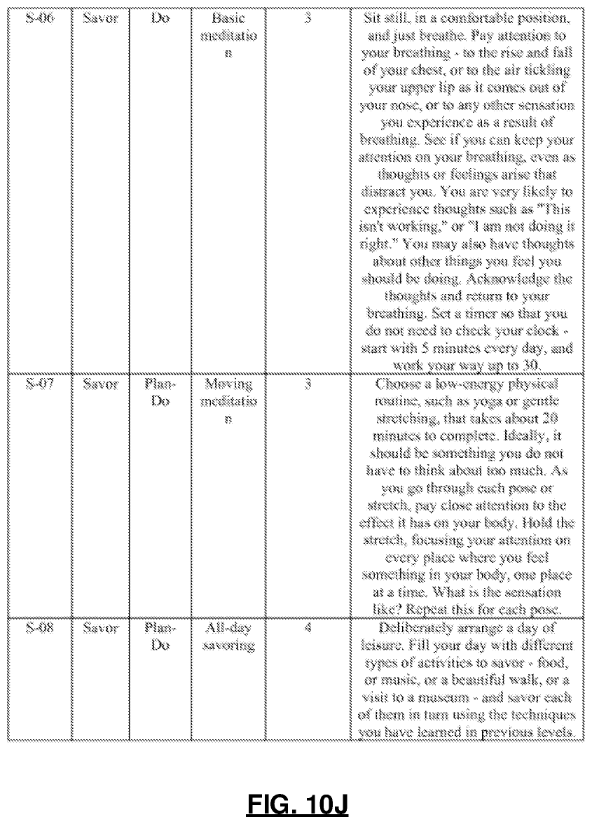

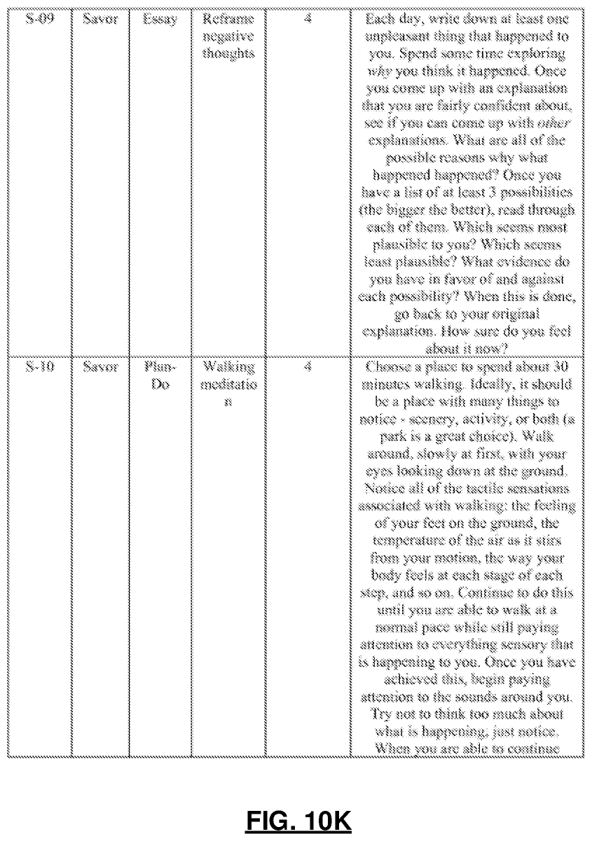

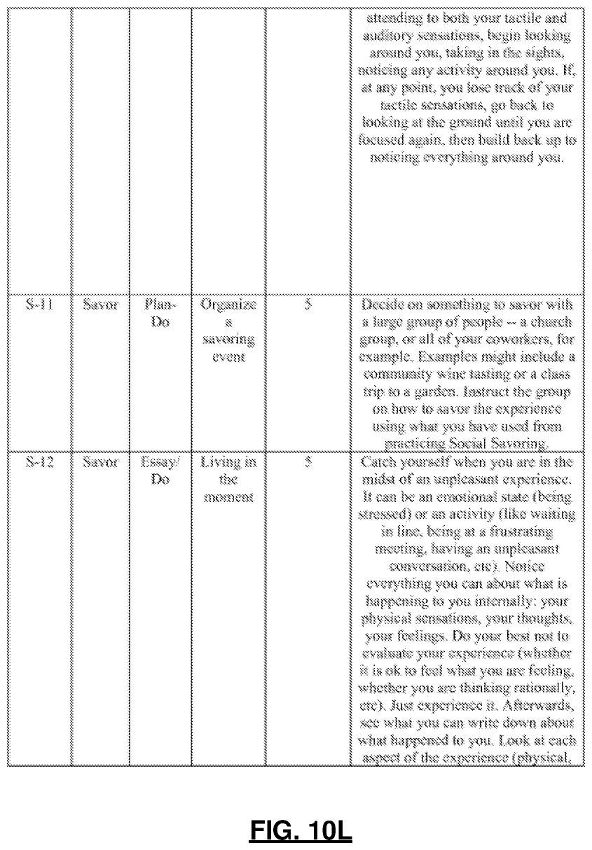

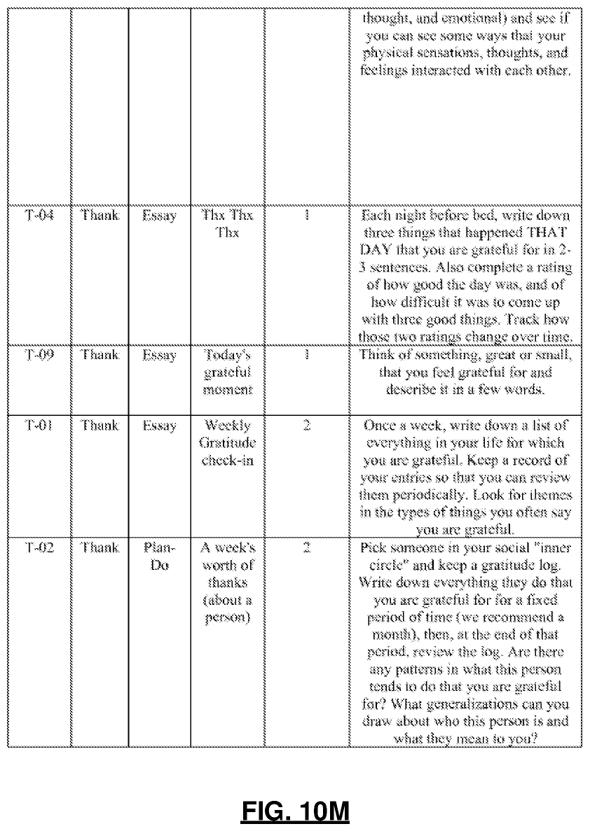

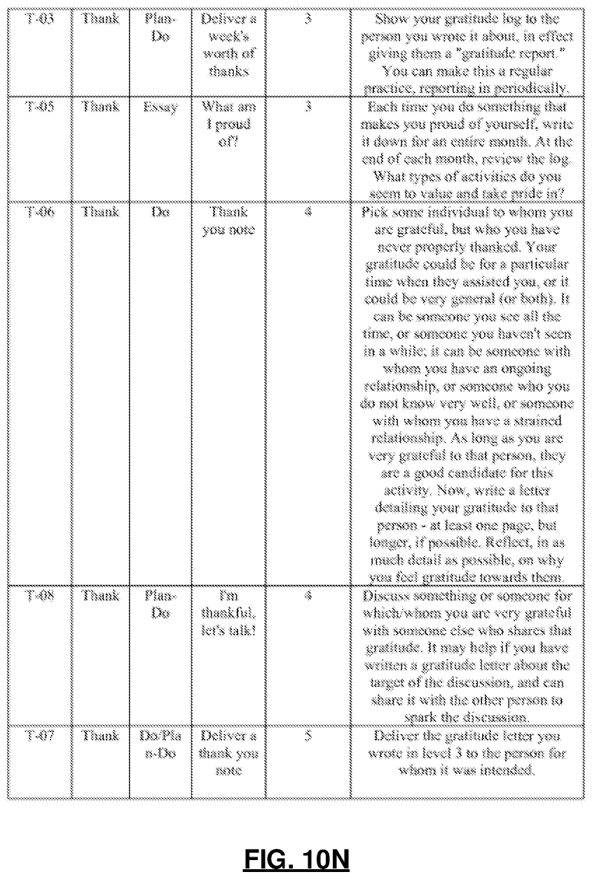

The STAGE framework of the app captures the essence of the science of positive psychology and allows for presentation in an accessible way. The STAGE framework of the app offers different types of science-based activities to users. The app provides nearly sixty science-based activities in various tracks to help users build the following five essential happiness skills: (1) Savor—Noticing the goodness around you and taking time to prolong and intensify your enjoyment of the moment. Savoring can involve the past (reminiscing), the present (mindfulness), or the future (positive anticipation); (2) Thank—Practicing gratitude; identifying and appreciating the things we have and the people in our lives; (3) Aspire—Feeling hopeful, having a sense of purpose and meaning in our lives, being optimistic; (4) Give—Performing acts of kindness; being generous and forgiving; and (5) Empathize—Imagining and understanding the emotions, behaviors, or ideas of others; having compassion. See FIGS. 10A-10N for details. Other and modified details regarding tracks are presented hereinbelow.

The framework of the app may provide multiple suggested tasks for each activity. For example, once the “reporter” activities are determined for each track part, the app provides 2-3 suggested tasks for each activity. These tasks retain the essence and the science of the proven intervention activity, but make sense within the theme of the track. The tasks are fun, and yet give clear and concise directions. A user needs to pick one of these tasks to complete in order to get credit for the activity. That is, users may only need to complete one of the task options in order to get credit for a given activity. When a user selects an activity, s/he can choose one of the two suggested tasks or a third “You Decide How” (YDH) option. Each suggested task is accompanied by a “Why It Works” section, which includes science references and explains why the activity is useful and how it relates to happiness. Below are some examples of sample activities and suggested tasks. Comprehensive lists of tracks and activities are provided both in a table shown in FIGS. 10A-10N as well as hereinbelow. An example of a track and its activities and tasks is shown in a table in FIGS. 11A-11C.

For example, for the track Feel More Loved by My Partner, and activity Today's Grateful Moment [Skill: Thank], a Suggested Task #1 may include the following. Name: The Little Stuff Counts, e.g., think of the reason you first fell in love with your partner or spouse—a trait or characteristic he/she still holds today. It could be his sense of humor, her kind generosity, or maybe his sex appeal. Write down some thoughts and spend a minute appreciating those same traits today. A Suggested Task #2 may include the following. Name: Thanks, Partner!, e.g., think of one good thing that happened today involving your partner or spouse. Write it down and add a few details about how it made you feel and the role you played, if any, in the positive experience. A You Decide How (YDH) task may include the following. For example, think of something, great or small, that you feel grateful for and describe it in a few words. Add a photo too if desired.

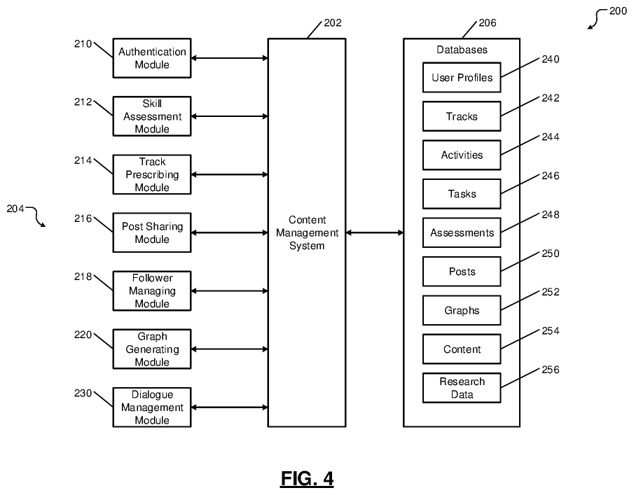

FIG. 4 shows a block diagram of the app described above, which is shown as the app 200. The app 200 comprises a content management system (CMS) 202, a plurality of modules 204 controlling various features and aspects of the app 200 described above, and a plurality of databases 206 associated with and utilized by the respective the plurality of modules 204 and the CMS 202. The CMS 202 manages the overall content provided by the app 200 to the users of the app 200 using the plurality of modules 204 and the plurality of databases 206.

The plurality of modules 204 comprises an authentication module 210, a skill assessment module 212, a track prescribing module 214, a post sharing module 216, a follower managing module 218, a graph generating module 220, and a dialogue management module 230. The authentication module 210 establishes user accounts and controls the users' access to the app 200. The skill assessment module 212 assesses a user's skills initially when the user signs up and later periodically as the user performs the prescribed activities. The track prescribing module 214 prescribes the tracks and modifies the tracks to the users according to their skill assessments as described above. The post sharing module 216 manages publication of the posts shared by the users, e.g., keeping them private or publishing them depending on the users' preferences, handling the likes and comments on the posts by other users, etc.). The follower managing module 218 manages the follower recommendations to the users based on profile matching as described above. The graph generating module 220 generates the happiness graphs as described above. The dialogue management module 230 conducts dialogues between the users and the app 200 and includes the dialogue management system as described below in detail.

The plurality of databases 206 comprises a database for each of user profiles 240, tracks 242, activities 244, tasks 246, assessments 248, posts 250, graphs 252, content 254, and research data 256. The app 200 provides content to the users of the app 200 using the plurality of modules 204 and the plurality of databases 206 under the control of the CMS 202.

The invention of the present disclosure may be an apparatus configured to increase the efficacy of a computer-implemented migraine treatment plan and adherence to said treatment plan. The apparatus may comprise at least one processor, at least one display, at least one memory comprising computer-executable instructions. The instructions may cause the apparatus to generate an interactive session comprising a plurality of tracks, each of the plurality of tracks comprising one or more activities. The aforementioned interactive sessions, tracks, and activities may be any embodiment of session, track, or activity described herein.

In an embodiment, the apparatus may store a baseline user metric (for example, a baseline user happiness level) and a variable user metric (for example, a variable user happiness level). The baseline user metric and the variable user metric may each correlate to one or more characteristics of a patient. As a non-limiting example, each of the happiness levels may be quantifications of the patient's contentment. The variable user metric may be updateable as a function of progress through the plurality of tracks. Upon signing up for the “app,” the patient may undergo an assessment session configured to determine the baseline user metric. The assessment session may be any of the tracks, activities, or tasks described herein, however, the results of the assessment sessions may be arranged to solely impact the baseline user metric, and not other user metrics or characteristics. Thus, the assessment may be configured to receive an initial set of user information. For example, the initial set of user information may be received during or after the generation of the assessment sessions. The set of user information may be text, audio, video, or another medium received by the apparatus in response to the session prompts. Therefore, the baseline user metric may be determined according to the assessment and the initial set of user information. In a further aspect, the apparatus may set a need set. The need set may be determined based on at least the baseline user metric and the variable user metric. That variable user metric may be a measurement tracking the patient's quantified happiness throughout their progress in the “app.” The need set may include a selection of one or more of the plurality of tracks, wherein the selection of one or more of the plurality of tracks is configured to increase the variable user metric. In effect, the need set may cause the apparatus to display the tracks which have been determined to increase the patient's variable user metric. Moreover, an increased variable user metric may be correlated to an increased treatment efficacy and effectiveness.

In a further aspect, the plurality of tracks comprise an interactive dialogue module, wherein, when engaged in the interactive dialogue module, the apparatus is configured to receive a user input. The interactive dialogue module is described in further detail below. The interactive dialogue module may comprise a three-tier architecture comprising a master file, a plurality of skeleton files, and a plurality of skin sets, wherein each of the plurality of skin sets is nested within one of the plurality of skeleton files and the plurality of skeleton files are nested within the master file. Each of the master file, skeleton files, and skin files, may be libraries of text, conversational elements, templates, and other dialogue components. Each of the plurality of activities may include one or more tasks, wherein the plurality of tracks correspond to the master file, wherein the plurality of activities correspond to the plurality of skeleton files, and wherein the one or more tasks correspond to plurality of skin sets. For example, if the apparatus is displaying task A of activity A of track A, the apparatus may generate dialogue from the skins that correspond to task A, the skeleton files that correspond to activity A, and the master dialogue file that corresponds to track A. Accordingly, the apparatus may be configured to identify a selected skeleton file and a selected skin set according to an instant task, wherein the instant task is the one of the one or more tasks engaged on the apparatus. As a result, the apparatus may generate and display a message according to at least the selected skeleton file.

In an embodiment, each of the one or more tasks and each of the plurality of activities comprise one of a plurality of difficulty levels, wherein each of the one or more tasks and each of the plurality of activities are unlocked according to a corresponding difficulty level relative to the variable user metric. In effect, the session may generate progressively more difficult tracks, activities, and tasks, to increase the efficacy of the treatment being delivered to the patient. In such an embodiment, the apparatus may also associate one of a plurality of badges to a user profile, wherein each of the plurality of badges correspond to completion of a given task, activity, or track. Thus, the patient's progress may be tracked by distributed tokens. The ‘reward’ aspect of the badges may instill positive feedback in the individual, increasing efficacy of the apparatus.

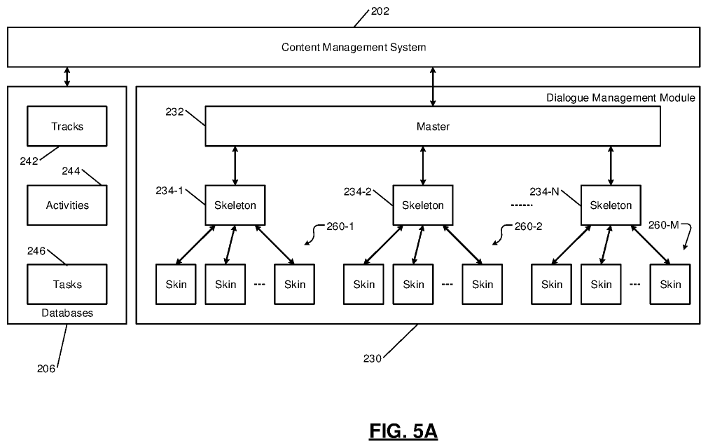

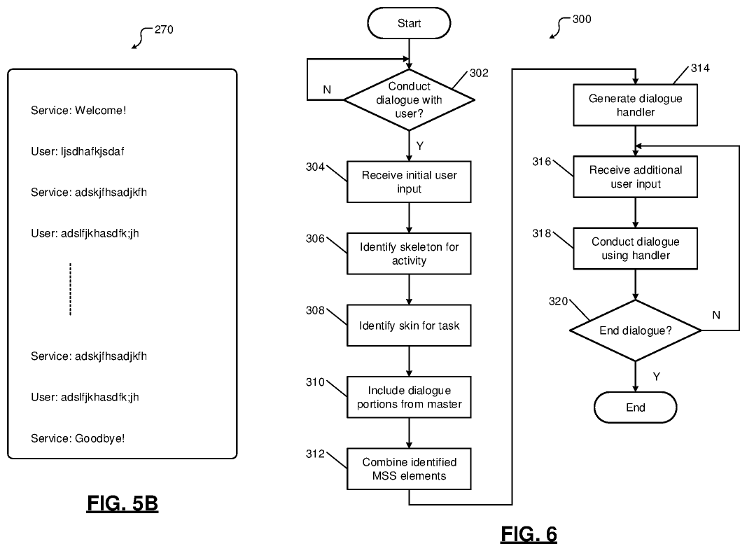

FIGS. 5A and 5B show the dialogue management system 230 in further detail. FIG. 5A shows the dialogue management system 230 having a 3-tier or 3-layer architecture. FIG. 5B shows an example of a dialogue box (or a dialog box) 270 on a user's device, e.g., a client device 120-1 shown in FIGS. 1 and 2). Throughout the present disclosure, the various “dialogue files” can also be called the respective “dialog files.”

In FIG. 5A, the dialogue management system 230 includes a single master dialogue file (also called a master file or a master) 232, and a plurality of skeleton dialogue files 234-1, 234-2 to 232-N, where N is the number of activities 244. N may be 60, 100 or even higher. These may collectively be called skeleton dialogue files 234, skeleton files 234, or skeletons 234. For each of the skeleton dialogue files 234, the dialogue management system 230 includes a plurality of skin dialogue files 260-1, 260-2 to 260-M. These may collectively be called skin dialogue files 260, skin files 260, or skins 260. The skin dialogue files 260 may include You Decide How (YDH) skin files and task skin files. Throughout the present disclosure, an individual skin file (YDH or task), a YDH skin file, and a task skin file are also referenced by the numeral 260. The dialogue management system 230 and its components, which include the master dialogue file 232, the skeleton dialogue files 234, and the skin dialogue files 260 are described below in further detail.

The dialogue management system 230 allows users to engage in a dialogue with the app 200 about an experience related to performing a prescribed activity 244. Dialogue boxes are generated using a tiered system of files, each with a unique purpose. An example of a dialogue box shown in FIG. 5B. Specifically, the dialogue boxes may be created using three sets of tiered or layered files: a single master dialogue file (master) 232, a plurality of skeleton dialogue files (skeletons) 234, and a plurality of skin dialogue files (skins) 260. The dialogue management system 230 that creates the dialogue boxes may include three layers of files—master, skeleton, and skin (MSS)—and can also be called a MSS system. Note that theoretically there can be multiple master files 232; however, practically, having a single master file 232 simplifies the design of the dialogue management system 230. Alternatively, the master file 232 may be eliminated from some or all dialogues.

While a track 242 may include many activities 244, the dialogue management system 230 includes a hierarchical architecture that leverages some amount of overlap that exists across the activities 244. The dialogue management system 230 may include a single master file 232 for all the activities 244, one skeleton file per activity 244, and one skin file 260 per task 246. The master dialogue file 232 may include the entire and complete markup language or script based structure that to run any dialogue, i.e., for each activity 244 and task 246.

The master dialogue file 232 may, they need not, be a JavaScript Object Notation (JSON) file or an Extensible Markup Language file. The dialogue management system 230 may have a master dialogue file 232 that represents a set of capabilities of the dialogue management system 230. The texts in the prompts, buttons, choices, and responses in the master dialogue file 232 may be fairly generic. For example, in the master dialogue file 232 a response after a user makes a single choice might be “Response to first choice.” This allows the master dialogue file 232 and its HTML based structure to work in any context for any activity 244.

A skeleton dialogue file 234 represents the specific structure for an activity 244, e.g., a skeleton can be designed for S-01 Savor the Small Stuff). The skeleton dialogue file 234 is a JSON file that makes selected references to the HTML structure in the master dialogue file 232 through the use of “include” statements.

A skin file 260, i.e., one of the skin files 260 corresponding to the skeleton file 234 associated with the activity 244) represents actual text to be presented when running a skeleton dialogue file 234 as well as the specific names for variables called life graph variables (LGVs) to be saved for a skeleton dialogue file 234. A skin file 260 is a spreadsheet, a comma separated value (CSV) file or similar data file that specifies the location of strings of text and the text to be used in a dialogue.

The dialogue management system 230 includes two layers of skins 260. Every skeleton dialogue file 234 has an associated overview or You Decide How (YDH) skin file 260. Additionally, a task skin file 260 can also be assigned to a specific task 246, e.g., there would be a specific task skin 260 for S-01-T-27 Smell the Roses).

Running a dialogue may involve identifying a skeleton dialogue file 234 (for example, the skeleton for S-01 Savor the Small Stuff) and a skin file 260 (for example, the skin for S-01-T-27 Smell the Roses). The activity base skin can contain instructions for how to further customize. “Compiling” the dialog uses the master and skeleton assets. Once a dialog is compiled, it is no longer dependent. That is, the master and skeleton and skin could be deleted and the hpml dialog would run just fine. This is true in that the MSS artifacts are used to produce the runtime artifact.

One way to initiate a dialogue involves the master 232, the skeleton 234, and the skin 260 being combined or compiled offline in the CMS 202. A potential optimization would do this in runtime on demand at the time of invocation of the dialogue. The advantage of the former way is that the availability of a full development environment allows the CMS 202 to manage different versions of each master 232, the skeleton 234, and the skin 260 and identify and debug errors if compilation fails.

More specifically, the master dialogue file 232 is sometimes a single file. For example, only one version of the master dialogue file 232 may exist on the server, i.e., in the app 200) at a given time. The master dialogue file 232 can be edited and updated over time, e.g., via the CMS 202), but in ways that overwrite the prior version. The master dialogue file 232 includes all of the core logic needed to determine and lay out the flow of any dialogue that can occur on the dialogue management system 230. The master dialogue file 232, therefore, is comprehensive and non-specific.

For example, the master dialogue file 232 may include the code necessary to run any language modeling and analysis algorithms, performing tasks such as the natural language classifiers (NLCs), Named Entity Recognition, Sentiment Analysis, and Linguistic Style Analysis and Transformation. For example, such algorithms include but are not limited to machine learning, deep learning, neural networks, statistical pattern recognition, semantic analysis, linguistic analysis, and generative models. A final user-facing dialogue may rely on the analysis of user input, e.g., one or two NLCs).

Every potential choice point that can occur in the flow of a dialogue may be coded into the master dialogue file 232. The master dialogue file 232 may include placeholder text that is very broad and generic, e.g., “Response to user”; or e.g., choices for the user can be “Choice 1” and “Choice 2”). Alternatively, the default text, where breadth is not required, can be specific, such as ending the dialogue with “Goodbye” or offering the user choices such as “Yes” and “No”.

Skeletons 234 and skins 260, i.e., the skeleton dialogue files 234 and the skin dialogue files 260, are where specific conversations and interactions with the user are often designed. The dialogue management system 230 may include a skeleton dialogue file 234 for each core activity 244 offered to the users, e.g., the app 200 includes nearly 60 activities. A skeleton dialogue file 234 may be a decisive, singular manifestation of the conversation flow offered by the master dialogue file 232. For example, if the objective is to interview the user about a relationship with a person in the user's life and the user's favorite things about that person, the skeleton dialogue file 234 for this interview can clearly delineate the flow for this conversation. The flow in the skeleton dialogue file 234 is deterministic, such that a series of given inputs from the user create a specific, exact conversation with the dialogue management system 230. However, the flow in the skeleton dialogue file 234 is dynamic, and a different set of user inputs can create a different conversation with the dialogue management system 230.

A skeleton dialogue file 234 may utilize only a small portion, e.g., 20% or 10%, of the dialogue portions or sub-dialogues defined in the master dialogue file 232. A skeleton dialogue file 234 may also use the dialogue portions of the master dialogue file 232 more than once. No specific text is determined by the skeleton dialogue file 234. So the skeleton dialogue file 234 can carry over the default text defined by the master dialogue file 232.

Furthermore, there can be an overlap between some of the activities 244. In such instances, the skeleton dialogue files 234 for such overlapping activities 244 can utilize the same or similar dialogue portions of the master dialogue file 232. Further, these dialogue portions in the master dialogue file 232 themselves can be reduced in number based on the overlap in some of the activities 244, which results in optimization in the design of the master dialogue file 232 and which provides additional synergy between the skeleton dialogue files 234 and the master dialogue file 232.

A skin dialogue file 260, i.e., each one of the skin dialogue files 260 includes a list of “specifics” which describes the exact sentences and phrases to be used by the dialogue management system 230 at each point in the conversation flow described by a given skeleton dialogue file 234. Skin dialogue files 260, therefore, are inherently tied to a specific skeleton 234 and are not paired with other skeletons 234. The dialogue management system 230 includes a skin dialogue file 260 for each specific task 246 for an activity 244 offered to users by the app 200. For example, for the nearly 60 core activities, the dialogue management system 230 includes anywhere from dozens to hundreds of skin dialogue files 260 for each activity 244.

In some cases, the default text in the master dialogue file 232 can suffice, such as giving the user a choice between “Yes” and “No”. In these cases, the skin dialogue file 260 can include an indication such as a null entry, allowing the text to be determined by the master dialogue file 232. If the master dialogue file 232 is subsequently changed so that these choices respectively become “Absolutely” and “No way,” these changes are automatically reflected in any conversation where the skin dialogue file 260 has null entries at these points. For the most part, however, the skin dialogue files 260 determine the response text, and the skin dialogue files 260 often overwrite the default responses of the master dialogue file 232.

Every skeleton dialogue file 234 has paired with it a You Decide How (YDH) skin dialogue file 260 that is designed in a broad, general way depending on the scope of the conversation determined by the skeleton dialogue file 234. For example, if a savoring skeleton dialogue file 234 is built to help the user savor a positive feeling, the YDH skin dialogue file 260 can determine all the sentences and phrases for this conversation. However, a new skin dialogue file 260 may be created from a base skin that focuses the user specifically on savoring food. A different skin dialogue file 260 may be created from this YDH skin 260 that focuses the user specifically on savoring an experience. Notably, due to the tiered architecture of the dialogue management system 230, no changes are required at the master 232 or skeleton 234 level to add this new activity. The only edits needed are to the YDH skin dialogue file 260, where any new phrases or guidance specific to food (or experience) can be added or edited. This new skin dialogue file 260 can then be paired with the savoring skeleton 234 to run a food (or experience) savoring conversation. Due to the tiered architecture of the dialogue management system 230, this versatility is accomplished without requiring code changes at the master 232 or skeleton 234 level. This significantly simplifies the design of the dialogue management system 230.

The master dialogue file 232 can offer a broadly-defined capability to identify an object of the conversation. The master dialogue file 232 includes the built-in architecture (CHTML based data structures) to receive variables that can decide how the object is identified, how many questions are asked of the user, whether or not to provide a response at certain points, etc. The skeleton dialogue file 234 is where the flow-determining variables that are fed to the master dialogue file 232 are defined. Accordingly, the result of designing a skeleton dialogue file 234 is the decision to use the identify capability to ask two questions, for example, and respond any time the user identifies an emotion or an activity 244 based experience. The skin dialogue file 260 paired with the skeleton dialogue file 234 defines, among all of the dialogue's specific text, the questions that can be asked, which for one particular skin dialogue file 260 may be “What is your favorite hobby?” and “How do you feel when you are engaging with this hobby?”. The skin dialogue file 260 paired with the skeleton dialogue file 234 additionally defines the full set of potential responses to emotions that might be provided in the answer by the user.

The master dialogue file 232 includes a library of sections or dialogue portions, each of which is a subset (or sub-dialogue) of a conversation that is focused on a single task 246 and includes distinct pieces of a conversation designed to achieve a goal in the conversation. Only a few of the dialogue portions are used during a dialogue. Further, some of the same dialogue portions may be used in combination with other dialogue portions in another dialogue. Essentially, for conducting a dialogue about an activity 244, a few of the dialogue portions from the master dialogue file 232, a skeleton dialogue file 234 corresponding to the activity 244, and a plurality of skin dialogue files 260 corresponding to the tasks 246 associated with the activity 244 are compiled together.

The dialogue management system 230 conducts the dialogue with the user in a versatile, life-like manner using the compiled combination of the dialogue portions from the master dialogue file 232, the skeleton dialogue file 234, and the skin dialogue files 260. This method of conducting dialogues eliminates the need to have a one to one correspondence between the number of dialogue portions of the master dialogue file 232 and the number of activities 244. For example, the dialogue management system 230 may include only a few sections, 10-20, about 60 activities and a much greater number of tasks 260. Accordingly, this method, comprising generic, modular, and reusable data structures designed in the master file 232, which are then selected by the skeleton 234 and modified by the skins 260, results in significant improvements and optimizations in the architecture and resource utilization of the databases of the app 200.

In a conversation, i.e., in a dialogue, a node is an atomic element. A node typically includes a prompt for the user and includes logic to process the user's response to the prompt. The prompt and the user's response (user input) can include one or more of text, speech/audio, and video including virtual reality (which can be used to extract body posture/positions facial expressions etc. for use as user input). Based on the processing of the response, the conversation moves to a next node. A section or dialogue portion in the master file 232 includes a group of nodes.

There are two primary types of sections in the master file 232: linear (or sequential) sections and adherence sections. The nodes in the sequential sections may be processed sequentially, i.e., a next node is processed when a condition is satisfied after processing a prior node. In an adherence section, after a node is processed, control always returns to the first node, and a check is performed as to which, if any, variable remains to be filled, and control moves to that node for which a variable needs a response. The process is repeated until all the variable are filled or until a counter expires. In case of a non-ending loop, e.g., due to repeated irrelevant responses from the user, a counter is maintained, and the loop is exited on expiration of the counter. The counter is only an example; instead, any other stopping condition that is guaranteed to be met within a reasonable number of conversation turns can be used.

Across the different sections or dialogue portions of the master 232, while the prompts may be different, and the content of the text (in the user response) may be different, the structure of the sections may be kept fairly steady across different activities 244. For example, in a conversation, regardless of the activity 244, the dialogue may start with a greeting and may end with a summary, both of which can be short, repeatable, i.e., reusable sequential sections. The dialogue may additionally include an adherence section to elicit responses for a few variables needed to conduct the dialogue. The dialogue may further include another section to clarify or disambiguate an item, for example.

These sections tend to have similar structures though different content. Further, irrespective of the number of activities 244 offered by the app 200, these sections of the master file 232 are few in number, i.e., they are not as many in number as the number of activities 244; or there is no one to one correspondence between the sections of the master file 232 and the activities 244. Accordingly, the master file 232 includes only a handful of sections and is a collection or an array of a few sections that (can but) do not include any specific content, e.g., what to ask, but have variables with generic values that can be and are usually overwritten by the skeleton 234 and the skins 260.

The skeleton file 234 simply contains a series of include calls that select a few sections (dialogue portions) from the master file 232 to accomplish the dialogue at hand. At this point, however, the dialogue management system 230 does not know the exact nature of the dialogue, e.g., whether the user wants to savor an experience or food. The skeleton 234 therefore also includes an identify section from the master file 232, which is very generic in nature, e.g., it can identify a person, an object, etc.

The values for the variables in these sections are provided by the skin file 260. These values are elicited from the user by the skin 260 by prompting the user with questions, e.g., multiple choice questions. The YDH skin file 260 is also general in nature, e.g., it can indicate savoring something but cannot further specify an experience or food. The task skin 260 provides the specific values for the variables that override the generic values of variables as well as specific values provided by the master file 232, if any. These features of the master file 232, the skeleton files 234, and the skin files 260 eliminate the need for providing custom dialogue scripts by anticipating every input from users, which again greatly simplifies the design of the dialogue management system 230.

The specific features or data structures employed by the master 232, the skeletons 234, and the skins 260 are now described. Throughout the remainder of the disclosure, while references are made to natural language classifiers (NLCs) and associated variables and values, NLC is used only as an illustrative and non-limiting example of a task performed by language modeling and analysis algorithms mentioned above.

The master dialogue file 232 includes the following features or data structures that are implemented in markup language or scripting language: conditional values, default NLC values, and a single array. In the conditional values features or data structures, as part of a variable/value pair, a capability to assign values based on a condition is provided, e.g., response_text can be assigned to a string based on the value_of emotion. For the first condition that evaluates as true, the variable assignment is made, and no further conditions are evaluated. Unless defined, by default the “else” condition is equal to the current value of the variable, e.g., in the above example, the “else” value can be “_response_text”.

In the default NLC values features or data structures, as part of the initial attributes of a section within the Script, included is an attribute named “nlc_defaults” which specifies what the output of a classifier should be depending on whether a classifier is used or not. Each classifier used in a section (dialogue portion) is identified by name and a default value is defined. If a classifier is present in a section (dialogue portion) and a default is not defined under nlc_default, the default value is a blank string.

In the single array of variables feature or data structure, for each choice within a single (or multi) input request, three attributes are defined: a “label”, an “Lgv_value”, and a “prompt”, with each choice identified by a “name” to the left of the colon, and the three attributes as strings defined to the right of the colon. The first attribute, “label”, is the text that should be presented as a choice to the user. The following two attributes are accessible as attributes of sensor objects after a selection is made. Accordingly, an Lgv_value(sensor) is an Lgv_value text of a choice that is made, and a prompt(sensor) is a prompt text of the choice that is made. In other words, to illustrate, if a user choses a third option, for example, Lgv_value(sensor)==‘third choice text’ and prompt(sensor)==‘Response to third choice’. If the “label” of a choice is blank, then that choice is not presented. If every choice has a blank label, a validation error should occur (however, this happens at the level of the skeleton 234 and skin 260; the master 232 allows for all blank values that should be filled in at the skeleton/skin level).

The skeleton dialogue file 234 may contain “include” calls for selected dialogue portions from the master dialogue file 232, including both variable folders, global handlers, and sections (dialogue portions). The following feature or data structure may be implemented for the skeletons: NLC Switches, Variable Assignments, and Section-to-Section Flow. In the NLC switches features or data structures, as an attribute of an included section (dialogue portion) in the master 232, “nlc_active” defines whether a classifier is run or not in that section (dialogue portion). The “nlc_active” attribute defined in the skeleton works in conjunction with the “nlc_default” attribute defined in the master dialogue file 232. When “nlc_active” for a classifier is set to false, the output of the classifier is the default defined in “nlc_default”. By default, each classifier present in an included section (dialogue portion) has an “nlc_active” value of false. So unless the skeleton dialogue file 234 defines an NLC as active (set to true), that classifier will not run in this section (dialogue portion).

In the variable assignments features or data structures, as an attribute of an included section (dialogue portion), “assign” redefines values for certain variables found in that section (dialogue portion). For any variable present in the section (dialogue portion) and not included in the “assign” list, the value remains as it is defined by the master dialogue file 232. However, the “assign” values made by the skeleton dialogue file 234 override the values set by the master dialogue file 232. Functionally, the assign values help define the flow and structure of an included section (dialogue portion), allowing importing a single block of code that can be used differently depending on the value of these variables. This feature is not merely better code but rather a better data structure architecture that yields efficiencies in database design and resource usage and significantly improves the functioning of the databases as one skilled in the art can appreciate.

The section-to-section flow feature or data structure is as follows. The master dialogue file 232 has “next”/“goto” statements that reference every section, i.e., dialogue portion within the master dialogue file 232. When a skeleton dialogue file 234 includes only a subset of the sections (dialogue portions) from the master dialogue file 232, references to those sections (dialogue portions) that are not included in the skeleton dialogue file 234 need to be handled. The master dialogue file 232 includes three “identify” sections (dialogue portions) named “identify”, “2nd_identify”, and “3rd_identify”. For example, a given skeleton dialogue file 234 may include only the “identify” and “2nd_identify” sections (dialogue portions). In the “2nd_identify” section (dialogue portion), the master dialogue file 232 has “next”/“goto” statements pointing to “3rd_identify”, which does not exist in this skeleton dialogue file 234 in this example. At runtime, this skeleton dialogue file 234 should simply move to the identified section (dialogue portion) in the master dialogue file 232 (the “3rd_identify” section or dialogue portion in this example) and then look sequentially section by section for the next section or dialogue portion that the skeleton dialogue file 234 actually does include.

In the skin dialogue files 260, there may be two levels of skins. A YDH (or overview) skin, and a task skin. The skin dialogue file 260 can be in a spreadsheet format but can ultimately run as a comma separated value (CSV) file in the content management system (CMS) 202 of the app 200. First few top rows under the headers rename the life graph variables (LGVs) used by the skeleton dialogue file 234. For every instance of the LGV name in the “Original” column, it is replaced with the name in the “Value” column across the entire skeleton dialogue file 234. If an LGV in the skeleton dialogue file 234 is either not referenced here or has a blank value in the “Value” column, the original name persists. Subsequent rows redefine the text of the skeleton dialogue file 234. The text in the “Original” column is a reference to the text in the master dialogue file 232 at that location. The “Value” column is the new text that replaces the existing text from the master dialogue file 232. If the “Value” column is blank, the value from the master dialogue file 232 persists. But the priority is given to the skin 260. Ideally, the YDH skin 260 can be automatically generated from a skeleton dialogue file 234 in the CMS 202 by identifying every LGV and every segment of text. An exported skin created by the CMS 202 would have an empty “Value” column. An “Author” column designates whether or not this row is to be included in an automatically generated task skin 260. A “0” indicates it is not included, and a “1” indicates that it is included.

The task skin 260 can be automatically generated from the YDH skin 260 by: (1) removing the rows with “Author” designated as “0” and then removing the “Author” column altogether; (2) assigning each “Value” entry of the task skin 260 as the “Value” entry of the YDH skin 260 if it's not empty or the “Original” entry of the YDH skin 260 if the “Value” entry is empty; (3) creating an empty “Value” column; and (4) adding a “Legacy” column with one cell automatically populated with the “Short text”, “Description text”, and “Short text labels” already in the CMS 202 for the designated task 246. For each of these legacy task attributes, a tag is present that defines and separates the different strings. The “Value” column can then be filled in. When the CMS 202 is running an activity 244 using a task skin 260, it first prioritizes the “Value” entries from the task skin 260; if those are empty, next prioritizes the “Value” entries from the YDH skin 260; and if those are also empty, lastly prioritizes the “Original” entries from the YDH skin 260. If all of these values are blank for an “ask”/“prompt” or “next”/“text” entry, the dialogue management system 230 does not create a text bubble and continues with the flow of the dialogue. As described above, if the value for a single/multi label is blank, then it is not shown, and if all the labels for a single/multi input are blank, there is a validation error. The task skin file 260 is still paired with the original skeleton dialogue file 234. Accordingly, for example, to run S-01 Savor the Small Stuff in “You Decide How” mode, the dialogue management system 230 pairs the S-01 skeleton dialogue file 234 with the S-01 YDH skin file 260; to run S-01-T-27 Smell the Roses, the dialogue management system 230 pairs the S-01 skeleton dialogue file 234 with the S-01-T-27 task skin file 260; and so on.

In FIG. 5B, the user initiates the dialogue 270, e.g., using a drop down menu from the app 200, which is presented on the user's device, e.g., client device 120-1 in the form a user interface (UI). For example, the dialogue box 270 can appear similar to the UI of a text messaging app on a smartphone. In the dialogue 270, the entity “Service” represents an automated conversational agent driven by the 3-tier architecture of the dialogue management system 230 described above.

The dialogue 270 can begin with a greeting. The dialogue 270 can end with a summary and/or another greeting. The dialogue 270 provides the app 200 (via the dialogue management system 230) another opportunity, in addition to the tracks 242, activities 244, and tasks 246, to effect an intervention, for example, by coaching the user on a particular happiness skill such as how to practice empathy or how to improve practicing empathy. The dialogue 270 also offers the user the opportunity to share his or her experience, exhibit his or her skill level regarding a particular happiness skill via the dialogue 270, and improve the particular happiness skill based on the coaching received from the app 200 via the dialogue 270.

While not shown, the dialogue 270 can include text message as well as audio/video messages from either or both of the service and the user. Further, the dialogue can also include graphics such as emoticons, photos, videos, music, and so on that can be exchanged by and between the service and the user, i.e., either or both of the service and the user can also provide the graphics such as emoticons, photos, videos, music, and so on.

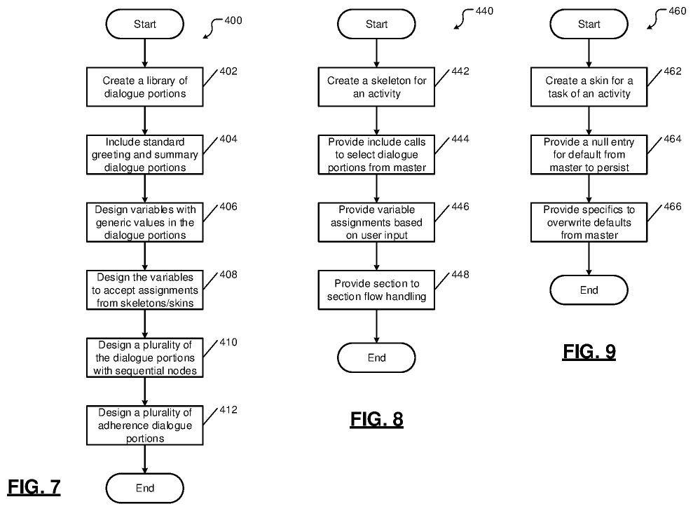

FIG. 6 shows a method 300 for conducting a dialogue between the app 200 and a user of the app 200 using the dialogue management system 230. For example, the method 300 is performed on one of the servers 130 and includes presenting the dialogue 270 on a user device such as the client device 120-1 via the distributed communications system 110.

At 302, the method 300 checks whether a user is initiating a dialogue 270 with the app 200. At 304, if a user initiates a dialogue 270 with the app 200, the method 300 receives an initial input from the user. At 306, based on the user input, the method 300 determines an activity 244 that the user wants to discuss in the dialogue 270 and identifies a skeleton file 234 for the activity 244. At 308, the method 300 identifies a skin file 260 for a task 246 associated with the activity 244. At 310, the method 300 includes dialogue portions from the master file 232 selected based on the activity 244 to conduct the dialogue 270. At 312, the method 300 combines the selected dialogue portions of the master file 232, the skeleton file 234 for the activity 244, and the skin file(s) 260 for the task 246, e.g., the method 300 compiles these master 232, skeleton 234, and skin 260 elements. At 314, the method 300 generates a dialogue handler generated based on the combination or compilation that is used to conduct the remainder of the dialogue 270.

At 316, the method 300 receives additional inputs from the user. At 318, the method 300 conducts the dialogue 270 with the user based on the user inputs using the dialogue handler, e.g., the method 300 interactively responds to the user inputs. At 320, the method 320 determines if the user wants to end the dialogue 270. The method returns to 316 if the user wants to continue the dialogue 270. Otherwise, the method 300 ends.

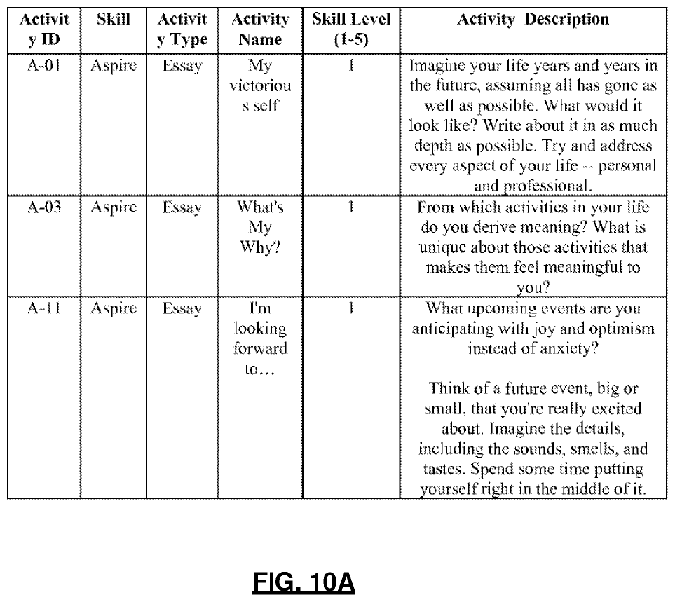

FIG. 7 shows a method 400 for designing and generating the master file 232. At 402, the method 400 creates a library of dialogue portions such that the number of dialogue portions is less than the number of activities 244, i.e., there is no one to one correspondence between the number of dialogue portions of the master file 232 and the number of activities 244 offered by the app 200. For example, the method 400 identifies and takes advantage of any overlap or redundancies across the activities 244 offered by the app 200.

At 404, in the library of dialogue portions, the method 400 creates a standard greeting dialogue portion to be presented at the beginning of any dialogue 270 irrespective of underlying activity 244, and a standard summary dialogue portion (or another standard greeting dialogue portion) to be presented at the conclusion of any dialogue 270 irrespective of underlying activity 244. At 406, the method 400 designs variables with generic values (and a few variables with specific values) in the dialogue portions of the master file 232. At 408, the method 400 designs or configures the generic variables to accept specific value assignment from skeletons 234 and skins 260. At 410, the method 400 designs a plurality of the dialogue portions of the master file 232 to include sequential nodes. At 412, the method 400 designs or configures a plurality of the dialogue portions of the master file 232 to function or operate as adherence dialogue portions.

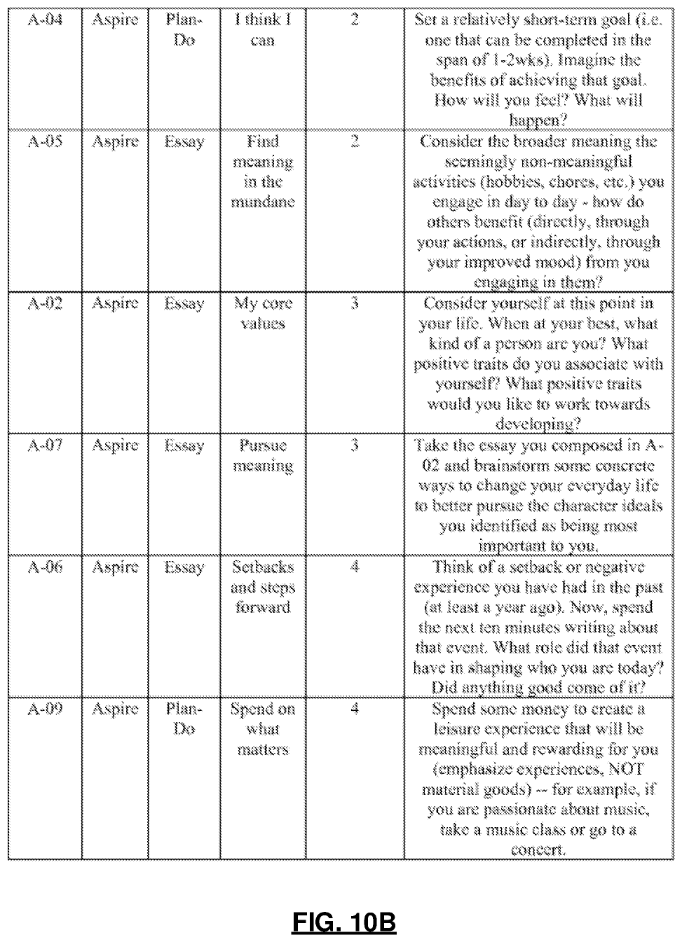

FIG. 8 shows a method 440 for designing and generating skeleton files 234. At 442, the method 440 creates a skeleton file 234 for an activity 244, i.e., the method 440 creates one skeleton file 234 per activity 244 offered by the app 200. At 444, the method 440 provides “include” calls in the skeleton file 234 to select relevant dialogue portions from the master file 232. At 446, the method 440 provides variable assignments to the selected dialogue portions based on user input to conduct the dialogue between the user and the app 200. At 448, the method 440 provides section to section flow handling to conduct the dialogue between the user and the app 200. For example, the order in which the flow of or between the sections is conducted during a dialogue may be different than the order in which the sections are arranged in the master file 232.

FIG. 9 shows a method 460 for creating a skin file 260. At 462, the method 460 creates a skin file 260 for a task 246 for an activity 244, i.e., the method 460 creates a skin file 260 for each task 246 of an activity 244 offered by the app 200. At 464, the method 460 provides an indicator such as a null entry to allow for a default value for a variable from the master file 232 to persist. At 466, the method 460 provides a specific value to overwrite a default value for a variable from the master file 232. The specific value is based on the user input and is passed to the skeleton file 234, which then assigns it to a suitable variable in a selected dialogue portion from the master file 232.

The dialogue management system 230 of the present disclosure differs from a chatbot. A chatbot is a very general description of any conversational agent that communicates with a user via text or voice/video on a turn by turn basis. A chatbot can therefore be intelligent, e.g., use machine learning or completely pre-scripted; so it is very broad in scope. The differences between the dialogue management system 230 of the present disclosure and a chatbot are in the specific applications and its 3-tier architecture based on the specific applications. The dialogue management system 230 does not focus on delivering efficacious psychological interventions in the best possible way, and on using machine learning and dialogue management mechanisms to accomplish that. Rather, the dialogue management system 230 is an efficient way to create and program a “chatbot” using the 3-tier architecture described above so that the scripts governing the dialogues do not have to be created for all possible conversational scenarios and so that the scripts governing the dialogues can reuse some code.

Further, the dialogue management system 230 of the present disclosure differs from other automated customer support systems. Specifically, the difference is due to the operation of the dialogue management system 230 based on the tracks 242, the activities 244, and the tasks 246, where the activities 244, about which dialogues are conducted, are recommended by the app 200. This schema of the app 200 creates a unique opportunity for designing the synergistic 3-tier architecture to conduct dialogues as described above. Unlike the app 200, systems that do not evaluate feedback from users regarding activities recommended by the systems and that do not attempt to improve user behavior via interventions offered based on the feedback, naturally lack the need for the 3-tier architecture described above. Of course, the dialogue management system 230 can be used with any other system that evaluates feedback from users regarding activities recommended by the system and that attempts to improve user behavior via interventions offered based on the feedback.

In sum, the dialogue management system 230 of the present disclosure uses a novel 3 layer approach—a generic master file 232 that can cater to dialogues on any of the nearly 60 activities offered by the app 200, a skeleton file 234 that is specific per activity 244 and that links to one or more “sections” or dialogue portions in the master file 232 (some of which can be reused for another activity 244), and a plurality of skin files 260 that handles the input and output at the user interface presented to the user as a dialogue box 270. For each dialogue 270, these 3 elements are combined and a dialogue 270 is conducted. For another user or another activity 244, another combination is used to conduct another dialogue 270. The synergy provided by the 3 tier approach is that the generic nature of the master file 232, the ability of the skeleton file 234 to include sections of the master file 232 in any combination as needed, and the ability of the skins 260 to provide the specific values to variables in the selected sections of the master file 232 result in significant reuse of the sections of the master file 232, which yields efficiencies in database design and use of database resources. The dialogue management system 230 is versatile in that it works across all activities 244 offered by the app 200 and regardless of the variations in the user's inputs and in the activities 244. Thus, the 3 tier design of the dialogue management system 230 improves the functionality of the computer databases 206, not merely code.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

In certain embodiments of the present invention, the app 200 embodies a service of various treatment and prevention disciplines, such as positive psychology, cognitive behavioral therapy, mindfulness, stress reduction, etc. One exemplary service is referred to herein for convenience as the “Program.” The Program is a novel, science-based app for engaging, learning and training the skills of happiness. The Program is based on a framework developed by psychologists and researchers in a collection of therapeutic disciplines such as Cognitive Behavioral Therapy, Mindfulness, positive psychology etc., and assists users in the development of certain skills related to being happy, for example, Savor, Thank, Aspire, Give and Empathize (or S.T.A.G.E.™). In certain embodiments, each skill is developed using various activities, ordered in increasing skill level, that gradually unlock as the user progresses in building that skill. With the Program, a user selects a “track” that contains sets of activities that are designed to address a specific life situation or goal.

As the user performs one or more of these activities, the Program system assesses and re-assesses the user's physical and emotional states using various tools. For instance, there may be a plurality of sensors, e.g., biometric that are placed within a vicinity of the user, e.g., in wired and/or wireless communication with the user's smartphone that extract biometric information from the user while the user is engaged in an activity or a task. Examples of such extracted biometric information are heart rates, heart rate variability, brainwaves, body heat, pupil dilations, etc. In another instance, one or more sensor mechanisms within the user's smartphone, e.g., speaker, camera, microphone, buttons, keys, etc. are used to capture user information. Examples of such captured information are recorded speech, typed texts, facial expression, etc. In a further instance, the user's physical or emotional states may be assessed from self-reports such as questionnaires. In other instances, a mix of foregoing information may be used concurrently to assess the user's physical or emotional states.

In accordance with the Program system, the extracted, captured and/or otherwise provided information are processed to analyze the user's feelings including, but not limited to, the user's reaction, the user's engagement level, the user's adherence level, the change in the user's psychological state, etc. in regards to the performed, or partially performed, the Program activities. Processing may be carried out within the Program application or by another processing unit that resides within the smartphone (or tablet or other computing system). Alternatively, the extracted and/or captured information are transmitted and processed remotely by a server (or other remote electronic device). In any of these versions, processing includes application of select mathematical algorithms and analytical computations on user input data obtained while the user performs the activities. The processing ultimately results in providing of select follow up activities that further enhance development of the happiness skill in order to achieve the desired outcome.

In further accordance with the Program system, the processing of data and/or the providing of follow-up activities is ongoing. In particular, as the user performs the provided activity, the Program system continually monitors and interacts with the user to obtain ongoing real-time information. For example, the ongoing real-time information may be a user's response to a question, what the user has done in response to a task, or various other biometric information of the user obtained from the sensor(s) placed within a vicinity of the user. With such real-time or aggregate analysis, the user's interaction with the Program system becomes more dynamic and results in higher levels of engagement as that interaction continues.

Further details of the Program system and operation of the Program system are set forth in U.S. patent application Ser. No. 14/284,229, entitled “SYSTEMS AND METHODS FOR PROVIDING ON-LINE SERVICES” and U.S. patent application Ser. No. 14/990,380, entitled “DYNAMIC INTERACTION SYSTEM AND METHOD,” and the entire contents of each of these applications is incorporated herein by reference. For the sake of brevity, further details of the Program system/service are not provided herein (except as otherwise described herein).

### Empathy

In accordance with the present invention, the computing system further dynamically responds to the user's actions and feedback by demonstrating simulated human emotion and/or human cognitive skill. In certain embodiments to be discussed, the computing system is configured to demonstrate empathy.

In further accordance with the present invention, a computing system is equipped or otherwise programmed with artificial intelligence for simulating a variety of human emotion and cognitive functions. For purposes herein, the term artificial intelligence (AI) means a machine or device suitably adapted or programmed in a manner sufficient so that the machine or device perceives its environment (or the desired environment) and takes actions that maximize its chance of successfully achieving its intended goals, as well as processes carried out by such machines or devices. The term AI can further mean the ability to learn from data and generalize unseen data by a machine. Display of artificial intelligence by a computing system generally includes performance of tasks that normally require a human intelligence. Various embodiments of the present invention are directed to demonstration of artificial “emotional” intelligence, which is a particular subset of human intelligence.

The field of artificial intelligence draws upon various diverse fields, such as computer science, mathematics, psychology, linguistics, philosophy and many others. In more recent years, AI has progressed to the point of understanding (at least from the machine's perspective) the aspect of human intelligence that is known as emotional intelligence, e.g., empathy. The term “empathy” generally is defined as the (human) ability to understand and share the feelings of another. In other words, empathy is the capacity to understand or feel what another person is experiencing from within the frame of reference of the other person. With recently developed AI emotion models, machines can now be programmed to learn when and how to display emotion in ways that enable the machine to appear empathetic or otherwise emotionally intelligent.

In accordance with the present invention, the above discussed Program system further interacts and engages with users in an empathetic and supportive manner to provide certain benefits as herein described. The system/process of the present invention, therefore, in certain embodiments, is capable of emotional intelligence and with such emotion intelligence, conveys empathy to users of the system to keep the user advantageously engaged over time.

### Mirroring Prompt

In certain embodiments, the inventive system includes artificial intelligence sufficient to provide the system with a so-called “mirroring” ability. As described herein, the inventive system in such certain embodiments employs various algorithms, such as topic analysis, natural language classification, etc. to reflect back on input received from the user and/or measurement data collected from the user, and then responds to the user with context-based responses.

In each of the embodiments described herein in which AI is employed by the inventive computing system to convey or simulate emotional intelligence, the environment presented to the user beneficially is human-like from the perspective of the user that results in a more rewarding or engaging environment to the user that, in turn, results in greater engagement by the user that, in turn, results in a far greater chance of success in the ultimate goal of achieving a greater level of happiness.

In accordance with the present invention, the “next” step in the interaction may depend on what rules have been set in regards to the provided activity. For example, the mirroring stage may be performed in a loop until the computing system decides to move onto the next question to ask. As another example, the next step may be based on the user's input. As a further example, the mirroring stage may be an interim stage that may be used at each “turn” of the interaction and the determination for the next turn may be based on adherence fidelity. Additional details of the adherence fidelity feature of the present invention is provided in the U.S. Provisional Application Ser. No. 62/533,423, filed on Jul. 17, 2017, the entire content of which is incorporated herein by reference.

The mechanism of mirroring entails maintaining the same flow of interaction with the user and including an appropriate “mirroring prompt” in the interaction. For example, when two people communicate, it has been scientifically researched that their brains tend to get activated in similar regions. This effect is also known as “brain mirroring.” See “Brain Basis of Human Social Interaction: From Concepts to Brain Imaging” by Hari, R., & Kujala, M. V., Physiological Reviews, 89(2), 453-479 (2009) for additional detail on brain mirroring, the content of which is incorporated herein by reference.

In accordance with an exemplary computing system of the present invention, the anatomy of a mirroring prompt can be outlined as follows: (1) Reflecting the content of what the user just said; (2) Using an understanding and supportive tone; (3) Using an emotional tone that is similar to the emotions the user conveyed or an emotional tone that is appropriate for the emotions the user conveyed; and (4) Addressing the context or situation that the user mentioned. The mirroring prompt demonstrates that the computing system “feels” what the user is feeling and, naturally, responds in a similar tone, mirrors the content of the conversation, conveys commiseration, etc.

Without mirroring, the computing system jumps to providing the user with solutions. However, with mirroring, the system has employed a mirroring prompt using a similar tone to reflect back “going back to school” and “feeling drained,” while mentioning that “everyone” feels drained from time to time, thus showing that it understands how the user is feeling. Again, similar to the first example, the user feels more appreciated and engaged with the conversation when mirroring is implemented.

As such, to better identify and understand the contents of the conversation, the computing system employs a set of techniques such as natural language classification, topic modeling, sentiment analysis, named entity extraction, emotion detection, etc. The list is not exhaustive and the computing system may employ additional techniques as necessary to identify and understand a broad spectrum of topics. The series of steps in applying various analytic techniques is also referred to herein as the computing system training a “classifier.”

Various details of topic or language modeling techniques that may be employed in certain embodiments of the present invention are not described, but rather are sufficiently and well understood in the art. Those details that are well known and understood are not described herein for brevity. Various publications that describe such techniques that may be employed herein include: “Probabilistic Topic Models” by Blei, D. M., Communications of the ACM, 55(4), 77-84, (2012); “Utopian: User-Driven Topic Modeling based on Interactive Nonnegative Matrix Factorization” by Choo, J., Lee, C., Reddy, C. K., & Park, H., IEEE Transactions on Visualization and Computer Graphics (Volume: 19, Issue: 12, December 2013); and “Hierarchical Topic Models and the Nested Chinese Restaurant Process” by Griffiths, T. L., Jordan, M. I., Tenenbaum, J. B., & Blei, D. M., Published in NIPS'03 Proceedings of the 16th International Conference on Neural Information Processing Systems, Pages 17-24 (Dec. 9-11, 2003), and each of these publications is incorporated fully herein by reference.

Next, the computing system runs additional clustering analyses to group together various themes and topics. For instance, this may require further grouping together themes and topics that may be facially different but nonetheless require a similar response to the user. For example, “working in the yard” and “being outdoors” may be grouped together as the mirroring prompt would be the same, e.g., “being outdoor is great!” regardless of whether the user is describing his or her effort in mowing the lawn or taking a leisurely walk in a park. Still further, this is particularly effective if the same response for different topics has the same psychological effect, as at the end of the day, the goal is to cater to the efficacy of a psychological intervention.

Once the reference data has been grouped into major themes via the steps described above, the computing system identifies the most representative text sample of the theme. The most representative text sample may be determined by scoring each text sample to assess its proximity or degree of match to each topic, and then using only the samples with the closest match (or top-scoring) as the most representative. Using these data, a text classifier is trained that can learn to distinguish between themes. For example, the text classifier can use features extracted from the text such as the topic scores or other language model scores, e.g., word2vec scores, and then use another classification algorithm, e.g., Bayesian classifier, support vector machine, deep learning, neural network, etc. to learn to distinguish between the features. In a case where voice or video data are used, the computing system may further include AN classification algorithms, such that the content beyond the text, such as the tone of the voice or the facial expression may also be used.

Some of the classification algorithms that are discussed above as being utilized by the Program system are also known in the art. Details of the specifics of the known algorithms are omitted herein for brevity. Instead, below list demonstrates exemplary publications that are incorporated herein by reference that describe respective exemplary algorithms: “A Comparison of Event Models for Naive Bayes Text Classification” AAAI-98 Workshop on Learning for Text Categorization (Vol. 752, No. 1, pp. 41-48); and “word2vec Explained: deriving Mikolov et al.'s negative-sampling word-embedding method” Goldberg, Y., & Levy, O., arXiv:1402.3722 (2014).

After identifying and understanding the contents of the conversation, and before responding to the user demonstrating the understanding of the content of the user's statements, the computing system must detect the “tone” of the user's statements and respond using an emotional tone that is similar to or appropriate for the tone the user has conveyed. In particular, understanding and emulating the user's tone allows the computing system to demonstrate that it is aware of the user's feeling toward what is said and that understanding makes it feel the same feeling.

As an alternative to choosing from a list of available prompts, once the user's tone or other characteristic has been identified, the computing system may synthesize a new prompt using natural language generation techniques. For example, using the entity “John,” the relationship “brother,” the topic “meal,” the subtopic “dinner” and the tone “fun,” the computing system may synthesize “Sounds like your brother John and you had a fun time during dinner!” As a further alternative, the computing system may draw from an inspirational quote or mention a fact from a research study. In some versions, the prompt may also be composed using real time query of online resources. For example, the prompt can be based on the variety of information that is available on the web. If it is detected that the user is describing a topic that happened recently, the computing system can go online to news websites and generate a prompt taking these events into account. In accordance with the present invention, generating a prompt with information that is based on recent event may be more effective in grabbing the user's attention. For instance, if the name of a rock band is continuously detected as a topic, providing a real time update on that rock band may serve to draw the user deeper into the conversation. Once the mirroring prompt is administered and played to the user, the computing system continues with the normal course of interaction with the user.

In accordance with the present invention, if and when the mirroring prompt feature is activated, a sub component such as a dialogue manager or an interaction manager within the computing system may perform one or more of the analyses discussed above. Various components may work concurrently to train and/or retrain the classifier in real time, run real time analysis on the dialogue or the conversation, and retrieve or generate a mirroring prompt that serves multiple purposes, e.g., show empathy, increase adherence, etc.

In certain embodiments, an interactive session as discussed above is defined by the user freely speaking in the presence of the computing system. During the interactive session, the computing system may similarly speak back to the user and engage in an auditory conversation with the user. The computing system may intelligently adjust volume, pitch, gender, etc. of the spoken voice to as part of simulating empathy. For example, the computing system may distinguish a loud voice response from a quiet voice response. The computing system may also distinguish a rapidly spoken response from a calmly spoken response. The computing system may further distinguish an immediate response from a contemplated response. As such, the mirroring prompt may be more verbose or succinct or more high-key or low-key. When it is detected that the user is taking his or her time to answer a question, even prior to receiving a response, the computing system may ask what the user is thinking about. Accordingly, the mirroring prompt is not only relevant and indicative of identified topics and/or reflective of the ascertained tone from the user's response, but also contemplative of the user's mood, the user's habit, the user's manner, the user's style, etc.

### Types of Interactive Sessions

An interactive session is triggered when the user is presented with an activity to be performed. As described above, some exemplary activities require the user to answer a series of questions. When these types of activities are presented, the session may become “interactive” when the user provides a response. As discussed above, the inventive computing system analyzes the text of the received response and simulates conveyance of empathy to increase the user's level of engagement to a particular activity or a happiness track.

In certain other embodiments, the user communicates with the computing system via a screen and a keyboard by ways of typing and reading words on the screen. The computing system may intelligently adjust the manner in which words are displayed, such as color, font or size or incorporate pictures or short video clips as part of simulating empathy.

In certain further embodiments, when a more physical activity is presented, such as requiring the user to perform a certain action, e.g., perform an exercise, go interact with other people, etc., the performance of the activity by the user is monitored via various modules and sensors in connection with the computing system. When these types of activities are presented, the session may become “interactive” upon the computing system detecting a certain facial expression or a certain bio-physical change. For example, when the user is instructed to perform a particular exercise to help clear the user's mind, the computing system may monitor the user's heart rate and interrupt to provide an alternate activity when the user's heart rate has reached a certain threshold. Or, the computing system may monitor the user's posture and provide a guiding prompt. In these embodiments, the computing system can also simulate empathy, just as it does in an auditory or a visual conversation, by expressing a mirroring prompt that shows an understanding of the user's current feelings and/or by providing words of encouragement to show that the computing system is watching the user's performance in the shoes of the user.

As another example, when the user is performing a physical action as part of performing the presented activity, the computing system may analyze the facial expression, the voice, the gestures, etc. of the user to determine the user's mood or attitude toward the particular activity. Based on detecting certain facial expressions or hand gestures, the computing system may output a mirroring prompt. In accordance with the present invention, based on detected facial expression, the mirroring prompt may be commiserative, encouraging, sympathetic or mirroring. In other words, these additional input data from the sensors impact how the computing system determines the tone of the outputted mirroring prompt.

Accordingly, the feature of providing a mirroring prompt during an interactive session can be achieved through numerous ways. In the end, the computing system displays emotional intelligence by mirroring the user in the most appropriate way possible and such effect leads to a higher level of engagement and an increased commitment to remain engaged with the activity or track.

### Proactive Triaging

In certain other embodiments, the inventive system includes artificial intelligence sufficient to provide a “proactive triaging” ability. One of the biggest causes for a drop in the level of engagement with sustained usage of program or application such as the Program is that the user is not finding a particular activity exciting or relevant. There may be additional different reasons why a user may not find wish to further engage with an activity. In some cases, the user is partaking in an activity while internally desiring something else. Most of the time, the user would not even bother requesting for a change and simply lose interest in continuing with the program. In one or more of these cases, it may be that the user is simply preoccupied with a certain different issue without fully realizing it.

As described in greater detail herein, with such proactive triaging ability, the computing system is capable of detecting, during an activity in progress and/or during an interaction with a user, that the user is currently focusing on a topic other than the one intended by the system, or focusing on a topic that is more relevant to a different Program track or activity, and in such case, the system “proactively” suggests a suitable change to the user. Discovering the fact that the user is preoccupied with a different issue is in fact a new insight and a realization shared with the user. For instance, during execution of a particular activity within a selected Program track, the computing system detects particular user behavior, characteristics and/or user feedback indicating a necessity for proceeding with a different activity within the selected track or proceeding to a different Program track entirely and recommends a change to the user when appropriate.

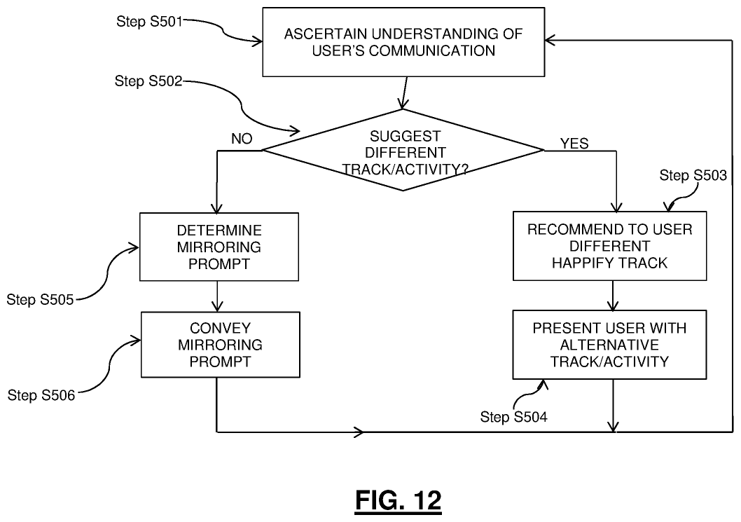

In accordance with an exemplary embodiment of the present invention, the user is engaged in an interactive session with the computing system. As shown in FIG. 12, the process for proactive triaging begins with ascertaining an understanding of the user's communication (Step S501). This step, similar to the mirroring feature described herein, entails multiple sub-steps. For example, the computing system employs techniques such as natural language classification, topic modeling, sentiment analysis, named entity extraction, emotion detection, etc. to identify and understand the contents of the user's communication. As previously described herein, the computing system may, for example, employ a trained classifier and identify one or more topics from the user's communication.

Once the computing system identifies topics from the content of the user's response, it determines whether a branching suggestion should be made (Step S502). This step also entails multiple sub-steps. For example, the computing system may employ a threshold system in which a determination as to suggesting a different track/activity is made when words suggestive of a different topic appear a certain number of times. As another example, the determination is made when none of the topics identified relates to the current activity/track. As yet another example, relevance of identified topics to the current activity/or track may be measured in a range of scale, and the branching determination is made when the relevance of the identified topics to the current activity/track is below a threshold level. As a further example, the computing system detects certain keywords that necessitate a branching suggestion. In some embodiments, the exact same set of AI engines as described above, e.g., emotion detection, topic modeling, natural language classification, etc. are used to determine whether or not the branching suggestion should be presented to the user. For example, sensors may detect certain facial expressions or gestures indicating lack or loss of interest and the computing system determines that the branching suggestion should be made. As another example, the computing system may keep a track of the progress of the user in regards to the provided activity and/or the selected Happiness track, and a branching determination is made based on the level of progress of the user. The goal of proactive triaging is that at each and every turn in the dialogue/conversation, the computing system conducts proactive triaging to re-evaluate what is the best course of interaction/treatment for the user.

If it is determined that the branching suggestion should be made, the process proceeds to step S503. In step S503, the computing system notifies the user that the user is seemed to be focusing on a topic that is different from the current activity and presents a recommendation. When the user accepts the suggestion, the computing system presents the user with alternative track/activity that has been determined as the better course of action for the user (Step S504). Thereafter, the process can be repeated to determine how well the user is interacting with the new activity/track.

If it is determined that the branching suggestion is not needed, the process proceeds to step S505. In Step S505, the computing system determines a mirroring prompt and in Step S506, the computing system conveys the mirroring prompt to the user.

In some embodiments, the proactive triaging feature is employed without the mirroring prompt feature. In certain other embodiments, the proactive triaging feature is employed concurrently with the mirroring prompt feature. In yet certain other embodiments, the mirroring prompt feature is carried out prior to the proactive triaging feature. Therefore, in some embodiments, the proactive triaging feature is the “next step” to the process of mirroring as disclosed herein. In accordance with the present invention, proactive triaging, thus, can be referred to as first, empathizing with the user and second, providing an advice or making a suggestion for a course of action to the user based on understanding of the user's emotion. More particularly, with proactive triaging, the computing system analyzes, for example, what the user has said and the manner in which it is said and provides an appropriate suggestion. In some embodiments, the computing system will not only provide a suggestion, but also explain the reasoning behind it.

An example of the proactive triaging in a conversation employed by the present invention is shown in Tables 5 and 6.

TABLE 5
Steps Leading to Proactive Triaging

| | |
|---|---|
| Computer | > What are things you can do to improve the quality |
| | of the time you spend with your children? |
| User | >> Perhaps if I stopped thinking about my debt and |
| | the possibility of filing for bankruptcy when I am with |
| | them, perhaps then I could be more present. |
| Computer | > Yes, it's normal to worry about things, but too much |
| | worry can rob us of good life experiences. |
| Computer | > Is there something else you can do to improve the |
| | quality of the time you spend together? |
| User | >> Well, I was thinking of taking them to the movies |
| | or even on a day trip up to the mountains, but every |
| | time I think of something like that I get scared about |
| | expenses and not being able to pay for it. |

TABLE 6
Proactive Triaging

| | |
|---|---|
| Computer | > What are things you can do to improve the quality |
| | of the time you spend with your children? |
| User | >> Perhaps if I stopped thinking about my debt |
| | and the possibility of filing for bankruptcy when I am |
| | with them, perhaps then I could be more present. |
| Computer | > Yes, it's normal to worry about things, but too |
| | much worry can rob us of good life experiences. |
| Computer | > Is there something else you can do to improve |
| | the quality of the time you spend together? |
| User | >> Well, I was thinking of taking them to the movies |
| | or even on a day trip up to the mountains, but every |
| | time I think of something like that I get scared about |
| | expenses and not being able to pay for it. |
| Computer | > I noticed that you mention worrying or perhaps |
| | being stressed about finances. Happify has a track |
| | called: “Dealing with Financial Stress.” |
| | Would it be useful for you to check it out? |

Initially, it should be noted from the above conversation that the computing system has employed the mirroring prompt and demonstrated human-like empathy by demonstrating an understanding tone and reflecting on the content of what the user just said, e.g., “it's normal to worry about things”. Moreover, the computing system continues the interaction and receives the user's further responses. During the course of the interaction, the computing system performs aforementioned analyses on the input data and identifies one or more words that are indicative of a different topic being mentioned repeatedly. For instance, in the above example, the computing system identifies the terms “debt,” “bankruptcy” and “expenses” that all belong to another group, e.g., “financial management”. The computing system also recognizes a negative tone in relation to the usage of these terms in the conversation. The computing system also recognizes a repetition of these terms in the conversation. At this point, as shown in Table 6, in addition to simply empathizing or showing support, the computing system proactively suggests that the user switch to a different track that is focused on financial worry.

The present digital therapeutic is designed to improve patient conditions according to one or more clinical measurements. For example, the Patient Health Questionaire-9 (PHQ-9), also called the DEP-9, is a depression scale from the Patient Health Questionaire (PHQ) containing nine questions that is used to make a depression diagnosis according to DSM-IV criteria. The PHQ-9 may also be used to track the progress of a user over time. Generalized Anxiety Disorder 7 (GAD-7) is similar to PHQ-9 but focuses on anxiety issues instead of depression and may be used similarly to diagnose and track anxiety. The digital therapeutic described herein creates physiological changes in patients that may be measured by the PHQ-9, GAD-7 and similar tools.

A number of implementations have been described. Nevertheless, it will be understood that various modifications may be made without departing from the spirit and scope of the invention. Accordingly, other implementations are within the scope of the following claims, including the following implementations, expressed as interrelated items:

### Digital Therapeutics for Migraine

The migraine digital therapeutic presented herein is a multi-week computerized behavioral therapy used to treat migraine, either directly or in connection with treating one or more mental disorder such as depression and anxiety. More specifically, the treated disorder may be one of more of major depressive disorder (MDD) and generalized anxiety disorder (GAD) and psychiatric disorders related thereto. The symptomology or comorbidity relationship between migraine and any psychiatric disorders may be indirect. That being said, treatment of disorders such as MDD and/or GAD have been shown to impact migraine symptoms or side effects of migraine treatments such as CGRP receptor antagonists.

The digital therapeutic presented here is based on the principles of cognitive behavioral therapy (CBT) and the related disciplines of acceptance and commitment therapy (ACT) and positive psychology. The digital therapeutic is intended to impact migraine symptoms and side effects by treating MDD and/or GAD. This MDD/GAD treatment is based upon correcting maladaptive patterns of cognition and helping patients engage in healthier behaviors.

### Generally Useful Definitions

Cognitive Techniques. These interventions emphasize making changes to maladaptive thinking patterns that maintain psychiatric conditions. Cognitive techniques might include: evaluating thoughts based on evidence, conducting experiments to gather new evidence to evaluate a thought, and thinking through the probability of negative outcomes. For example, cognitive techniques for MDD and/or GAD often include challenging unhelpful positive beliefs about worry, e.g., superstitious beliefs that worrying prevents negative outcomes).

Behavioral Techniques. Behavioral interventions are grounded in learning theory (operant and/or classical conditioning) and emphasize reward, punishment, habituation and extinction. Examples include exposure to feared stimuli or sensations to reduce anxiety and engagement in valued activities to reduce depression by increasing exposure to rewards.

Positive Psychology. Interventions from positive psychology include any skill to help patients notice pleasant experiences or emotions, focus on positive aspects of their lives, or improve functioning above baseline or statistical normality. Examples include gratitude exercises, imaging future events with joy and optimism, counting blessings, etc.

Psychoeducation. Psychoeducation provides important psychological information to the patient, typically about the disorder being treated or about the techniques that will be used within the treatment. In the digital therapeutic presented herein, we provide psychoeducation about functions of worry, mindfulness and acceptance as an alternative to worry, particularly in the context of migraines, how savoring and engagement in valued activities can be helpful, etc.

Daily Monitoring. This involves daily recordings of important symptoms or behaviors. In the present digital therapeutic product, a daily worry diary for much of the multi-week program may be included. It is designed to gather information about the patient's worry patterns, e.g., triggers, common worry topics), as well as to increase the patient's awareness of when they are engaging in worry.

Mindfulness. Mindfulness interventions are based on focusing attention on something specific while withholding judgements about whether it's good or bad. The present digital therapeutic provides both formal mindfulness meditation exercises (for example, mindful breathing, mindful eating, mindfulness to emotion) and more pragmatic or informal mindfulness exercises (for example, mindfulness to daily activities such as focusing on the feeling of wind on your face while walking to work or school, or mindfulness to daily chores like laundry or dishwashing).

Acceptance. Acceptance-based interventions involve shifting behavior to be effective and pragmatic by acknowledging the truth about one's current situation and working within those constraints. The goal is to reduce unnecessary suffering due to engaging in ineffective behaviors. In this digital therapeutic, acceptance-based activities may include acceptance of difficult emotions such as anxiety, acceptance of uncertainty regarding the future, or acceptance of the possibility of future problems or negative outcomes.

Problem solving. These interventions provide basic skills in problem solving which patients may have failed to develop because they were either too anxious and avoidant to learn them or because they suffer from a neurocognitive problem that makes learning these kinds of skills difficult. Either of these circumstances may be directly or indirectly related to their migraine condition. The present digital therapeutic provides important skills for solving problems as an alternative to worrying about them. The patient may work to solve current problems by identifying and defining the specific problem to be solved, thinking through several alternative options available to them in a given situation, and choosing one that is good enough, even if it does not guarantee a successful outcome. For example, a patient who is struggling at work will learn to define the specific issue, e.g., workload is too heavy), generate several alternative solutions, e.g., communicate with supervisor about ways to manage or decrease workload; hire a new employee; quit job), and select and implement the most promising solution, e.g., communicate with supervisor).

Social skills training. These interventions may involve explicitly helping the patient practice interactions with others. These techniques are particularly useful for patients with high anxiety or neurodevelopmental problems.

Relaxation. The intervention may involve specific methods for reducing physiological arousal such as guided imagery or physical activities to reduce muscle tension. The digital therapeutic may include written instruction and audio recordings to help the patient develop skills in progressive muscle relaxation where they are instructed to tighten and release all major muscle groups in a specific order.

Goal setting. Goal setting interventions help patients to define concrete, specific, and achievable behavioral goals that are consistent with their values. For example, these interventions may help a patient to identify physical fitness as an important value and to set a corresponding goal of walking outdoors for 30 minutes each day.

Termination. These interventions focus on reviewing material learned to consolidate gains and prevent relapse. Making specific plans to cope with increased worry in the future is one example of this kind of intervention that is included in migraine digital therapeutic.

The present migraine related digital therapeutic delivers therapy in a sequence of modules of neurobehavioral interventions, patient education, and skill-building. It may be implemented in the form of a mixture of text, videos, quizzes, and interactions with a conversational artificial intelligence (AI) chatbot. The therapy is delivered via a software application intended to be used on a patient's mobile device or any other computerized device, the software application accesses additional software associated with the digital therapeutic through a web-based portal or similar network access point.

Besides the patient's application interacting with additional software as part of a patient's treatment program, clinicians have access to a clinician dashboard that shows, among other things, how a particular patient is utilizing and engaging with the application, i.e., the digital therapeutic. Further, this dashboard provides access to relevant patient information for each patient regarding which a given clinician has authorization, such that the clinician may switch between patients as necessary. A fuller device description is provided herein.

The present digital therapeutic is a personalized treatment for patients suffering from migraine with related mental health issues such as MDD and GAD. The mental health component may be a directly or indirectly related to the migraine diagnosis or treatment or even completely unrelated thereto. It is based on empirically supported interventions from CBT among other neurobehavioral interventions. CBT is the term used for a group of psychological treatments supported by several decades of scientific evidence. Such therapies are sometimes short-term treatments that focus on teaching specific skills to a patient and have been shown to be effective in randomized clinical trials for MDD and/or GAD. Many such randomized trials utilized face-to-face delivery of treatment by a trained clinician. A feature of the present digital therapeutic is that such face-to-face interventions have been adapted to work in a digital format, i.e., utilizing software and networked connections between the patient and at least portions of the digital therapeutic software.

Further, the CBT-related aspects of the present digital therapeutic has been enriched with content from other neurobehavioral and related interventions including acceptance and commitment therapy (ACT) and positive psychology. ACT is a form of psychotherapy that has been extensively researched. It emphasizes interventions that use acceptance and mindfulness strategies while emphasizing the commitment to values and behavior-change strategies to increase psychological flexibility. positive psychology is the scientific study of the positive aspects of the human experience that make life worth living. Content from these related disciplines is reflected in some of the specific interventions delivered through the present digital therapy and are discussed further with regard to therapeutic modalities, where additional details are provided.

The migraine digital therapeutic can be personalized to the patient in ways that do not compromise the fidelity of the treatment and, in fact, are designed to markedly increase the efficacy thereof. Personalized mechanisms are discussed in details in sections below concerning the device overview and device personalization for the migraine digital therapeutic.

GAD has historically been more challenging to treat than other anxiety disorders, with a high number of patients continuing to report clinically significant symptoms after treatment (Borkovec & Ruscio, 2001). CBT for GAD typically emphasizes self-monitoring of worry (a cognitive process) and anxiety (an emotion) to increase early awareness of anxiety cues and behaviors, followed by skills to manage worry and anxiety spirals. These skills may include: changing thoughts and beliefs, relaxation training, scheduled “worry time,” planning pleasant activities, and controlled exposure to thoughts and situations that are being avoided. The migraine digital therapeutic described herein has incorporated each of these skills into its digital interventions. The therapeutic may also include acceptance-based approaches to increase mindful awareness and engagement in valued actions as well as skills for increasing tolerance of uncertainty. The digital therapeutic product represents an integrative approach because it appeals to the largest population without sacrificing safety or efficacy.

A substantial portion of the U.S. population are affected by MDD and/or GAD. Further, it is unsurprising that an even higher proportion of persons suffering from frequent migraines are also affected by MDD, GAD and/or related mental health issues. There is a clear need to provide therapeutic intervention for MDD and/or GAD on a large scale, and digital solutions can meet this requirement. 81% of Americans own a smartphone, and nearly 75% own a desktop or laptop computer (Pew Research Center, 2019). Psychological interventions that are delivered using these devices can help to increase access to care, which is a significant issue in the US with many depression and anxiety patients receiving suboptimal care or no care at all and can also help overcome barriers like the perceived stigma associated with mental illness. Digital interventions can help make effective treatments available more widely. There has been a substantial increase in research on such interventions, and the available evidence shows that these kinds of interventions can help specific disorders, even in patients with co-occurring medical conditions.

In addition to high prevalence in the general population, depression and anxiety disorders (like other mental health problems) are even more prevalent in people suffering from chronic medical conditions such as migraine. A global study of 42 countries concluded that several highly prevalent chronic physical conditions are significantly associated with depression and/or anxiety and having just one condition increased the odds of depression and/or anxiety symptoms by almost twofold. A person suffering chronic migraine, i.e., 15 or more headache days per month, has between 30 and 50% chance of depression. The rates of anxiety in chronic migraine suffers is even greater, estimated above 50%. Much of the anxiety felt is about when their next migraine attack will occur and how it will affect their life.

The present migraine digital therapeutic treats MDD and/or GAD with CBT interventions that modify or reverse maladaptive patterns of cognition and behavior. The specific targets for MDD and/or GAD include the following:
faulty estimates about the likelihood of negative events,
distorted views about one's ability to cope with difficult events,
poor problem-solving skills,
positive beliefs about worry, e.g., as a way of preventing, problem-solving, or coping with negative events),
intolerance of uncertainty, and
cognitive, behavioral, and experiential avoidance.

At present, the digital therapeutic product described herein includes 112 total interventions aimed at modifying these processes. Additional details about the specific therapeutic modalities that are represented and included in the migraine digital therapeutic are provided below.

The present migraine digital therapeutic is a multi-week therapy that, for example, may be implemented as an 8-week digital therapeutic used to treat migraine, symptoms of migraine or side effects resulting from pharmaceutical treatment of migraine. Principles of CBT is an important component of the digital therapeutic, as are principals of ACT and positive psychology.

CBT is typically delivered by a clinician in a one-on-one format, although group formats are also sometimes used. Standard exposure of a patient to CBT usually occurs in weekly sessions over 8-14 weeks. CBT can be conceptualized as a skills-based treatment that delivers proven behavioral and cognitive treatment strategies. A ‘skills-based treatment’ may be contrasted, for example, with an insight-oriented treatment.

Among many other advantages of a digital therapeutic is the flexibility of ‘dispensing’ and ‘dosing’ treatment. That is, the digital and networked nature of the treatment means that there is no need to schedule treatment(s) based on availability of a healthcare professional (HCP) or other factors, nor does there need to be any considerations of travelling to the HCP's office. It has been found that a dosing of two digital therapeutic interventions per day is easily achievable by the average patient. Other than in an in-patient setting, such a therapy frequency is completely unattainable. This being the case, a battery of CBT substantially shorter than the typical 8-14 weeks may be achieved. The present migraine digital therapeutic may have a duration of between about four weeks and fourteen weeks, including durations of about four weeks, six weeks, eight weeks, ten weeks, twelve weeks and fourteen weeks. However, the product's design allows flexibility to accommodate the needs of the patient, e.g., for missed interventions. Patients may be provided access to the migraine digital therapeutic for more weeks than typically required to complete to provide additional accommodation.

The availability of the digital therapeutic may be set to end automatically based on the start date, i.e., the date the patient creates an account and begin the treatment. A variable number of interventions may be unlocked each day. For many patients, two interventions per day seem to encourage ongoing engagement with the therapy. One, three or four interventions are, however, both feasible and even advisable for some patient groups. Obviously, if the time commitment and/or complexity of interventions were adjusted significantly upward or downward, this would have an impact on the appropriate dosage per day for the average patient. In the event that a patient does not complete the total number of interventions set for a given day, they may be required to do so before new interventions are unlocked. The ‘flow’ and order of interventions for a digital therapeutic has the potential to significantly impact the efficacy of the therapy.

The number of interventions in a particular digital therapeutic, as well as the order and flow of these interventions, will be an important factor in the efficacy of a digital therapeutic. For example, completion of two interventions per day for eight weeks has been measured as delivering efficacy for indications such as MDD and GAD. The entire treatment course under these circumstances would involve completion of one hundred and twelve interventions. Again, the potential exists for extending the duration of treatment so a patient can complete the full course of treatment. In addition, such flexibility as permitting patients to make up one intervention per day, for a total of three interventions in a day, may be used to keep a patient to schedule.

The migraine digital therapeutic delivers neurobehavioral interventions in a sequence of four modules: (1) learn about worry (2) reduce suffering (3) increase joy and meaning, and (4) maintain progress. See, e.g., FIG. 1. These interventions are delivered in four key media formats: 1) conversation guided by an AI chatbot, 2) audio recordings, 3) video recordings, and 4) quizzes. each module is described in detail hereinbelow.

The present digital therapeutic may request patients complete a daily worry diary as part of the therapeutic intervention. This diary may be completed through interactions with the AI chatbot. An advantage of delivering the worry diary via the AI chatbot is that it allows patients to ask questions and receive guidance if needed. This patient may monitor and record worry episodes and topics and situational and internal triggers for worry and associated emotions. It is intended to help give the patient a clear picture of their worry and increase the patient's awareness of when they are engaging in worry—which is particularly important because worry is a covert event. As patients become more familiar with their worry patterns, they will also learn to categorize each thought as 1) worries about current problems that may respond to in-the-moment problem-solving or 2) worries about potential problems that may never actually come to pass and will be managed with acceptance and mindfulness. Insights from self-monitoring may be used later in the treatment.

Psychoeducation is also provided throughout all modules of the treatment and may include information about treatment rationale, common pitfalls, and scientific models about how thoughts, behaviors, and emotions influence migraine, migraine symptoms, migraine treatment side effects, GAD and MDD.

The presently described migraine digital therapeutic may be personalized to address key interest areas, increase engagement and accomplish other important efficacy goals, in several ways:
1. Information the patient provides through onboarding questionnaires and responses to the AI chatbot is saved, to be used later in the treatment. This information may be referred to as “life graph variables” that can be accessed to give the AI chatbot dialogues a more personalized feel. For example: if a patient tells the AI chatbot that he has a dog, she will ask the dog's name and store it under the life graph variable “dog_name.” The chatbot may then use that name in any future task. If the patient completes an intervention two weeks later and suggesting that he go for a walk is appropriate, the AI chatbot may reference that name in the suggestion in the following way, “Perhaps you could take a walk with your dog Lolly.” If the life graph variable dog_name is empty, Anna will say, “Perhaps you could take a walk.”
2. The patient may be provided with the option of completing the MDD and/or GAD treatment with an emphasis on family concerns, career stress, or physical wellness, as further discussed below. If they choose one of these options, the treatment content may be modified with examples relevant to that area of interest. It is not necessary to change the number of interventions or the therapeutic modality of the interventions to achieve this. Rather, interventions may simply be adapted to better reflect the patient's interest. This approach utilizes Program's Indication Treatment Sequence described below. Briefly, this sequence specifies each intervention's therapeutic modality and the order in which those interventions are to appear. For example, day 19 of the treatment has one intervention that delivers a brief mindfulness exercise and one intervention that delivers psychoeducation regardless of whether the patient is completing the version that emphasizes family concerns, career stress, or physical wellness. On day 19, the mindfulness exercise content is adapted slightly, and examples within the psychoeducation intervention are modified based on which experience is offered, but the core therapeutic elements remain the same.
3. The migraine digital therapeutic can adapt to treat MDD and/or GAD in specific populations, notably in people suffering from chronic medical conditions, such as migraine, asthma or psoriasis. For these cases, the digital therapeutic may enrich psychoeducation with information about the medical condition and MDD and/or GAD and ensures that all examples and suggested activities are appropriate for people suffering from that condition. For example to adapt for a patient with migraine, the digital therapeutic may replace recommendations to go for a run with recommendations meditate instead. Changes may follow an indication treatment sequence, discussed further below, and be personalized to ensure the suggestions are appropriate for the intended subpopulation.

The migraine digital therapeutic presented herein is a software application intended to offer at-home treatment for migraine, GAD and/or MDD in an engaging, user-convenient format as a prescription or over the counter digital therapy. This design is expected to result in a safe, effective, and convenient treatment option that supports patients' compliance and offers a favorable risk-benefit profile.

The Instructions for Use (IFU) for the present therapeutic may note that it should be used under the supervision of a licensed Health Care Provider (HCP) and it is not meant to be a substitution for any treatment medication. The IFU will also include product specific warnings and contraindications.

The migraine digital therapeutic may include a variable number of neurobehavioral modules, with the specific number of modules determined by a number of factors. Similarly, the number of interventions per module is also an important factor in designing the digital therapeutic. Important factors in making these determinations are efficacy of the treatment and retaining engagement of the patient for the entire course of treatment. Experiments involving actual patients may be conducted with varying numbers of modules and interventions may be utilized to achieve efficacy and patient retention. Experimental data has been collected to general uses of digital therapeutics as well as toward specific indications treated by such therapeutics, e.g., migraine, MDD and GAD. Further, since many interventions and even whole modules are potentially useful across indications and mental health disorders, much knowledge has been gathered by developers of the present digital therapeutic that is useful in determining how to maximize efficacy and patient retention as well as in designing experiments of the type discussed. The present migraine digital therapeutic may utilize four neurobehavioral intervention modules and provides performance feedback to patients and clinicians.

Looking to a digital therapeutic focused on treating GAD, the first module is organized to achieve three treatment goals:
1. To educate patients on key concepts related to worry and anxiety (psychoeducation)
2. To increase patients' awareness of relationships among thoughts, actions, and emotions related to worry (daily monitoring)
3. To improve patients' skills for identifying and targeting current problems, defining goals, and brainstorming and implementing possible solutions (problem-solving)

The first module focuses on introducing patients to the treatment program and setting the right framework for success. It begins with education about GAD symptoms followed by daily monitoring of thoughts, actions, and emotions related to GAD. The educational content is focused on the nature of anxiety and worry and common misconceptions about the value of worry. For example, many people with MDD and/or GAD believe that worrying protects them or that worry is required if you love someone. Self-monitoring involves paying attention to worry episodes and topics along with situational and internal triggers and associated emotions. Self-monitoring is intended to help give the patient a clearer picture of their worry and increase the patient's mindfulness when they are engaging in worry, which is particularly important because worry is a covert event. It helps patients see how the diagnosis affects them as individuals. Next, problem-solving is introduced to ensure the patient has a viable alternative to worry.

As patients become more familiar with their worry patterns, they will also learn to put worries into two categories: 1) worries about current problems that may respond to in-the-moment problem-solving and 2) worries about potential problems that may never actually come to pass and will be managed with acceptance and mindfulness. Problem-solving skills are introduced to address worries in category 1. Many individuals with GAD see problems as a threat of failure, avoid facing current problems, or lack practical problem-solving skills. These skills include correctly identifying current problems, defining goals, and brainstorming and implementing possible solutions.

Interventions in Module 2 for GAD may be organized to achieve three treatment goals:
1. To educate the patient on critical concepts related to mindfulness and acceptance
2. To increase nonjudgmental awareness and acceptance of experiences in the present moment
3. To increase psychological flexibility and willingness to tolerate uncomfortable experiences and emotions

Mindfulness and acceptance-based techniques help patients to replace future-focused worry and anxiety with nonjudgmental awareness and acceptance of experiences in the present moment. It includes increasing psychological flexibility and willingness to tolerate uncomfortable experiences and emotions, including the anxiety and uncertainty inherent in life. For example, nobody knows for sure if they will have a job in two weeks and no worrying can change that. Therefore, patients are encouraged to observe and sit with the uncomfortable emotions and sensations associated with that reality. This module will include psychoeducation about mindfulness and acceptance, along with formal and informal mindfulness exercises. Mindfulness and acceptance-based techniques are useful for many purposes, including MDD and/or GAD.

Interventions in Module 3 may be organized to achieve three treatment goals:
1. To help patient to identify their values and the ways in which worry and anxiety interfere with valued activities
2. To help patient set goals related to their values
3. To increase patient's mindful engagement in valued activities despite the anxiety

Module 3 emphasizes increasing engagement in activities motivated by the patient's values, rather than by anxiety or worry. Worry and anxiety often interfere with patients' engagement in valued activities. Even if they are going through the motions of participating in valued activities, the worry and anxiety may distract mindful focus on these activities and reduce meaning and satisfaction. This module's primary goal is to help patients move from a place where their activities are dictated by avoidance of worry, anxiety, or feared negative outcomes to a place where they mindfully and fully engage with valued activities despite anxiety. This module includes exercises to help patients to identify their values and make specific plans to engage mindfully in values-driven activities and goals, despite anxiety.

Interventions in Module 4 are organized to achieve three treatment goals:
1. To consolidate gains and maintain progress into the future
2. To review key concepts
3. To make a plan for how to cope with future worries and anxiety

The final module is focused on consolidating what the patient has learned and maintaining improvement in symptoms. Key interventions emphasize positive psychology to ensure a focus on continued growth and flourishing and planning & termination interventions. Key themes from psychoeducation are reviewed, and skills are practiced. Patients are guided through creating a list of helpful knowledge and skills that they can review in the future if anxiety increases. This section provides a helpful framework for thinking about relapse as a challenge that can now be met with greater success than patients would have had before treatment with migraine digital therapeutic.

A migraine focused digital therapeutic may incorporate performance feedback to both the prescribing clinicians and to the patients using the product.

A validated self-report measure of GAD symptoms, The Generalized Anxiety Disorder Scale-7 (GAD-7), is administered by the product as part of the treatment. The GAD-7 is one of the most frequently used diagnostic self-report scales for screen, diagnosis and severity assessment of anxiety disorder and it was developed by Drs. Robert L. Spitzer, Janet B. W. Williams, Kurt Kroenke and colleagues. Patients using the presently described migraine digital therapeutic are requested to complete GAD-7 scale during their treatment. For example, a GAD-7 assessment may be done prior to treatment as a baseline and then every week or every two weeks throughout treatment.

GAD-7 scores of 5, 10, and 15 are reported to the patient as indicating mild, moderate and severe anxiety respectively. Raw scores may be presented to the patient with additional text to explain what the score means. The migraine digital therapeutic may use the standard cutoff scores recommended in the scoring manual. These scores may be cumulatively graphed and presented to patients immediately following each completion of the measure, allowing them to easily track and understand their progress over time. Data from the GAD-7 may also sent to the prescribing physician. Feedback to the prescribing clinician may be delivered through a secure clinician portal. Clinicians will be able to log into the portal at any time to see compliance statistics and the performance metrics described above. Patients using the product will likely be informed that this information is being shared with their clinician.

The migraine digital therapeutic may include a conversational AI chatbot feature designed to mimic human interaction. The chatbot may be referred to utilizing a human name, e.g., “Anna” or the like, so as to give it a more personalized touch, but it is clearly stated to the patient that this is a computer, not a real person. Anna may guide the patient's engagement with each intervention via a conversational dialog that responds to the patient's text. In many cases, this may involve greeting the patient and collecting information. Anna employs a mix of instruction and feedback that includes open-ended questions, multiple-choice options, and clarifying examples to guide the patient.

In addition to the clinical interventions delivered daily, polls and games are designed to make the experience more enjoyable for the patients. These engagement features are not necessarily considered part of the dosing or the therapy. The patients are not required to interact with these features but may earn a “gold medal” if they complete all required interventions in a module within the prescribed time frame. Those who require extra time to complete a module may receive a silver medal. Also, the migraine digital therapeutic may be designed with a community feature to share activities the patients have completed, newsletters, and infographics containing relevant information about mental health.

The current migraine digital therapeutic was designed to be personalized without compromising its clinical efficacy. The treatment may be set to automatically adapt based on one of a plurality of areas of interest chosen by the patient. It also includes minor personalization elements, like remembering the names of important people in the patient's life, through the chatbot Anna. The migraine digital therapeutic intervention can be adapted for work with a specific subpopulation of people suffering from migraine, such as those who have GAD, MDD or particular side effects from a migraine medication. The method for accomplishing these kinds of personalizations without compromising the treatment's clinical efficacy is described in detail below. The sections may provide detailed explanations of personalization and justification for its need in migraine digital therapeutic.

The migraine digital therapeutic may deliver brief (10-20 minute) daily interventions based on CBT and enriched with techniques from positive psychology and ACT. Each of these daily interventions can be categorized into the specific modality it represents. The developers have identified 12 therapeutic modalities into which psychological interventions can be classified. Examples of therapeutic modalities include mindfulness and behavioral interventions, with three examples, each of the specific interventions that would fall under each modality. The migraine digital therapeutic interventions may fall within the following therapeutic modalities: psychoeducation, monitoring, mindfulness, relaxation, behavioral interventions, acceptance interventions, problem-solving, positive interventions, and termination. This system of labeling each intervention with its appropriate therapeutic modality, among other functions, helps link each intervention to the scientific literature supporting its efficacy.

Activities, Tracks, Interventions, Modules and Interventions

| Activity | | Activity | | Level |
|---|---|---|---|---|
| ID | Skill | Type | Activity Name | (1-5) |
| A-01 | Aspire | Essay | My victorious self | 1 |
| A-02 | Aspire | Essay | My core values | 3 |
| A-03 | Aspire | Essay | What's My Why? | 1 |
| A-04 | Aspire | Plan-Do | I think I can | 2 |
| A-05 | Aspire | Essay | Find meaning in the mundane | 2 |
| A-06 | Aspire | Essay | Setbacks and steps forward | 4 |
| A-07 | Aspire | Essay | Pursue meaning | 3 |
| A-08 | Aspire | Plan-Do | Create meaning | 5 |
| A-09 | Aspire | Plan-Do | Spend on what matters | 4 |
| A-10 | Aspire | Essay | I have a goal | 5 |
| A-11 | Aspire | Essay | I'm looking forward to . . . | 1 |
| A-12 | Aspire | Plan-Do | Developing and | 2 |
| | | | Using My Strengths | |
| E-01 | Empathize | Essay | Give myself a break | 1 |
| E-02 | Empathize | Essay | Walking in their shoes | 2 |
| E-03 | Empathize | Essay | Whats my positive impact? | 1 |
| E-04 | Empathize | Essay | Weird . . . why'd they do it? | 2 |
| E-05 | Empathize | Plan-Do | Get to know someone | 3 |
| E-06 | Empathize | Essay | Empathize with a | 3 |
| | | | different viewpoint | |
| E-07 | Empathize | Essay | Not cool-why'd they do it? | 3 |
| E-08 | Empathize | Essay | Empathize when you disagree | 4 |
| E-09 | Empathize | Essay | That hurt! What | 5 |
| | | | made them do it? | |
| E-10 | Empathize | Plan-Do | Help someone | 4 |
| E-11 | Empathize | Essay | There's No One Like Me | 4 |
| E-12 | Empathize | Essay | Empathize when | 5 |
| | | | you're resentful | |
| E-13 | Empathize | Do | Create a micro- | 2 |
| | | | moment of connection | |
| G-01 | Give | Plan-Do | Give a small gift | 1 |
| G-02 | Give | Do | One day, 5 nice things | 4 |
| G-03 | Give | Plan-Do | Make someone smile | 1 |
| G-05 | Give | Plan-Do | Spend time on a | 2 |
| | | | valuable activity | |
| G-06 | Give | Plan-Do | Spend $$ on a | 3 |
| | | | shared experience | |
| G-07 | Give | Plan-Do | Celebrate someone's | 4 |
| | | | good news | |
| G-08 | Give | Essay | Forgive an annoyance | 4 |
| G-09 | Give | Plan-Do | Spend time with someone | 3 |
| G-10 | Give | Essay | Forgive an offense | 5 |
| G-11 | Give | Plan-Do | Volunteer | 5 |
| G-12 | Give | Plan-Do | Donate $$ for a cause | 5 |
| R-01 | Revive | Plan-Do | Sleep: Brighten Your | 1 |
| | | | Wake-Up Routine | |
| R-02 | Revive | Audio | Sleep: Relax with | 2 |
| | | | Autogenic Training | |
| R-03 | Revive | Plan-Do | Sleep: Wind Down | 3 |
| R-04 | Revive | Plan-Do | Sleep: Optimize | 4 |
| | | | Your Sleep Schedule | |
| R-05 | Revive | Plan-Do | Body/Fitness: Take a Stand | 1 |
| R-06 | Revive | Do | Body/Fitness: Breathe Deeply | 1 |
| R-07 | Revive | Plan-Do | Body/Fitness: | 2 |
| | | | Make Time to Move | |
| R-08 | Revive | Plan-Do | Body/Fitness: Feel the Stretch | 2 |
| R-09 | Revive | Plan-Do | Body/Fitness: | 3 |
| | | | Build Up Your Core | |
| R-10 | Revive | Plan-Do | Body/Fitness: | 4 |
| | | | Commit to a Class | |
| R-11 | Revive | Plan-Do | Body/Fitness: Rev | 4 |
| | | | Up Your Resistance | |
| R-12 | Revive | Plan-Do | Body/Fitness: | 5 |
| | | | Commit to a Fitness Routine | |
| R-13 | Revive | Do | Nutrition: Boost | 1 |
| | | | Your Water Intake | |
| R-14 | Revive | Plan-Do | Nutrition: Start Your | 2 |
| | | | Day with Protein | |
| R-15 | Revive | Plan-Do | Nutrition: Eat More | 2 |
| | | | of the Good Stuff | |
| R-16 | Revive | Plan-Do | Nutrition: Start a | 3 |
| | | | Healthy Snack Habit | |
| R-17 | Revive | Plan-Do | Nutrition: Meal- | 4 |
| | | | Prep Makeover | |
| S-01 | Savor | Plan-Do | Savor the small stuff | 1 |
| S-02 | Savor | Do | Body scan meditation | 1 |
| S-03 | Savor | Plan-Do | Savor together | 2 |
| S-04 | Savor | Essay | Savor a memory | 2 |
| S-05 | Savor | Do | Avoid overthinking | 3 |
| S-06 | Savor | Essay/Do | Basic meditation | 3 |
| S-07 | Savor | Plan-Do | Moving meditation | 3 |
| S-08 | Savor | Plan-Do | All-day savoring | 4 |
| S-09 | Savor | Essay | Reframe negative thoughts | 4 |
| S-10 | Savor | Plan-Do | Walking meditation | 4 |
| S-11 | Savor | Plan-Do | Organize a savoring event | 5 |
| S-12 | Savor | Essay/Do | Living in the moment | 5 |
| S-13 | Savor | Do | Loving-Kindness Meditation | 3 |
| T-01 | Thank | Essay | Weekly Gratitude check-in | 2 |
| T-02 | Thank | Plan-Do | A week's worth of | 2 |
| | | | thanks (about a person) | |
| T-03 | Thank | Plan-Do | Deliver a week's | 3 |
| | | | worth of thanks | |
| T-04 | Thank | Essay | Thx Thx Thx | 1 |
| T-05 | Thank | Essay | What am I proud of? | 3 |
| T-06 | Thank | Do | Thank you note | 4 |
| T-07 | Thank | Do/Plan-Do | Deliver a thank you note | 5 |
| T-08 | Thank | Plan-Do | I'm thankful, let's talk! | 4 |
| T-09 | Thank | Essay | Today's grateful moment | 1 |

Exemplary text and an explanation as to psychological theory underlying the activities is presented below for each activity:

| Activity | | |
|---|---|---|
| ID | “You Decide How” Text | Why It Works |
| A-01 | Imagine everything | Research shows that |
| | you would have | imagining a better future |
| | hoped to happen in | can actually |
| | the next few years | make us happier-today. It |
| | actually DID | clarifies our priorities and |
| | happen. Meet your | boosts our optimism. In a |
| | “Victorious Self.” | 2010 study, subjects who |
| | Think about your | imagined a “best possible |
| | future self in two, | self” for one minute |
| | five, 10 years- | and wrote |
| | whatever works for | down their thoughts |
| | you. Imagine your | generated a significant |
| | life from all angles: | increase in |
| | your friends, | happiness. The |
| | where you live, what | researchers also |
| | you do, how | concluded that the |
| | you feel about yourself, | exercise |
| | and how you've changed. | increased the likelihood |
| | Write specific details | for someone to |
| | about what | expect a positive |
| | you've imagined and | future. If we feel optimistic about |
| | how it feels, | our goals, we're more |
| | touching on your | likely to invest the effort in |
| | personal life, | reaching them. |
| | relationships, and | When we imagine |
| | accomplishments at | and write about |
| | home and at work. | succeeding at the goals |
| | Be both realistic- | we've set for ourselves, it leads to |
| | and optimistic! | increases in well-being |
| | | and even physical health. In a |
| | | study asking participants to |
| | | write descriptions of the best |
| | | possible version of their future |
| | | selves for 20 minutes, four days in |
| | | a row, researchers found |
| | | that when compared with other |
| | | writing prompts, this |
| | | exercise was associated with |
| | | positive mood and enhanced |
| | | well-being three weeks later, and a |
| | | decreased rate of illness |
| | | five months later. In a variant on |
| | | this exercise by Sheldon |
| | | and Lyubomirsky, people who |
| | | visualized and wrote about |
| | | their best possible selves over four |
| | | weeks also showed |
| | | higher levels of motivation and |
| | | interest than those who were |
| | | simply asked to write about life |
| | | details. |
| | | Visualizing a future best self can |
| | | be a powerful catalyst for |
| | | change. It can raise our |
| | | expectations for the future, |
| | | allowing |
| | | us to break out of shackling beliefs |
| | | and set us in motion to |
| | | deliver on great things for our |
| | | future selves. |
| A-02 | Defining your values will | Defining your values will help you |
| | help you | define your life. If you |
| | define your life. If you take | take the time to really think about |
| | the time | what's important to you, |
| | to really think about what's | you'll be able to face life's |
| | important | challenges with increased |
| | to you, you'll feel more | confidence. And if you take the |
| | committed to | time to write down your |
| | living them, and be able to | values, you'll feel more committed |
| | face life's | to living them. |
| | challenges with increased | Studies found that when we engage |
| | confidence. | in activities that are |
| | Take a few minutes to | meaningful to us, we're more |
| | write down | inspired, satisfied, motivated |
| | your values. At your best, | and happy. We're more self- |
| | what kind | confident, we have a greater |
| | of person are you? What | sense of purpose, and we feel more |
| | positive | connected. And, we're |
| | traits would you like to | better able to handle life's |
| | work towards | challenges. |
| | developing? To get you | Once we know our values, we have |
| | thinking, here | a valuable checklist for |
| | is a short list of common | goal-setting. When our goals are in |
| | virtues, but | sync with our values, |
| | feel free to come up with | we're likely to pursue them with |
| | your own: | more purpose, more |
| | confidence, creativity, | satisfaction and more success. |
| | education, | Research that meaning in life |
| | faith, family, friends, | correlates positively to mood |
| | fulfillment, fun, | stability and sociable behavior. |
| | happiness, health, | Meaning at work seems to |
| | integrity, intimacy, | inspire motivation and |
| | kindness, peace of mind, | engagement. Individuals who feel |
| | status, | their work relates to a higher |
| | success, wealth, | purpose, for example, are |
| | wisdom . . . to name a | more satisfied at work and also |
| | few. | devote more effort to their job. |
| A-03 | Think about the activities | Studies have found that when we |
| | in your life | engage in activities that |
| | that are the most | are meaningful to us, we're more |
| | meaningful to you- | inspired, satisfied, |
| | what motivates you, | motivated and happy. We're more |
| | excites you, moves you? | self-confident, we have a |
| | If you don't know, and that | greater sense of purpose, and we |
| | in itself is | feel more connected. |
| | one of your concerns, take | We're better able to handle life's |
| | a look at | challenges. |
| | the categories below and | Research shows that meaning in |
| | pick two or | life correlates positively to |
| | three that fit: Parenting, | mood stability and sociable |
| | Family, | behavior. Meaning at work |
| | Friendships/Socializing, | seems to inspire motivation and |
| | Education/Personal | engagement. Individuals |
| | Growth, Career, | who feel their work relates to a |
| | Recreation, | higher purpose, for example, |
| | Spirituality/Religion, | are more satisfied and devote more |
| | Physical Health, Helping | effort to their job. |
| | Others. | |
| | Now write a few thoughts | |
| | about how | |
| | these topics are meaningful | |
| | to you and why. | |
| A-04 | Think of something you'd | Professors Edwin Locke and Gary |
| | love to | Latham have studied |
| | achieve by the end of the | goal-setting methods for decades. |
| | week- | In numerous studies, |
| | something that matters to | they've discovered the most |
| | YOU (not | successful goals are those that |
| | something your partner or | are both “challenging and |
| | boss or | specific”-in other words, they |
| | friend wants you to do)-or | should be ambitious and they |
| | a task | should be measurable. |
| | you've been avoiding. It | Another study led by Bruce |
| | could be | Headey shows that our “choice” |
| | reconnecting with an old | of goals also plays a role in our |
| | friend or | subjective well-being. Goals |
| | cleaning out your garage. | that are non-competitive |
| | Now jump ahead and | (commitment to relationships, |
| | imagine that | helping others, community) |
| | you've just completed your | promote our life satisfaction |
| | goal. How | whereas competitive goals (job |
| | is it making a difference in | advancement, making more |
| | your life? | money) are actually detrimental to |
| | What's the feeling of | our satisfaction with life. |
| | accomplishment like? | Focusing on the process, rather |
| | Write down your short- | than the outcome, can help |
| | term goal and | you achieve your goals. Research |
| | your reflections about | by Pham and Taylor |
| | completing it. | found that students who visualized |
| | Then jot down a few | themselves studying for |
| | ways-“baby | a test performed better than |
| | steps”-you might actually | students who visualized |
| | start to get | themselves doing well on the test. |
| | there. Finally, go for it! | Those who visualized the |
| | And come | process (i.e. studying) were more |
| | back to report how you | likely to study, which |
| | pulled it off | ultimately had the largest effect on |
| | and how it makes you feel. | their test performance. |
| | | If you own your own goals-and |
| | | then take steps towards |
| | | them-you're on your way to |
| | | increasing your overall well- |
| | | being. Studies show that when |
| | | people pursue goals intrinsic |
| | | to them, they're more motivated, |
| | | more likely to succeed, |
| | | and far happier than people who |
| | | don't have or don't pursue |
| | | strong dreams or aspirations. |
| A-05 | Routines are boring. Daily | Research shows that doing |
| | chores are | something tedious in a different |
| | boring. Life can be boring. | way can help people stay focused |
| | The key is | and more motivated. |
| | to power through things we | When we consider the purpose of |
| | don't | an activity, our attention |
| | want to do, because | shifts from the little mundane |
| | procrastination is | details to more abstract |
| | deadly-and an avoidable | concepts and principles. |
| | stressor. | Our beliefs about the activities we |
| | So take your least favorite | partake in can have a |
| | activities | significant effect on their |
| | and imagine you're living | outcomes. Researchers Ellen |
| | in some | Langer and Alia Crum split 84 |
| | pretend society that places | hotel workers into two |
| | enormous | groups, one a control and the other |
| | value on the people who | given information about |
| | perform that | how the work they do (cleaning |
| | activity. How do they | rooms) serves as good |
| | benefit from | physical fitness along with clear |
| | you engaging in this | examples. In just four |
| | activity? Now | weeks, the control group had no |
| | carry out that task with | measurable physical |
| | purpose and | changes, but the test group showed |
| | try to come up with | a number of changes |
| | reasons it's | including decreases in weight, |
| | important. | blood pressure, and body fat. |
| | Think of the guys who | Simply thinking about an activity |
| | repetitively | as being good for you or |
| | slice, dice, and roll sushi | good for others can help reinforce |
| | pieces for | its positive effects. |
| | hours on end. That can't be | |
| | that much | |
| | fun, right? And yet most | |
| | all of them | |
| | have a razor focus on their | |
| | work, | |
| | perhaps because they've | |
| | been trained | |
| | to believe they are creating | |
| | meaningful, artistic | |
| | products. | |
| A-06 | Think of a setback or | The most successful people are |
| | negative | those who can actively learn |
| | experience that occurred at | from their mistakes, handle |
| | least one | rejection and move forward. In |
| | year ago. How has that | other words, failure IS an option. |
| | event shaped | What matters is how you |
| | who you are today? Can | respond to it. |
| | you think of | Over the years, researchers have |
| | any benefits that came of | found that asking people to |
| | it? Spend | participate in expressive writing |
| | ten minutes writing about | about stressful experiences |
| | its impact | has beneficial psychological |
| | on your life. | effects, reducing stress and |
| | | producing long-term |
| | | improvements in mood. When |
| | | asked to |
| | | think about the good things that |
| | | came out of a bad |
| | | experience, the benefits were even |
| | | greater. People who |
| | | engage in “benefit-finding” |
| | | generally report less distress, |
| | | fewer disruptive thoughts, less |
| | | negativity, and more |
| | | meaningfulness in their lives. |
| | | Writing about extremely emotional |
| | | and personal topics also |
| | | has a positive health effect: One |
| | | study found that in |
| | | participants with elevated blood |
| | | pressure, it lowered blood |
| | | pressure for several months |
| | | following the exercise. |
| A-07 | If you brainstormed about | If you take the time to really think |
| | your core | about what's important to |
| | values in a previous | you, you'll be able to face life's |
| | activity, you may | challenges with increased |
| | have come up with things | confidence. |
| | like | Research shows that meaning in |
| | integrity, healthy living, or | life correlates positively to |
| | kindness. | mood stability and sociable |
| | If you haven't yet, take a | behavior, and meaning at work |
| | few | seems to inspire motivation and |
| | moments to write down | engagement. Going after |
| | your values. | goals that are important to us also |
| | (If you take the time to | has a slew of |
| | really think | psychological and physical |
| | about what's important to | benefits. |
| | you, you'll | Goals that include commitment to |
| | feel more committed to | something outside |
| | living them, | ourselves, such as our family, |
| | and be able to face life's | friends or community, |
| | challenges | promote life satisfaction. Zero-sum |
| | with increased | goals, such as a |
| | confidence.) | commitment to material gains or |
| | Now take what you learned | career success, actually |
| | and | can be detrimental to our |
| | brainstorm concrete ways | satisfaction with life. |
| | you can | Studies show that when we pursue |
| | make change your | goals that are meaningful |
| | everyday life to | or intrinsic to our values, we're |
| | better pursue the character | happier, more motivated, |
| | ideals you | and more likely to succeed. |
| | identified as being most | |
| | important. | |
| A-08 | Take an everyday activity | When we set meaningful goals, we |
| | you do | begin to direct our |
| | already, and transform it | energies in a way that aligns with |
| | into | what's important to us. |
| | something meaningful. So | And when we do something that |
| | if you're | we believe is meaningful, |
| | always tinkering with new | we feel good and achieve a sense |
| | recipes, | of purpose in our lives. |
| | compile your best ones | Studies have consistently found |
| | into a book | that the more meaning |
| | that you can pass along to | people report in their lives, the |
| | your | happier they are-and this is |
| | grandchildren one day. If | true of people in all life stages. |
| | you run | You don't necessarily have to help |
| | every morning, train a | others in order to make |
| | group of teens | your activity meaningful, but when |
| | to run a half-marathon. | you do, it amplifies the |
| | | effects of that activity and brings |
| | | about greater well-being. |
| | | Research shows that helping others |
| | | distracts us from our |
| | | own thoughts and concerns. |
| | | Anxiety and stress involve a |
| | | high degree of focus on self, and |
| | | focusing on the needs of |
| | | others helps us shift our thinking |
| | | and boosts our self- |
| | | esteem. |
| A-09 | Spend some money-it | Studies show that people who |
| | doesn't have | spend money on experiences |
| | to be a lot!-on a leisure | are happier than those who spend |
| | experience | money on material |
| | that will be meaningful and | possessions. Studies show this to |
| | rewarding | be true across most |
| | for you (concentrate on | demographic categories: male |
| | experiences, | orfemale, liberal or |
| | rather than material | conservative, high or low income |
| | things). | and religious preferences. |
| | For example, if you're | Life experiences become part of |
| | passionate | who we are. They're |
| | about music, take a music | woven into our memories and they |
| | class or go | shape our identity. In |
| | to a concert. If you're a | one study, researchers found that |
| | history buff, | spending money on |
| | you might visit a museum | positive experiences increased |
| | or historic | people's happiness. And the |
| | site near your town. | study showed that people who |
| | | spent money on a series of |
| | | smaller events were happier than |
| | | those who spent money on |
| | | one big event. |
| | | In other words, you'll be happier if |
| | | you spread out your |
| | | positive experiences rather than |
| | | spending money on “peak” |
| | | experience. |
| A-10 | Set a very long-term goal- | Professors Edwin Locke and Gary |
| | one that | Latham have studied |
| | can be completed in the | goal-setting methods for decades. |
| | span of | In numerous studies, |
| | several years. Maybe you | they've discovered the most |
| | finally | successful performance goals |
| | complete the memoir | (ones that can be measured, like |
| | you've wanted | getting a $10,000 salary |
| | to write for years. Or, you | raise or completing a half- |
| | go back to | marathon) are those that are both |
| | school in order to make a | challenging and specific, meaning |
| | major career | they can be clearly |
| | change. Imagine the | defined. |
| | benefits of | If we set the right goals-and then |
| | achieving that goal. What | take steps towards |
| | will | them-we get on the path to |
| | happen? How will you | increasing our overall well- |
| | feel? | being. Studies show that when |
| | | people pursue goals intrinsic |
| | | to them, they're more motivated, |
| | | more likely to succeed, |
| | | and far happier than people who |
| | | don't have or don't pursue |
| | | strong dreams or aspirations. |
| | | Intrinsic goals come from your |
| | | genuine values, beliefs, and |
| | | interests, rather than the |
| | | promise of fame or money, or the |
| | | wishes of others. |
| | | Focusing on the process, rather |
| | | than the outcome, can help |
| | | you achieve your goals. Research |
| | | by Pham and Taylor |
| | | found that students who visualized |
| | | themselves studying for |
| | | a test performed better than |
| | | students who visualized |
| | | themselves doing well on the test. |
| | | Those who visualized the |
| | | process (i.e. studying) were more |
| | | likely to study, which |
| | | ultimately had the largest effect on |
| | | their test performance. |
| | | When we set meaningful goals, we |
| | | begin to direct our |
| | | energies in a way that aligns with |
| | | what's important to us. |
| | | When we do something that we |
| | | believe is meaningful, we |
| | | achieve a sense of our own worth |
| | | and place in life. And |
| | | studies have consistently found |
| | | that the more meaning |
| | | people report in their lives, the |
| | | happier they are-and this is |
| | | true of people in all life stages. |
| A-11 | What upcoming events are | When we find ourselves thinking |
| | you | about our future, we're |
| | anticipating with joy and | usually worrying about something, |
| | optimism | increasing our levels of |
| | instead of anxiety? | anxiety. Even when we have |
| | Think of a future event, big | upcoming events we're truly |
| | or small, | excited about, most of us don't take |
| | that you're really excited | advantage of the |
| | about. | anticipatory experience. |
| | Imagine the details, | Research shows that anticipating |
| | including the | upcoming events prolongs |
| | sounds, smells, and tastes. | our excitement and boosts our |
| | Spend | optimism. Optimistic people |
| | some time putting yourself | are more likely to persevere when |
| | right in the middle of it. | the going gets tough. |
| | | They're good copers. They don't |
| | | give up. |
| | | When we have something to look |
| | | forward to we feel more |
| | | enthusiastic, motivated, and |
| | | psyched. And we're more |
| | | likely to behave in ways that |
| | | ensure our positive future |
| | | event will happen. |
| | | Studies show that the act of |
| | | anticipating future events also |
| | | lowers our levels of cortisol, the |
| | | stress hormone, because |
| | | when we believe good things are |
| | | coming our way, we're |
| | | happier in the day-to-day. |
| A-12 | What makes each of us | Gaining insight into what your |
| | special is that | strengths are-and then |
| | we all have unique | using more of those strengths in |
| | character | your daily life-can have a |
| | strengths-and when we use | major impact on your happiness. |
| | those | Christopher Peterson, |
| | strengths in novel ways, | Martin Seligman, and their |
| | we become | colleagues classified 24 |
| | happier. Once you've taken | signature character strengths (now |
| | the VIA | published as the Values |
| | survey to determine your | in Action (VIA) Classification), |
| | top five | including courage, wisdom, |
| | strengths, come up with a | curiosity, fairness, and self- |
| | way to use | regulation, that serve as the |
| | one of your strengths in a | essential building blocks of |
| | new and | positive character. |
| | different way this week. | Research has shown that using |
| | For example, | your top character strengths |
| | if one of your top | in novel ways can have long-term |
| | strengths is | effects on your well- |
| | appreciation of beauty and | being. In one study, people took a |
| | excellence, | survey to identify their |
| | go to a museum you've | character strengths and received |
| | never been to | feedback about their top |
| | before. If your strength is | five strengths. Then, they were |
| | kindness, | asked to use one of their top |
| | you could leave a big tip | strengths in a new and different |
| | on a small | way, every day for a week. |
| | check. If your strength | After one-month, three-month, and |
| | is courage, | six-month follow-ups, |
| | look for an opportunity to | people who continued to find new |
| | stand up for | ways to exercise their |
| | someone else who needs | strengths saw the biggest increases |
| | support. You | in happiness and the |
| | get the idea! | biggest decreases in depression. |
| | | (Merely identifying |
| | | signature strengths can lead to a |
| | | quick mood boost, but it |
| | | doesn't have the long-term effects |
| | | of actually using your |
| | | strengths.) |
| | | Using signature strengths at work |
| | | has also been linked to |
| | | increased happiness and job |
| | | satisfaction, and strengths- |
| | | based school interventions have |
| | | shown to improve student's |
| | | social skills, engagement in school, |
| | | and “learning” strengths |
| | | (like greater curiosity and an |
| | | increased love of learning). |
| E-01 | As you go through your | Self-compassion boosts our |
| | day, pay | resilience so that when we |
| | attention to your inner | stumble, we can get up and try |
| | voice-and | again. When making a |
| | when you catch your inner | mistake isn't a big deal, we're more |
| | voice | open to learn from our |
| | berating you for something | missteps and try new things, rather |
| | or other- | than hide in shame. |
| | catch yourself. | Kristin Neff, Ph.D., an associate |
| | Come up with with a | professor at the University |
| | phrase or mantra | of Texas at Austin, has been |
| | to shift your thinking. | researching self-compassion |
| | “Live and | for well over a decade. Neff |
| | Learn” or “This too shall | believes today's competitive |
| | pass”-or | culture leads us to believe being |
| | whatever works for you. | good enough isn't good |
| | Then, focus | enough. Media images and slogans |
| | on what makes you great. | push us to compare |
| | What have | ourselves with others, even though |
| | you overcome or | there will always, |
| | achieved? You can | always be someone who is more |
| | even create a narrative of | successful, richer, skinnier, |
| | your life | or more attractive than we are. |
| | thus far, highlighting the | Neff s research shows that self- |
| | aspects that | compassionate people feel |
| | showcase your positive | more secure and strongly suggests |
| | traits. | that accepting our |
| | | imperfections may be the first step |
| | | toward better health. |
| | | People who are easier on |
| | | themselves are less likely to be |
| | | depressed and anxious and are |
| | | more prone to happiness and |
| | | optimism. Self-compassion steps |
| | | in precisely when we fall |
| | | down, allowing us to get up and try |
| | | again. |
| E-02 | Pick a person and spend | Research clearly suggests that |
| | some time | when we empathize with |
| | imagining what it'd be like | others and try to see the world |
| | to be | through other's eyes, we're |
| | them. What is life like for | more likely to treat them with |
| | them? What | kindness. And when we |
| | must they be feeling and | appreciate someone else's point of |
| | why? | view, he/she will most |
| | Write down some thoughts | likely become more sympathetic |
| | about your experience. | and understanding of ours. |
| | | In studies, scientists have found |
| | | that people are generally |
| | | kinder and more helpful after |
| | | taking others' perspectives, as |
| | | it increases compassionate |
| | | emotions towards the other |
| | | person. This exercise also leads us |
| | | to view and treat others |
| | | more like ourselves and attribute |
| | | more positive traits to |
| | | others. |
| | | That alone is a pretty good reason |
| | | to start exercising our |
| | | compassion muscles. |
| E-03 | Think of someone you've | Most of us spend time dwelling on |
| | positively | our negative traits and |
| | impacted, whether it's | often neglect to recognize our |
| | someone from | virtues. One study showed |
| | your inner circle or a | that reframing negative thoughts |
| | complete stranger. | and boosting self-esteem |
| | Now imagine what it's like | through someone else's perspective |
| | for him or | is a very effective way |
| | her to be on the receiving | to increase resilience, |
| | end of your kindness. | resourcefulness, optimism and |
| | What positive impact have | positive thinking. |
| | you had on | Research shows that self- |
| | his or her life? Are there | compassion comes with mental |
| | ways you | health benefits like less depression, |
| | could be even more | more optimism, greater |
| | helpful? | happiness, more life satisfaction. |
| | | Scientists have also found |
| | | that people who respond to life's |
| | | challenges with self- |
| | | compassion are happier, healthier, |
| | | more proactive, and more |
| | | conscientious than those who are |
| | | not. People who are kind |
| | | to themselves seem to take better |
| | | care of themselves AND |
| | | others. Self-compassionate people |
| | | are more resilient, feel |
| | | more connected to others, and are |
| | | less depressed and |
| | | anxious than others. |
| | | So when you find yourself feeling |
| | | guilty for not doing |
| | | enough, consider the positive |
| | | impact you have on the people |
| | | around you and have compassion |
| | | for YOURSELF. |
| E-04 | You're right, they're | Research clearly suggests that |
| | wrong. Okay, | when we empathize with |
| | fine, sure, whatever. But | others and try to see the world |
| | that attitude | through other's eyes, we're |
| | isn't going to get you far | more likely to treat them with |
| | when it | kindness. And when we |
| | comes to patching things | appreciate someone else's point of |
| | up. Time to | view, he/she will most |
| | try a different approach. | likely become more sympathetic |
| | When someone close to | and understanding of ours. |
| | you does or | Studies have found that people are |
| | says something that you | generally kinder and |
| | don't | more helpful after perspective- |
| | understand, try looking at | taking, as it increases |
| | the situation | compassionate emotions towards |
| | a little differently. | the other person. |
| | What led up to it? What | This exercise also leads us to view |
| | context might | and treat others more |
| | better explain his/her | like ourselves. Scientists found that |
| | behavior? We | when we actively try to |
| | all do strange things once | understand another person's |
| | in awhile, | perspective, we think of them |
| | especially when we're | more positively afterwards. |
| | tired, hungry, | |
| | angry or lonely. | |
| E-05 | Think of someone you | Research has found that |
| | cross paths | connecting with people different |
| | with but rarely speak to- | from us broadens our compassion |
| | someone | and awareness and |
| | who is different than you. | deepens our sense of community |
| | Plan to | while opening us up to |
| | strike up a conversation | new perspectives on life. When we |
| | with this | try to take the |
| | person the next time you | perspective of others, we behave |
| | see him/her. | more admirably and |
| | Talk to the elderly lady at | generously, as it increases |
| | the supermarket. Share | compassionate emotions. |
| | gardening tips | Perspective-taking also makes us |
| | with a neighbor. Offer | view-and treat-others |
| | lunch to the kid | more like ourselves. |
| | who mows your lawn. | According to researchers at the |
| | Make a plan to make a | University of California- |
| | connection | Berkeley, young adults from |
| | with them in your spare | affluent backgrounds are less |
| | time, then | empathetic to the suffering of |
| | give it a try and report | others than people whose |
| | back. | upbringing involved some |
| | | financial struggle. It's not that |
| | | they're cold-hearted-scientists |
| | | believe they simply aren't |
| | | as adept as recognizing the signals |
| | | of suffering because they |
| | | haven't experienced those types of |
| | | obstacles in their lives. |
| E-06 | Think of someone you | Research clearly suggests that |
| | know-perhaps | when we empathize with |
| | a friend or coworker-who | others and try to see the world |
| | disagrees | through other's eyes, we're |
| | with you about something | more likely to treat them with |
| | rather | kindness. And when we |
| | minor. Maybe they don't | appreciate someone else's point of |
| | understand | view, he/she will most |
| | the genius of your favorite | likely become more sympathetic |
| | TV show, | and understanding of ours. |
| | or perhaps they can't stand | Studies have found that people are |
| | cats, and | generally kinder and |
| | you've got five of them | more helpful after perspective- |
| | slinking | taking, as it increases |
| | around your home. | compassionate emotions towards |
| | Take a moment to think | the person. |
| | about what | This exercise also leads us to view |
| | it's like to be them. Why | and treat others more |
| | do they feel | like ourselves. Scientists found that |
| | differently from you? | when we actively try to |
| | | understand another person's |
| | | perspective, we think of them |
| | | more positively afterwards. |
| E-07 | The next time someone | This activity asks you to consider a |
| | close to you | mindset that differs |
| | acts in a way that hints or | from your default one, which is |
| | upsets you, | what perspective-taking is |
| | take a few moments to | all about. Researchers have found |
| | think about the | that people are generally |
| | context and try to | kinder and more helpful after |
| | understand what | perspective-taking, as it |
| | might have led to their | increases compassionate emotions |
| | behavior. | towards the other person. |
| | What circumstances or | This exercise also leads us to view |
| | events might | and treat others more |
| | have led them to act the | like ourselves. Scientists found that |
| | way they did? | when we actively try to |
| | Were they really trying to | understand another person's |
| | offend you? | perspective, we think of them |
| | | more positively afterwards. |
| | | Keep in mind: It's not too much of |
| | | a stretch to empathize |
| | | with someone when they act in a |
| | | strange or perplexing way, |
| | | but the exercise becomes more |
| | | challenging when your |
| | | feelings (or your ego!) are |
| | | involved. And it'll get even more |
| | | difficult to empathize with |
| | | someone when you're engaged |
| | | in an outright conflict with them. |
| | | (Hey, we never said |
| | | empathy was easy to master!) |
| | | In a situation where your feelings |
| | | are hurt, you may find |
| | | that you feel quite differently after |
| | | you take a step back and |
| | | think about the context. |
| E-08 | Think of what it's like to | When it comes to empathy and |
| | be someone | understanding where others |
| | with whom you disagree | are coming from, it might not be |
| | strongly | too difficult a task when |
| | about a topic that's | you merely have different opinions |
| | moderately | about a fairly neutral |
| | important to you. Can you | topic, like where to find the best |
| | come up | cupcakes in town or the |
| | with reasons why they | best singer on “The Voice”, but it's |
| | might think or | not so easy when you |
| | feel differently? | disagree about something that's |
| | For example, maybe you're | pretty important to you. |
| | a die-hard | That's where perspective-taking |
| | Giants fan in the middle of | comes in. Studies have |
| | a bar filled | found that people are generally |
| | with Pats fans. Why are | kinder and more helpful |
| | they such | after this exercise, as it increases |
| | staunch supporters? Or, | compassionate emotions |
| | think about | towards the other person. |
| | your coworker who's | This exercise also leads us to view |
| | been a | and treat others more |
| | vegetarian for 10 years- | like ourselves. Scientists found that |
| | maybe she | when we actively try to |
| | doesn't believe in eating | understand another person's |
| | meat because | perspective, we think of them |
| | she had an eye-opening | more positively afterwards. So |
| | experience at a farm. | why is this important? Being |
| | | able to take another person's point |
| | | of view is crucial to |
| | | making and keeping friends. |
| | | Empathy fosters deep social |
| | | connections, and the benefits even |
| | | extend to our romantic |
| | | lives: In a study of couples, people |
| | | who scored higher in |
| | | perspective-taking were happier in |
| | | their relationships. |
| E-09 | Reflect on a recent conflict | This activity asks you to consider a |
| | you had | mindset that differs |
| | with someone that really | from your own, which is what |
| | hurt your | perspective-taking is all |
| | feelings. For example, | about. You've probably tried |
| | maybe your | empathizing with a friend or |
| | spouse insulted | coworker who's behaved strangely |
| | you during an | or even in a hurtful |
| | argument about the | manner-but now you're ready to |
| | family's spending | practice empathy during |
| | habits, or your | a conflict or argument, which, we |
| | sister made a cutting | admit, can be quite a |
| | remark during your last | challenge for most folks! |
| | get-together. | In a situation where your emotions |
| | Try to gain some insight | are stirred up and your |
| | into where | feelings are hurt, you may find that |
| | they were coming from. | you feel quite |
| | Why were | differently after you take a step |
| | they at odds with you? | back, mentally remove |
| | What was their | yourself from the situation, and |
| | perspective? | think about the context. |
| | | Researchers have found that |
| | | people are generally kinder and |
| | | more helpful after perspective- |
| | | taking, as it increases |
| | | compassionate emotions towards |
| | | the other person. This |
| | | exercise also leads us to view and |
| | | treat others more like |
| | | ourselves. Scientists found that |
| | | when we actively try to |
| | | understand another person's |
| | | perspective, we think of them |
| | | more positively afterwards. |
| E-10 | Think of something you | Research shows that helping others |
| | can do in | distracts us from our |
| | your spare time that will | own thoughts and concerns. |
| | allow you to | Anxiety and stress involve a |
| | improve the life of | high degree of focus on self, so |
| | someone very | focusing on the needs of |
| | different from yourself, | others helps us shift our thinking |
| | whether it's | and boosts our self- |
| | tutoring a child or | esteem. In a 2015 study, |
| | spending time with | researchers found that small acts of |
| | an elderly resident at a | kindness like opening a door or |
| | nursing home. | simply asking someone if |
| | Then, do it! | they needed help buffered people |
| | | against everyday stress |
| | | and made them feel happier. |
| | | Once you've started helping |
| | | someone, don't be surprised if |
| | | it feels pretty, well, natural. In |
| | | studies of human |
| | | cooperation, David Rand of |
| | | Harvard found that the first |
| | | impulse of adults is to help others, |
| | | and the same instinct to |
| | | help others achieve goals has been |
| | | found in infants (and |
| | | even chimpanzees and rats). |
| | | A multi-year study from |
| | | researchers at three universities |
| | | also found that helping others may |
| | | not only help buffer the |
| | | negative effects of stress, but |
| | | lengthen our lives. Lastly, acts |
| | | of kindness are contagious. |
| | | Research shows that when |
| | | people benefit from kindness they |
| | | “pay it forward” by |
| | | helping others, creating a spiral |
| | | effect for more and more |
| | | acts of kindness. |
| E-11 | Imagine what it'd be like | When you imagine stepping |
| | for one of | outside of yourself and into the |
| | your close friends, your | shoes of someone close to you, you |
| | spouse, or | have the opportunity to |
| | one of your family | experience an entirely new |
| | members if you | perspective on life-and your |
| | disappeared, or had never | role in it. Scientists have found that |
| | existed in | when people participate |
| | the first place. In what | in a perspective-taking exercise |
| | ways would | like this one, they tend to |
| | their lives be worse? What | act with more kindness and |
| | would they | helpfulness, as it increases |
| | miss the most about you? | compassionate emotions towards |
| | | the person whose |
| | | perspective has been taken. |
| | | This exercise also leads us to view |
| | | and treat others more |
| | | like ourselves. Scientists found that |
| | | when we actively try to |
| | | understand another person's |
| | | perspective, we think of them |
| | | more positively afterwards. So |
| | | why is this important? Being |
| | | able to take another person's point |
| | | of view is crucial to |
| | | making and keeping friends. |
| | | Empathy fosters deep social |
| | | connections, and the benefits even |
| | | extend to our romantic |
| | | lives: In a study of couples, people |
| | | who scored higher in |
| | | perspective-taking were happier in |
| | | their relationships. |
| E-12 | Think of what it's like to | At this point, you're practically a |
| | be someone | pro at walking in other |
| | (real or hypothetical) with | people's shoes. You'll probably |
| | whom you | agree that it's not too |
| | vehemently disagree about | difficult a feat when you simply |
| | something | think differently about a |
| | very important to you-for | fairly neutral topic, but |
| | example, | empathizing with |
| | over a hot-button political | someone who has |
| | or religious issue. | opinions you violently disagree |
| | What challenges do they | with can seem practically |
| | face? Why | impossible. However, we know |
| | do they feel as they do? | you can do it with a little |
| | We know it's | effort in perspective-taking! |
| | not easy, but in your mind, | Studies have found that people feel |
| | try to treat | more compassionate |
| | this person with sympathy, | emotions and behave more |
| | understanding, and | admirably after this exercise, as |
| | acceptance. | it increases compassionate |
| | | emotions towards the other |
| | | person. |
| | | This exercise also leads us to view |
| | | and treat others more |
| | | like ourselves. Scientists found |
| | | that when we actively try to |
| | | understand another person's |
| | | perspective, we think of them |
| | | more positively afterwards. So |
| | | why is this important? Being |
| | | able to take another person's point |
| | | of view is crucial to |
| | | making and keeping friends. |
| | | Empathy fosters deep social |
| | | connections, and the benefits even |
| | | extend to our romantic |
| | | lives: In a study of couples, people |
| | | who scored higher in |
| | | perspective-taking were happier in |
| | | their relationships. |
| E-13 | Creating the intention to | Experiences of positive emotions |
| | seek out and | do more than feel good. |
| | create micro-moments of | Research shows they also “do |
| | loving | good” in that they can |
| | connection can be a tool | broaden your outlook and build |
| | for elevating | your resilience and |
| | your well-being. Today, | resourcefulness-even your physical |
| | aim for three | health. |
| | micro-moments filled with | New evidence suggests that this |
| | warmth, | may be especially true for |
| | respect and goodwill with | the positive emotions that you |
| | a family | share with others in real- |
| | member, friend, colleague, | time, face-to-face interactions. |
| | or even | Micro-moments of positive |
| | someone you don't know | connection allow positive emotions |
| | that well. | to reverberate between |
| | Freely offer your attention | people, creating a powerful |
| | and eye | resonance of good feeling and |
| | contact as you talk or | goodwill. |
| | touch. Later, | Barbara Fredrickson, Ph.D., Kenan |
| | lightly reflect on whether | Distinguished Professor |
| | these | of Psychology and Nemoscience at |
| | exchanges brought you a | the University of North |
| | feeling of | Carolina at Chapel Hill, has been |
| | positivity resonance: a | investigating the various |
| | back-and-forth | benefits of positive emotions for |
| | reverberation of positive | 25 years and her latest |
| | energy. | book, Love 2.0, spotlights the |
| | | benefits of what she calls |
| | | “positivity resonance.” |
| | | Experimental evidence from |
| | | Fredrickson and her team suggests |
| | | that positivity resonance |
| | | may be the active ingredient that |
| | | links good feelings to good |
| | | health. |
| G-01 | Buy a little something for | Acts of kindness are contagious. |
| | anyone you | Research shows that when |
| | want and surprise him/her | people benefit from kindness they |
| | with it. | “pay it forward” by |
| | Ideally, it would be | helping others, creating a spiral |
| | something that | effect for more and more |
| | will spark some interaction | acts of kindness. |
| | with them | But when you give to others, no |
| | (if you buy someone a | one benefits as much as |
| | book, you can | you. One study gave participants a |
| | discuss it together; if you | sum of money and then |
| | buy | asked some of them to spend the |
| | someone a cup of coffee, | money on themselves. The |
| | you can chat | others were told to spend it on |
| | while drinking.) | someone else. The people |
| | | who spent money on someone else |
| | | were significantly |
| | | happier than those who spent the |
| | | money on themselves. |
| G-02 | Most of us do nice things | Have you ever experienced a |
| | for other | “helper's high” while assisting |
| | people without really | a neighbor, volunteering, or |
| | thinking about | donating goods to others? Or |
| | it. Today you're | maybe while lending an ear to a |
| | going to do | friend, or passing on your |
| | something nice for 5 | skills to someone else? |
| | different people. | Dr. Sonja Lyubomirsky, |
| | Your “act of kindness” | psychology professor at University |
| | can be | of California, Riverside, asked |
| | anything-call your mom, | students to commit five |
| | hold the | random acts of kindness a week for |
| | door for a stranger, | six weeks. One group |
| | compliment a co- | had to perform five kind acts in a |
| | worker-or use your | single day for each of |
| | imagination and | those weeks, whereas the other |
| | stretch yourself. Buy | group could spread their |
| | groceries for an | kind acts out over the course of the |
| | elderly neighbor or help a | week for six weeks. |
| | co-worker | Guess which group showed a 42% |
| | with a project. Hold a door | increase in happiness? |
| | open for | The students who had to perform |
| | someone or add coins to an | five acts of kindness in |
| | expiring parking meter. | one day. |
| | You decide! You might | That's why doing five acts of |
| | be surprised | kindness in one day is so |
| | by how much it | effective. In order to get it done, |
| | cheers you up. | we have to plan ahead, |
| | | refocus our priorities and shift our |
| | | thinking. Studies also |
| | | show that regularly performing |
| | | kind acts makes people |
| | | happy for extended periods of |
| | | time, but that mixing up the |
| | | types of kind acts is key. To |
| | | maintain the happiness |
| | | boosting effects of the act, people |
| | | had to vary the types of |
| | | acts they did; otherwise it became |
| | | a tedious experience. All |
| | | the more reason to be creative! On |
| | | top of it all, behavior that |
| | | distracts us from our own problems |
| | | also boosts our self- |
| | | esteem and gives us a sense of |
| | | purpose. |
| G-03 | What nice thing did | Research shows helping others |
| | you do (or are | distracts us from our |
| | you planning to do) for | thoughts and concerns. Anxiety |
| | someone today? | and stress involve a high |
| | Whether it's running an | degree of focus on self, so |
| | errand for a | attending to the needs of others |
| | busy friend, baking | helps us shift our thinking and |
| | cookies for a | boosts our self-esteem. In a |
| | neighbor or mentoring a | 2015 study, researchers found that |
| | child, record it here. | small acts of kindness |
| | You can repeat (and | like opening a door or simply |
| | report!) this | asking someone if they |
| | activity as often | needed help buffered people |
| | as you'd like. | against everyday stress and |
| | | made them feel happier, and a |
| | | 2016 study found that being |
| | | kind to others gives us a bigger |
| | | wellbeing boost than being |
| | | kind to ourselves. |
| | | Studies also show that regularly |
| | | performing kind acts makes |
| | | people happy for extended periods |
| | | of time, but that mixing |
| | | up the types of kind acts is key. To |
| | | maintain the happiness |
| | | boosting effects of the act, people |
| | | had to vary the types of |
| | | acts they did; otherwise it became |
| | | a tedious experience. All |
| | | the more reason to be creative! |
| G-05 | Spend an hour devoting | According to research, we find the |
| | yourself to an | most gratification from |
| | activity that you find | experiences we've chosen for |
| | meaningful. You | ourselves that fully engage |
| | could select a skill that's | and absorb us, that satisfy and |
| | valuable to | fulfill us and that allow us to |
| | you and invest an hour | connect us with others. Our minds |
| | developing it | want to be fully engaged |
| | (for example, getting better | in activities that are meaningful. |
| | at playing | Learning new things, |
| | the guitar) or try | pursuing hobbies, being creative |
| | something new. | with our leisure time- |
| | (Make a plan to start a new | these are the activities of a |
| | hobby or | seeking mindset. |
| | take a class, or listen to | One recent study by Chancellor |
| | live music or | and Lyubomirsky suggested |
| | read a new book.) | “the most satisfying pursuits |
| | Just plan it and do it. | should involve learning new |
| | | skills (e.g. mastering a new |
| | | instrument or learning a foreign |
| | | language), spending time with |
| | | others (e.g. taking out one's |
| | | family to dinner or having coffee |
| | | with a friend), or doing |
| | | something good for someone else |
| | | (e.g. buying holiday |
| | | decorations for an elderly neighbor |
| | | or sending a care |
| | | package to a sick friend).” |
| G-06 | Your choice. Come up | Life experiences become part of |
| | with an | who we are. They're |
| | experience you find | woven into our memories and they |
| | meaningful and | shape our identity. |
| | ask someone to share it | Research shows that people who |
| | with you-on | spend money on |
| | your dime. (Remember, it | experiences are happier than those |
| | doesn't | who spend money on |
| | have to be expensive to be | material possessions. And it's true |
| | worthwhile!) | across most demographic |
| | | categories: male or female, liberal |
| | | or conservative, high or |
| | | low income and religious |
| | | preferences. The study also |
| | | showed that people who spent |
| | | money on a series of smaller |
| | | events were happier than those |
| | | who spent money on one big |
| | | event. In other words, you'll be |
| | | happier if you spread out |
| | | your positive experiences rather |
| | | than spending money on |
| | | “peak” experience. |
| | | When you share the experience |
| | | with others, you get even |
| | | more bang for your buck. Shared |
| | | experiences strengthen |
| | | bonds between friends, couples, |
| | | and families. And |
| | | according to scientists, the one |
| | | thing that sets apart the |
| | | happiest 10 percent of the |
| | | population from everyone else is |
| | | (drumroll) . . . the strength of their |
| | | social bonds. Yale |
| | | researchers also found that when |
| | | people pay attention to the |
| | | same pleasant thing, whether it's a |
| | | song, a dessert, or a |
| | | scenic view, the experience is |
| | | much more pleasurable. In |
| | | other words, we get more joy out |
| | | of experiences when |
| | | they're shared than we do alone. |
| G-07 | When something good | Most of us already share the big |
| | happens to | milestones in our lives with |
| | someone close to you, | others-engagements, births, |
| | make a point of | promotions, marriage. |
| | helping him or her | Research suggests that the sharing |
| | prolong the | of good fortune actually |
| | excitement. Tell them how | contributes to our well-being, and |
| | much they | that the benefits of |
| | deserve it, ask lots of | sharing our good fortune can |
| | questions, | extend beyond the happiness |
| | encourage them to | from the event itself! |
| | commemorate the | Perhaps less well known is the |
| | occasion-or even plan | power of “capitalization”: |
| | something to | When we show enthusiasm for |
| | help them celebrate with | others' goals and the |
| | you and others! | milestones they are hitting along |
| | | the way, we are |
| | | capitalizing, or prolonging our |
| | | positive feelings about |
| | | something important to us. Studies |
| | | show that amplifying the |
| | | pleasure of a good situation that |
| | | involves others contributes |
| | | to an upward spiral effect of |
| | | positive emotion that boosts |
| | | happiness. |
| | | Shelly Gable's research also shows |
| | | that when we respond |
| | | to, and share, other people's good |
| | | news, they feel more |
| | | understood, validated and cared |
| | | for. This type of sharing |
| | | enhances relationship quality, and |
| | | increases happiness and |
| | | life satisfaction. |
| G-08 | Identify something that | Forgiving someone for a slight or |
| | someone close | an annoyance is often |
| | to you does that often | easier than it sounds. However, |
| | annoys or | people have an easier time |
| | upsets you. See if you can | forgiving those whom they feel |
| | become | close to, like friends and |
| | more forgiving about that | family, or people for whom they |
| | particular | feel empathy. |
| | behavior. Can you be more | As difficult as forgiveness can be, |
| | understanding about why | the overwhelming |
| | they do it? | benefits are worth it from both a |
| | Can you be more patient | physical and emotional |
| | with them | standpoint: |
| | when it occurs? | Researchers have found that |
| | | people who tend to forgive are |
| | | less anxious, less depressed and |
| | | less hostile. They also have |
| | | higher levels of well-being, |
| | | positive emotion, and are more |
| | | satisfied with their lives in general. |
| | | In studies measuring |
| | | cardiovascular reactivity, people |
| | | who imagined forgiving a |
| | | transgression they've experienced |
| | | had lower blood pressure |
| | | and heart rate than participants |
| | | asked to hold a grudge. |
| | | Finally, those who readily forgive |
| | | have a reduced risk for |
| | | nicotine dependence and substance |
| | | abuse. |
| | | Isn't that enough to convince you |
| | | to be more forgiving? We |
| | | think so. |
| G-09 | Plan an activity or | One of the most precious things |
| | excursion with one | you can give someone is |
| | or more members of your | your own time. You'll experience a |
| | inner | bigger mood boost after |
| | circle-perhaps a group of | doing this activity if you take your |
| | your best | time without employing |
| | friends, or your partner or | any shortcuts. So when you're |
| | child. It | planning your activity, select |
| | doesn't need to be | a day when you're not feeling |
| | expensive-in fact, | rushed or guilty about leaving |
| | it might not cost any | other duties undone. |
| | money at all-but | In 2002, researchers found that |
| | make sure the activity | people who spent less time |
| | emphasizes | alone and more time talking to |
| | new experiences that will | others tended to be happier. |
| | involve a lot | Ten years later, a different team |
| | of interaction with | showed how to maximize |
| | each other. | the feel-good power of |
| | | conversation: Spend less time on |
| | | chitchat. The happiest people, it |
| | | turns out, have a third as |
| | | much small talk and twice as many |
| | | meaningful |
| | | conversations as the least happy |
| | | people. Yale researchers |
| | | also found that when people pay |
| | | attention to the same |
| | | pleasant thing, whether it's a song, |
| | | a dessert, or a scenic |
| | | view, the experience is much more |
| | | pleasurable. In other |
| | | words, we get more joy out of |
| | | experiences when they're |
| | | shared than we do alone. |
| G-10 | Think of a grudge you're | We feel generous when we forgive |
| | still holding | someone for a slight or |
| | against someone for | an annoyance, but it's more |
| | something they | difficult to forgive someone |
| | did that offended you. It | who's truly offended us. As hard as |
| | may not be | it is, the overwhelming |
| | easy, but perform a | benefits are worth it from both a |
| | forgiveness | physical and emotional |
| | exercise to see if you can | standpoint: |
| | let go of | Researchers have found that |
| | your hard feelings. | people who tend to forgive are |
| | Think of the energy that | less anxious, less depressed and |
| | goes into | less hostile. They also have |
| | holding on to this grudge, | higher levels of well-being, |
| | and assess | positive emotion, and are more |
| | whether there are others in | satisfied with their lives in general. |
| | your life | In studies measuring |
| | that are being affected | cardiovascular reactivity, people |
| | by your | who imagined forgiving a |
| | inability to forgive this | transgression they've experienced |
| | person. | had lower blood pressure |
| | Perhaps you can even | and heart rate than participants |
| | think of a few | asked to hold a grudge. |
| | benefits that arose as a | Finally, those who readily forgive |
| | result of the offense. | have a reduced risk for |
| | | nicotine dependence and |
| | | substance abuse. |
| | | To help facilitate forgiveness, it's |
| | | useful to look for ways a |
| | | personal transgression may have |
| | | benefited you in some |
| | | way. In a writing intervention by |
| | | McCullough, Root, and |
| | | Cohen, people who spent 20 |
| | | minutes writing about the |
| | | benefits that arose from a |
| | | transgression reported more |
| | | forgiveness than people in a |
| | | control group who were asked |
| | | to write about an unrelated topic or |
| | | an unpleasant part of the |
| | | transgression. |
| G-11 | Identify a cause that you | Volunteering and helping others |
| | care about | distracts us from our own |
| | and volunteer your time to | thoughts and concerns. Depression, |
| | furthering | anxiety and stress |
| | it in some tangible way. | involve a high degree of focus on |
| | If education | ourselves, so when we |
| | is important to you, | have a positive impact on someone |
| | consider donating | else, we boost our mood |
| | tutoring students or | and self-esteem, and give our life a |
| | helping high | greater sense of purpose. |
| | school students with | A study conducted by the |
| | their college | University of British Columbia |
| | admission essays. If you're | and Harvard Business School |
| | an animal lover, spend | suggests that kindness and |
| | Saturday mornings | happiness form what's called a |
| | walking dogs from the | positive feedback loop. |
| | local shelter. | When we do something nice, we |
| | | feel happier. And the |
| | | happier we feel, the more likely |
| | | we'll perform another kind act. |
| | | Volunteering is also good for your |
| | | health: Researchers have |
| | | found that helping others is |
| | | associated with reduced stress |
| | | and a longer life! |
| G-12 | What cause is important to | A study conducted by the |
| | you? Make | University of British Columbia |
| | a charitable contribution | and Harvard Business School |
| | (you don't | suggests that giving and |
| | have to break the bank!) | happiness form what's called a |
| | that will | positive feedback loop. |
| | allow you to directly | Giving to charity makes us |
| | witness a person | happier, and when we're |
| | (or group of people) | happier, we give more. And |
| | benefiting from | believe it or not, they also |
| | your generosity. For | found that people actually feel |
| | example, maybe | wealthier when they give |
| | your donation to a local | money away! |
| | community | Similarly, researchers gave |
| | garden allows | participants a sum of money and |
| | everyone in the | then asked some of them to spend |
| | neighborhood to have easy | the money on themselves. |
| | access to | The others were told to spend it on |
| | fresh vegetables. | someone else. The |
| | Or, you chip in for | people who spent money on |
| | an operation that a | someone else were |
| | friend's son | significantly happier than those |
| | desperately needs | who spent the money on |
| | but can't afford. | themselves. |
| | | When we can see the reactions of |
| | | those we've helped, the |
| | | effect is even more powerful. |
| | | In a national survey of 3,300 |
| | | participants, volunteers who |
| | | witnessed the reactions of the |
| | | people they helped reported |
| | | increased self-esteem, less |
| | | stress, and a greater sense of the |
| | | “helper's high.” |
| | | And neuroscience backs this up: In |
| | | a brain imaging study |
| | | conducted at the Washington |
| | | University School of |
| | | Medicine, scientists found that the |
| | | reward centers of the |
| | | brain lit up when participants |
| | | anonymously made a |
| | | charitable donation-these are the |
| | | same parts of the brain |
| | | that light up when people |
| | | experience pleasure, like money, |
| | | sex, or a really decadent ice cream |
| | | sundae. |
| R-01 | Good morning, sunshine! | One of the systems in the brain that |
| | Time to | controls when we sleep |
| | look at the bright side. | and when we're awake is called the |
| | Literally. This | internal circadian clock. |
| | week, aim to wake up at | This clock is highly sensitive to |
| | the same | light, and research shows |
| | time each morning | that bright light in the morning can |
| | (resist the urge to | help wake you up and |
| | snooze the alarm!), | have a positive effect on your |
| | and get some | energy levels and hormone |
| | sunlight as soon as you | production. Exposing yourself to |
| | can. Open | light early in the morning |
| | your curtains and let the | can also help improve your sleep at |
| | light fill your | night by helping your |
| | room, go for a walk, or | circadian clock know when to start |
| | simply sit | winding down. |
| | outside in the sun with | The benefit of purposely exposing |
| | your favorite | yourself to light is |
| | wake-up beverage. | supported by a whole lot of |
| | What's the bright | science. Research shows that a |
| | idea behind this? | regular schedule is good for your |
| | Exposure to light | circadian rhythm, which is |
| | when you first wake up | what wakes you up at the right |
| | will help reset | time each day, as long as |
| | your circadian clock | you keep your routine consistent. |
| | and improve | If you go to bed and wake |
| | your energy levels during | up at a different time each day, the |
| | the day. It | circadian rhythm will get |
| | will also help your body | confused and you'll be awake or |
| | get the | sleepy at all the wrong times. |
| | message that nighttime | The other benefit of a regular |
| | is for sleeping. | schedule is that it helps your |
| | (Option:) Create a positive | brain know what to expect and to |
| | wake-up | make sure you sleep |
| | routine that includes | deeply. Your brain also anticipates |
| | activities you | when you want to wake |
| | love. For example, you | up and releases chemicals, |
| | could play | including cortisol, to help you |
| | upbeat music, use | wake up and feel awake. If you |
| | a shower product | wake up too early or too |
| | with a scent you find | late, you won't get this benefit. |
| | energizing, drink | Having a fresh and happy morning |
| | really good coffee, or | routine can also tell your |
| | take a moment | body when it's time to feel awake |
| | to think of what you're | and energized. If you |
| | grateful for | wake up excited and ready to go, |
| | and what you want to | you perform better |
| | accomplish that | throughout the day and feel less |
| | day. Before long, you | disoriented when you wake up. |
| | may find | |
| | yourself excited to | |
| | wake up every | |
| | morning because you get | |
| | to do all | |
| | these awesome things! | |
| R-02 | Autogenic training is a | Autogenic training is similar to |
| | relaxation | meditation in that it helps |
| | technique that can | you feel calm. And calming your |
| | help restore | mind to pay attention to |
| | balance to mind and body. | the sensations and rhythms in your |
| | It offers a | body has a ton of |
| | wide range of benefits, | benefits. It can help you to |
| | from reducing | function better and feel less |
| | insomnia to improving | anxious during the day and also |
| | sleep patterns. | wind down more easily and |
| | This guided practice is | sleep better at night. This |
| | designed to | relaxation technique typically |
| | support you to fall asleep | involves a series of statements |
| | faster and | about the heaviness or |
| | wake up feeling more | warmth of various places in the |
| | energized. Find | body. Over time, you may |
| | a quiet place free of | find that it takes less and less time |
| | distractions and | to bring about a peaceful |
| | try this brief guided | state of mind. |
| | autogenic training | |
| | exercise to help you ease | |
| | into a rest | |
| | and relaxation mindset. | |
| R-03 | Step away from your work | Consistently getting into bed |
| | or must- | feeling relaxed and prepared |
| | dos one horn before your | for sleep can have a profound |
| | bedtime | impact on sleep health in both |
| | routine, and turn off your | the short and long term. |
| | electronic | Having a predictable, pleasant |
| | devices at least 30 minutes | bedtime routine lets you feel |
| | before | your natural sleep pressure. If you |
| | bedtime. Use this time to | work out, watch an |
| | sit with | intense movie, or have a lot of |
| | yourself and think | social interaction before bed, |
| | through your day. | however, chances are you're going |
| | What were the highlights? | to feel wired and not at |
| | Did you | all ready for sleep. Give yourself |
| | inch closer toward a goal | space between your daily |
| | or follow | activities and bedtime to slow |
| | through on something | down and signal to your body |
| | that was | that it's time for bed, and then |
| | important to you? Did | sleep should come more |
| | you connect | naturally. |
| | well with the people | Try to give yourself permission to |
| | around you? | let go of negative |
| | Make a list of | thoughts and worries as well. |
| | things that were | Repetitive negative thinking, |
| | impactful that day and a | doing activities, and working close |
| | list of things | to bedtime can result in |
| | to think about | a longer period of time needed to |
| | or deal with tomorrow | fall asleep. |
| | so you don't lie awake | Bright lights from electronics can |
| | thinking about them. | also delay sleep, so it's |
| | | best to turn them off a few hours |
| | | before you go to bed and |
| | | keep them out of the bedroom. |
| R-04 | (Several task variations | Many people who don't get enough |
| | can fall under | sleep prioritize other |
| | this activity, or they can be | things instead, like work, |
| | combined:) | socializing, or relaxing. Millions |
| | Task 1: (You Decide How) | of others suffer from insomnia, a |
| | If you're | very common sleep |
| | having trouble falling | disorder where people can't fall |
| | asleep at night | asleep or stay asleep, no |
| | and it's been 20 minutes or | matter how hard they try. |
| | more, put | Whatever the reason, there are |
| | on a comfy robe or | many strategies we can use |
| | blanket, keep the | to help us get all the sleep we |
| | lights low, and go do | need. |
| | something | One way is to increase sleep |
| | relaxing. This could be | efficiency. Sleep efficiency |
| | reading, | refers to how well you're using |
| | writing in a journal, or | your time in bed. People |
| | petting your | with a low sleep efficiency are |
| | dog or cat. The point is to | spending a lot of time in bed |
| | change your | not sleeping. If you decrease the |
| | environment so that you | amount of non-sleeping |
| | are practicing | time you spend in bed, you might |
| | stimulus control-meaning | just become a more |
| | sending | efficient sleeper. Helping yourself |
| | yourself the correct | to fall asleep sooner by |
| | messages, like | getting enough exercise during the |
| | bed is for sleeping, not | day, eliminating |
| | for being | caffeine, and putting devices away |
| | awake. Do this every night | early in the evening can |
| | and notice | also increase your sleep efficiency. |
| | how soon you are able to | Stimulus control is also |
| | fall asleep faster. | important. |
| | Task 2: Turn Back Time: | Stimulus control was developed |
| | Clocks are a | based on the core principles |
| | basic necessity throughout | of classical conditioning. The idea |
| | our day, | is that your body can |
| | but when it comes to sleep, | learn a response to being in a |
| | it's best to | specific situation, especially if |
| | clock out. Try moving | that response is consistently paired |
| | your clock out | with that situation. For |
| | of sight at bedtime, and if | example, if the bed is for sleep |
| | you have to | only, getting into bed will |
| | wake up at a certain time | trigger a sleep response. However, |
| | in the | when other activities |
| | morning, set an alarm and | occur in bed, like working on your |
| | don't look | laptop, sleep is no longer |
| | at your clock until it goes | the triggered response. Instead, no |
| | off If you | specific response is |
| | wake up in the night, | triggered, or an alternative one is |
| | don't worry | (such as thinking about |
| | about what time it is, and | work). Stimulus control is a |
| | don't even | powerful technique and an |
| | check. Just take some | effective way to improve sleep, |
| | relaxing deep | reduce insomnia symptoms, |
| | breaths and go back to | and improve sleep efficiency. |
| | sleep, however | Why is it so important to stick to a |
| | long that might take. | regular bedtime and |
| | Task 3: Time and Again: | wakeup schedule? A 2019 study |
| | Try to wake | found that getting different |
| | up at the same time every | amounts of sleep each night can |
| | day, even | put you at higher risk for |
| | on your days off If your | obesity, hypertension, high |
| | alarm for | cholesterol, and other metabolic |
| | work normally goes off at | disorders. |
| | 6:30, for | [Clock tyranny:] When people |
| | example, get up at | wake in the middle of the |
| | 6:30 on the | night, one of the first things they |
| | weekends, too, but do | typically do is to check the |
| | something | time. But this is actually making |
| | enjoyable. Make | things worse for yourself |
| | coffee, go for a walk | because frequent clock-checking |
| | or to sunrise yoga, or read | can interfere with sleep. |
| | a book and | Try to make the clock (mostly) |
| | then plan out your day. | inaccessible, to reduce the |
| | You can turn | temptation to check it. Maybe put |
| | those early morning hours | your phone in a drawer or |
| | into some | turn the bedside clock away from |
| | epic “you” time! | you. This way, you won't |
| | Task 4: Night Owl or Early | get that nocturnal rush of brain |
| | Bird?: Do | activity that could make it |
| | you ever describe yourself | more difficult to return to sleep. |
| | in terms of | [Wake up at same time each day:] |
| | when you're at your best, | If you can always wake |
| | such as | up within about one hour of your |
| | “I'm a night owl” or “I'm | usual wake time every |
| | an early | day, rather than waking up at all |
| | bird”? It turns out there's | different times, you'll sleep |
| | something | much better at night and feel better |
| | to it, and it's actually | during the day. Why? |
| | beneficial to just | Because your brain likes routine, |
| | roll with it. If your eyes get | and it's a fabulous host |
| | heavy at | that gets things all ready for you |
| | 9:30pm, for example, | before you wake and |
| | great! It's time | before you sleep. Think of it this |
| | for bed. If you're the type | way: Your brain is like |
| | that just hits | that friend who throws a fantastic |
| | their stride in the evening | dinner party and has all |
| | and you | the food and drinks set out for you |
| | groove until midnight, | before you arrive so you |
| | great! Do your | can have a great time while you're |
| | thing and head to bed | there. But instead of |
| | when you're | food and drinks, your brain sets out |
| | tried. Try matching your | super helpful chemicals, |
| | sleep and | like cortisol to help you wake up |
| | wake times to your natural | and feel awake, or |
| | preference, | melatonin to help you wind down |
| | and you'll be more in sync | and feel sleepy. If you |
| | with your natural self. | show up to the dinner party too |
| | Task 5: There's a Nap for | early or too late, the host |
| | That: Guess | won't be ready for you and the |
| | what: napping is allowed! | party (a.k.a. how you feel |
| | If you feel | that day) will flop. |
| | yourself getting low on | [Night owl or early bird:] Science |
| | energy in the | strongly supports that |
| | middle of the day, find a | people are different when it comes |
| | comfortable | to their best time of day. |
| | place to lie down if you | Those who think faster and feel |
| | can, pull a | better in the evening are |
| | soft blanket over you, and | often called “night owls” while |
| | nap away! | those who do best in the |
| | A brief nap early in the day | morning are called “early birds.” |
| | can be a | Night owls can survive as |
| | great way to boost your | early birds, but they tend not to |
| | energy and | flourish. If your best |
| | reduce fatigue. Try to | performance is in the evening, try |
| | keep your | to adapt your schedule so |
| | midday zzzz to under 40 | you can be awake at those times. |
| | minutes and | [Napping:] A relatively brief nap |
| | lie down as close to | can improve physical |
| | midday as | performance, reduce fatigue, and |
| | possible. (The later it is, | improve mental |
| | the more | performance. Naps can also help |
| | trouble you might have | improve learning and |
| | falling asleep | memory. The length of the nap |
| | at bedtime.) | doesn't seem to matter, as |
| | | long as it's kept to less than an |
| | | hour and is early in the day. |
| | | Too much napping, however, is a |
| | | no-no: it will likely throw |
| | | off your internal clock and make |
| | | your sleeplessness worse |
| | | the next night! |
| R-05 | You should probably sit | You should probably sit down for |
| | down for | this. Actually, scratch |
| | this. Actually, scratch | that, you might want to stand up |
| | that, you might | for this news: sitting for |
| | want to stand up for this | long periods is really bad for you. |
| | news: sitting | Most of us do it because |
| | for long periods is really | we have desk jobs, so it makes |
| | bad for you. | sense how we got here. But |
| | Most of us do it because | we need to break the habit and take |
| | we have desk | a stand. Try this: While |
| | jobs, so it makes sense | sitting at work or at home, |
| | how we got | whether you're watching TV, |
| | here. But we need to break | listening to music, reading or |
| | the habit | performing desk work, make a |
| | and take a stand. | point to stand up for 3 to 5 minutes |
| | Try this: While | every horn. You can |
| | sitting at work or at | continue watching, listening, or |
| | home, whether | reading/working while |
| | you're watching TV, | standing, to minimize the |
| | listening to | interruption to your activity. |
| | music, reading or | |
| | performing desk | |
| | work, make a point to | |
| | stand up for 3 | |
| | to 5 minutes every hour. | |
| | You can | |
| | continue watching, | |
| | listening, or | |
| | reading/working while | |
| | standing, to | |
| | minimize the | |
| | interruption to your | |
| | activity. | |
| R-06 | To calm any stress and | Performing diaphragmatic |
| | anxiety you | breathing has many benefits, |
| | might be feeling, | including reducing anxiety, stress, |
| | connect with your | and cortisol levels (which |
| | breath by | is the hormone we release under |
| | performing diaphragmatic | stress). It also reduces heart |
| | breathing | rate and breathing rate and |
| | (DB) exercises | increases melatonin levels, |
| | throughout the day. | which help us sleep. What's more, |
| | This means breathing | diaphragmatic breathing |
| | deeply so that | has been shown to increase our |
| | when you | ability to pay attention and |
| | inhale, your belly | also help reduce negative thoughts, |
| | expands-rather | which are often the |
| | than your chest- | result of stress and anxiety. Talk |
| | allowing you to breathe | about a powerful defense |
| | more fully. Notice how | mechanism! |
| | you're feeling before the | |
| | exercise, and then | |
| | check in again | |
| | after. The simplest | |
| | technique involves | |
| | inhaling through | |
| | your nose for a count | |
| | of 3 seconds, holding | |
| | for 4 seconds, | |
| | and exhaling for 3 seconds. | |
| R-07 | When it comes to exercise, | A recent study showed that |
| | every little | sporadic walking or moderate- |
| | bit helps. And you can get | to-vigorous physical activity of |
| | your little | any duration, including |
| | bits in more easily than | bouts as short as 5 minutes, |
| | you think. For | improves our overall health and |
| | example, set an alarm | helps us live longer. You can do 5 |
| | once in the | minutes! And a 30- |
| | morning between 9am and | minute morning walk was found to |
| | 12pm and | be as powerful as |
| | once again between | medication when it came to |
| | 1pm and 5pm | lowering the blood pressure of |
| | during the workday. | sedentary older adults. Walking |
| | When your alarm | too boring for you? |
| | goes off stand up from | Researchers also found that |
| | your desk and | swapping even half an hour of |
| | go for a brief 3-5 minute | sitting for some type of physical |
| | walk. (If | activity of any intensity |
| | you're able to, you can | can reduce your risk of early |
| | lengthen the | death by 35 percent. |
| | time of your walk or | Even those least inclined to be |
| | recruit a | active and at the greatest risk |
| | “walking buddy” to do it | for developing chronic disease |
| | with you.) | can benefit from performing |
| | [More difficult version:] | short bouts (5-10 minutes) of |
| | Move on the | moderate walking to improve |
| | Job: If you want more | health outcomes. |
| | of a challenge, | The benefits of walking are pretty |
| | try this: During the day, | significant, and you don't |
| | incorporate | have to do as much as you think. |
| | two brief walking | For example, one study |
| | excursions into | sought to find the impact of a 100- |
| | your routine. First, you | day, 10,000-step program |
| | could park | in 1,963 people, measuring signs |
| | farther away from | of depression, anxiety and |
| | your office than | stress, as well as general well- |
| | you normally do and | being. At the end of the 100 |
| | take the extra | days, all measures of mental |
| | steps into work. For | health were improved |
| | your second | regardless of how many steps a |
| | excursion, walk to a | person walked over the 100- |
| | co-worker in | day period. Interestingly, the |
| | another building or | disparity between steps was |
| | on a different | pretty great, and all with the same |
| | floor to stay hi, instead of | outcome: some people |
| | calling them. | walked an average of 2,775 |
| | [Advanced versions:] | steps, while others walked as |
| | Track Your | many as 112,831 steps. This study |
| | Moves: Track your steps | confirms that you don't |
| | using a | need to walk 10,000 steps a day, |
| | pedometer, accelerometer, | as many people believe, to |
| | smartwatch, or smart | get the mental health benefits of |
| | phone, and set a | walking. ~R-19 A 2019 |
| | goal of steps each day. | study of over 16,000 senior |
| | The number of | women also found that those |
| | steps can range from an | who walked just a moderate |
| | average of | amount (an average of just |
| | 2775 or higher. You can | under 4,400 steps a day) were 41 |
| | also run, | percent less likely to die |
| | cycle, or get your | over the next four years than |
| | heart pumping in | women who walked around |
| | any other way you choose. | 2,700 steps a day. |
| | | Aerobic exercise is beneficial for |
| | | your brain, too-a recent |
| | | study found that adults assigned to |
| | | a 6-month aerobic |
| | | training program significantly |
| | | improved their cognition and |
| | | executive function. |
| R-08 | Stretching is like the | Stretching can be done almost |
| | dessert portion | anywhere, and it is |
| | of fitness routines. | surprisingly good for you. |
| | For 5 to 7 minutes | Moreover, the benefits aren't |
| | each day, have your | only physical: for example, 30 |
| | dessert by | minutes of stretching |
| | performing a simple | exercises can enhance your mood |
| | series of large- | and make you think a |
| | muscle, whole-body | little faster and even improve your |
| | stretches, using | reaction time. One study |
| | traditional stretches | showed that a 12-week yoga |
| | found in yoga, | intervention resulted in |
| | qigong, and tai chi. | increased brain thalamic GABA |
| | These stretches | levels, which improves our |
| | will get your | mood and decreases anxiety levels. |
| | blood flowing into | Yoga stretches also help |
| | places that may have | to regulate our hormones. |
| | been blocked by | |
| | tight muscles. | |
| R-09 | Engage your core! These | Currently, low back pain (LBP) |
| | are three of | affects more than 80% of |
| | our favorites, which you | people at some point in their life |
| | can-and | and often results in lost |
| | should-do several times | wages, additional medical |
| | a week. | expenses, and a risk of |
| | The first exercise is | developing other medical/health |
| | cat-cow | conditions. In the United |
| | performed on your hands | States alone, total indirect and |
| | and knees | direct medical costs of LBP |
| | with your back straight, | exceed $100 billion every year. A |
| | hands under | number of studies |
| | your shoulders, and knees | confirm that core/trunk |
| | under your | stabilizing/balancing exercises are |
| | hips. Begin by arching | proven very effective for helping |
| | your back up | to treat and prevent LBP. |
| | and hold for 3 | Some of the research shows that |
| | seconds, and then let | performing these exercises |
| | your back/abdomen sag | for as little as a week is effective. |
| | to the floor | Additionally, even in healthy |
| | and hold for 3 seconds. | adults, integrating core |
| | Perform 5 sets. | stability training into your exercise |
| | The second exercise | regimen may help to |
| | starts in the same | prevent injury, particularly in the |
| | cat-cow position (hands | lower extremities (think |
| | and knees on | hips, knees, and ankles). |
| | floor). Begin by | |
| | extending your | |
| | opposite upper and | |
| | lower limbs | |
| | straight out (for example, | |
| | left leg | |
| | behind, right arm in | |
| | front); maintain | |
| | for 30 seconds and | |
| | switch sides. | |
| | The third exercise is a | |
| | spine twist It | |
| | begins with kneeling | |
| | on a pillow with | |
| | your arms extended out to | |
| | your sides. | |
| | Rotate your trunk, | |
| | head and arms to | |
| | one direction and hold for | |
| | 30 seconds | |
| | and then rotate to the | |
| | other side. | |
| | Repeat 2 times. | |
| R-10 | Plan on signing up for a | A fascinating study using |
| | fitness class | temptation bundling showed that |
| | (including yoga, tai | when exercise is bundled with |
| | chi, pilates, | tempting audio book novels, |
| | aquatics, or ballroom | it increased college students' gym |
| | dancing), ideally | attendance by 51 percent! |
| | one that you can | The study also showed that for |
| | commit to with some | people with self-control |
| | co-workers, a partner, | challenges, temptation bundling |
| | or friends. It | was especially effective by |
| | can be first thing in | offering a low-cost solution to |
| | the morning, at | two common willpower |
| | lunch, right after work, | problems of under engagement |
| | or in the | (not engaging in a “should” |
| | evening. The important | behavior like exercise, often |
| | things are to | enough) and over engagement |
| | get moving, do something | (indulging in “want” behaviors |
| | you enjoy, | too often). |
| | and do it with others so | Ultimately, having the motivation |
| | you're held | to perform exercise is |
| | accountable. If you | essential to forming a long-term |
| | find it really | habit, and this is driven |
| | challenging to exercise | primarily by the self-determination |
| | in general, | theory, which focuses |
| | incentivize your | on personality factors and the |
| | commitment to the | surrounding environment of |
| | class with what's | each individual. Choose your |
| | called “temptation | exercise companions wisely |
| | bundling”. This | because they will greatly influence |
| | involves linking an | your likelihood of |
| | instant gratification | sticking with the exercise long |
| | “want” activity, | term. Of course, this also |
| | such as watching the | influences your satisfaction and |
| | next episode of | enjoyment of the exercise |
| | your favorite TV show, | itself! |
| | listening to | |
| | your favorite | |
| | podcast, reading a | |
| | chapter from a book | |
| | you're loving, or | |
| | receiving a massage, | |
| | with a “should” | |
| | behavior that provides | |
| | a long-term | |
| | health benefit but | |
| | requires energy and | |
| | willpower to complete, | |
| | such as | |
| | exercising. In short, do | |
| | the “should” | |
| | to get the “want.” | |
| | For example, you could | |
| | listen to your | |
| | favorite album while you | |
| | run on the | |
| | treadmill, go to | |
| | your favorite | |
| | restaurant with friends | |
| | after your | |
| | pilates class, or | |
| | listen to your | |
| | audiobook on your | |
| | way home from | |
| | yoga. The bundle | |
| | options are endless! | |
| R-11 | Try boosting your workout | Resistance/strength training |
| | routine a | improves not only your |
| | little to see what | physical strength and performance |
| | differences you | abilities, but also your |
| | notice. Combine a | cognition and executive function. |
| | resistance exercise, | This is true for people |
| | like lifting weights, | across age groups, including older |
| | using resistance | men and women. ~R-28 |
| | bands, or even water | One study showed resistance |
| | exercises, with | training twice a week results |
| | your whole-body | in the most favorable changes to |
| | workout routine 2 to | quality of life and sense of |
| | 3 times a week to | coherence (feeling optimistic and |
| | increase your | in control) among men |
| | strength, improve | and women 65 to 75 years old. |
| | your overall | Additional research showed that |
| | wellness, and | resistance training |
| | enhance your thinking | improves body composition |
| | abilities. Does it | (reduced fat mass, increased |
| | make you feel | lean body mass), muscle strength, |
| | stronger, faster, | and physical function in |
| | and happier? | the obese elderly, whether the |
| | | individual has changed their |
| | | diet or not. |
| | | It can also give your mental health |
| | | a boost. A meta-analysis |
| | | of 33 randomized controlled trials |
| | | found that resistance |
| | | exercise training was associated |
| | | with a significant reduction |
| | | in depressive symptoms. |
| R-12 | Figure out your ideal | Committing to a comprehensive |
| | fitness routine- | fitness routine has many |
| | you know, the one that | proven physical and psychological |
| | makes you feel | benefits-it gives us a |
| | glowy and awesome | feeling of accomplishment, helps |
| | head-to-toe | us reduce stress, and |
| | afterward-and make a | promotes good sleep. In one study, |
| | commitment | people who did low, |
| | to stick with it. This is | moderate, or high levels of |
| | your jam! | physical activity were assessed |
| | Choosing the most | for changes in mood and levels of |
| | appropriate fitness | brain activation of |
| | routine is a personal | pleasure-seeking receptors. It turns |
| | decision that | out all levels of physical |
| | takes into account | activity increased brain receptors |
| | preferences, goals, | of pleasure and enhanced |
| | feasibility and time. For | mood improvements. In other |
| | example, you | words, regardless of the level |
| | might love how free | of physical activity/exercise you |
| | weights, boxing, | perform, you will likely |
| | and spin class make you | feel happier. |
| | feel. Find the | Additionally, when Yale and |
| | best times of day and | Oxford researchers collected |
| | the most | data on over 1 million individuals, |
| | convenient locations for | they found that people |
| | you to do | who exercised regularly tended to |
| | these favorite activities, | be happier. On average, |
| | and make it | regular exercisers felt bad for 35 |
| | part of your daily | days a year, whereas |
| | routine. Period. | nonactive individuals felt bad for |
| | Fitness and performance | 53 days, on average. |
| | training are | What's more, they found that the |
| | powerful: they enhance | active group felt just as |
| | your mood | happy as nonactive individuals |
| | and make you feel | who earned about $25,000 |
| | stronger, which in | more a year. In other words, one |
| | time will make you | has to earn a lot more to |
| | want to keep doing it. | get the same happiness boost from |
| | | regular exercise and |
| | | sports. ~R-36 No need to overdo it, |
| | | either. The same study |
| | | found that when it comes to better |
| | | mental well-being, three |
| | | to five training sessions per week, |
| | | each between 30 to 60 |
| | | minutes, are ideal. People who |
| | | exercised for more than three |
| | | hours a day actually had lower |
| | | mental health than people |
| | | who weren't particularly active. |
| | | Regular exercise has also been |
| | | shown to be an effective |
| | | treatment for depressive |
| | | disorders by itself and when |
| | | combined with other therapies. |
| | | Remember, there's no one |
| | | size fits all when it comes to an |
| | | ideal exercise routine. What |
| | | works for someone else may not |
| | | work for you, so take stock |
| | | of your capabilities and |
| | | preferences as you craft a routine |
| | | that's ideal for you. |
| R-13 | Water is essential to life, | If you don't tend to drink much |
| | but do you | water, like less than about a |
| | know what kind of life is | liter a day, doubling your water |
| | waiting for | intake can significantly help |
| | you if you drink more of | improve any feelings of fatigue, |
| | it? Increased | confusion, thirst, or |
| | water intake may lower | sleepiness you might have. One |
| | your risk of | study showed that doing the |
| | depression and anxiety, | opposite-cutting water intake in |
| | and even | half for people who |
| | improves cognitive | already drink enough water-had |
| | function. Keep a | disastrous effects: it |
| | water bottle fdled with you | increased thirst and decreased |
| | wherever | feelings of contentedness, |
| | you go, and drink, drink, | calmness, positivity, and vigor. |
| | drink. In | Don't do that! |
| | general, you should try to | A related study showed that for |
| | drink | every one percentage point |
| | between half an ounce and | increase in daily water intake, the |
| | an ounce | quality of people's diets |
| | of water for each pound | improved drastically. Lowering |
| | you weigh, | your water intake, however, |
| | every day. We know. It's a | down to less than two glasses of |
| | lot. But | water per day, may double |
| | you get used to it quickly, | your risk of depression and |
| | and you can | anxiety. How amazing is it that |
| | make it extra delicious | getting enough water could protect |
| | by adding | you against these |
| | electrolyte tablets, or slices | conditions? |
| | of lemon, | |
| | lime, cucumber, or a | |
| | handful of berries. | |
| R-14 | Make your mornings a | A simple, high-quality source of |
| | significant part | protein for breakfast, like |
| | of your self-care routine | eggs, peanut butter, or yogurt gets |
| | by starting | the day off to an |
| | your day with a nutrient- | energetic start and has been shown |
| | dense meal | to help reduce body fat, |
| | containing a high-quality | make you eat less during the |
| | source of | day, help you to feel less |
| | protein and fiber. For | hungry overall, and may even |
| | example, eat | improve your sleep quality. |
| | foods like a bowl of | Regularly skipping breakfast |
| | oatmeal with a | isn't such a great idea. A |
| | 1/2 cup of Greek | recent study among college |
| | yogurt and | students showed that skipping |
| | nuts/seeds sprinkled on | breakfast is associated with being |
| | top, or eggs | less happy. -R-42 |
| | with multigrain toast and | Whereas in adults, eating breakfast |
| | a slice of | showed a robust |
| | avocado, or a bowl of acai, | advantage for memory |
| | chia, flax, | (particularly delayed recall), |
| | almond butter and sliced | and for |
| | banana, or | adolescents, tasks requiring |
| | high-protein cereal with | attention, executive function, |
| | whole grains | and memory were enhanced by |
| | and fresh fruit. A protein | eating breakfast (compared |
| | smoothie is | with skipping it). |
| | also a delicious way to | |
| | start the day | |
| | and is always a hit with | |
| | kids and those on the go. | |
| | Try to gather the meal | |
| | together the | |
| | night before so it's | |
| | ready and waiting | |
| | for you to assemble in | |
| | the morning. | |
| | Once you get into this | |
| | high-protein | |
| | morning habit, you may | |
| | find yourself | |
| | controlling your weight | |
| | better, losing | |
| | body fat, feeling less | |
| | hungry during | |
| | the day, and even sleeping | |
| | better at night! | |
| R-15 | It's amazing how much | The Mediterranean diet is high in |
| | lighter and | fruits, nuts, greens, fish, |
| | better you can feel just | and healthy fats and can reduce |
| | by altering | your risk for depression. No |
| | your diet to accommodate | sea kelp shakes here, just tasty, |
| | fresh foods. | healthy food to enhance |
| | Aim to incorporate into | your mood. A 2016 study also |
| | your diet five | found that increasing your |
| | servings a day of healthy | intake of fruits and vegetables is |
| | veggies and | linked to greater happiness |
| | fruit, and a handful of nuts | and well-being, even after |
| | and seeds. | adjusting for other factors. |
| | If you're not eating any | This way of eating is also great for |
| | of these | the rest of your body. A |
| | things at all, start by | comprehensive meta-analysis of |
| | consuming one | over 2 million people |
| | serving of fruit, greens | showed that eating more fruits and |
| | (veggies), and | vegetables led to reduced |
| | nuts during the day. | risk for cardiovascular disease, |
| | A few simple strategies | cancer, and mortality in |
| | include 1) | general. This study also found that |
| | Making a breakfast | fewer than 5 servings of |
| | protein smoothie | fruit and 8 servings of veggies per |
| | with some berries and | day increases the risk for |
| | scoop of | these conditions. |
| | nut/seed butter. 2) | |
| | Including a salad or | |
| | some colorful raw or | |
| | steamed veggies | |
| | at lunch and dinner and | |
| | 3) Having | |
| | fresh-cut veggies with | |
| | hummus or | |
| | Greek yogurt dip for a | |
| | snack. 4) | |
| | Keeping nuts/seeds and | |
| | dried fruit in | |
| | your car or bag so you | |
| | can have a | |
| | handful when you | |
| | need an energy boost. | |
| R-16 | Healthy snacking is all | Eating and drinking too much |
| | about choosing | sugar may make you feel |
| | foods and beverages with | physically unwell in the short term |
| | the lowest | and is associated with an |
| | simple sugar content | increased risk of diseases like Type |
| | because our | 2 diabetes and |
| | body and mind function | cardiometabolic diseases. |
| | better with | High sugar intake may even lead to |
| | less sugar intake. You're | common mental |
| | in Revive mode, so | disorders, such as depression. In |
| | choosing healthy, protein- | fact, risk for depression is |
| | rich, no-sugar foods and | significantly elevated in men and |
| | drinks are | women who consume |
| | vital to nourishing your | large amounts of sugar; it elevates |
| | body and | to almost 23 percent over |
| | growing your vitality | 5 years! This is especially true for |
| | and energy. | postmenopausal women. |
| | Here are a few snack | People who eat the right stuff, |
| | hacks that might | however, like the fiber, fruits, |
| | transform your afternoons: | and veggies experts recommend, |
| | Make a small batch of | have much lower incidents |
| | hard-boiled | of depression. So stick to this way |
| | eggs and take one with | of eating, and you're on |
| | you when you | the right track all around. |
| | go out in the morning, | Our snacking recommendations are |
| | along with | also on point: One |
| | fresh sticks of carrot, | recent study showed that eating |
| | peppers, beans, | high-protein snacks |
| | cucumber, and any other | throughout the day helped to |
| | raw veggie | satisfy hunger in |
| | you love. Pair that | overweight/obese men, which |
| | with some | means it likely reduced their |
| | homemade hummus | chances of overeating at meal |
| | (it's so fast to | times or turning to the wrong |
| | make!) and you have | foods. |
| | yourself a tasty, | |
| | healthy snack. | |
| | For your protein boosts, | |
| | you can do a | |
| | few things: Make | |
| | homemade trailmix | |
| | with nuts/seeds and | |
| | dried fruit; buy | |
| | some grass-fed jerky, | |
| | available in | |
| | most grocery stores or | |
| | order online; | |
| | pick up some organic, | |
| | pasture-fed | |
| | Greek yogurt (which tends | |
| | to be lower | |
| | in sugar than other | |
| | yogurts) or cottage | |
| | cheese and top with berries | |
| | and chia or flax seeds. | |
| R-17 | One of the hardest | Preparing your meals ahead of |
| | things about | time is a sure way to choose |
| | changing your diet is the | foods wisely for yourself, and it |
| | meal prep. | can also lead to weight loss. |
| | You're not used to cooking | In fact, a study of over 1,000 adults |
| | with those | showed that those who |
| | ingredients yet, and | planned meals ahead of time, such |
| | having to make | as preparing meals the |
| | something diet compliant | night before, had a greater |
| | every night | likelihood of weight loss |
| | gets a little daunting | compared to those who didn't |
| | and tiresome. So | prepare meals. If you think |
| | do yourself a really big | about it, it makes sense. Eating in a |
| | favor and feed | restaurant or grabbing |
| | two birds with one stone. | food on the go usually means high- |
| | Each time you cook a | fat and high-sugar |
| | recipe at night, | options, and you're not in a |
| | make enough for at | position to choose fuel that your |
| | least 2 to 3 meals | body needs most. If you're thinking |
| | so you have leftovers | about what you want to |
| | for lunch the | put in your body before you're |
| | next day or for dinner | even hungry, your choices |
| | the following night. | will make much more sense. |
| | You can also pack your | |
| | snacks ahead | |
| | of time in “go bags” | |
| | the night before | |
| | so you don't have to think | |
| | too hard when you're | |
| | running late. Just | |
| | remember to include | |
| | fresh veggies, | |
| | fruits, nuts, and | |
| | proteins in bags or | |
| | containers so they're | |
| | ready as you | |
| | walk out the door. | |
| S-01 | Are you savoring an | Studies show that people who |
| | incredible meal, | often appreciate and savor |
| | a hike through the | what they have-their possessions, |
| | woods, or a | traits, relationships or |
| | gorgeous sunset? Whatever | accomplishments-tend to |
| | it is, focus | experience more happiness than |
| | on the details, let | people who don't. |
| | yourself get totally | When mindfulness becomes a |
| | immersed, and use all of | habit, the neural pathways in |
| | your senses | our brains linked to positive |
| | to intensify and | feelings become stronger and |
| | prolong your positive | more active, increasing the chances |
| | experience. | we will continue to feel |
| | Upload a photo so you | happier in the future. |
| | can savor the | And that's just for starters. |
| | memory of it later! | Researchers ~S-2 have also |
| | | found that savoring makes us feel |
| | | more grateful and |
| | | hopeful, which gives us more self- |
| | | confidence. And when |
| | | we feel better about ourselves, |
| | | we're more open to others. |
| | | People who savor are also less |
| | | likely to experience |
| | | depression, stress, guilt, |
| | | shame and depression. |
| S-02 | Block out 10 minutes, | A 2018 study published in the |
| | lie down | journal Psychosomatic |
| | somewhere comfortable, | Medicine found that body scan |
| | and spend a | mediation decreases stress |
| | minute focusing on | and increases levels of |
| | your breathing. | mindfulness. In addition, several |
| | Now, imagine you're | studies have shown that when |
| | taking a scan of | practiced regularly, |
| | your whole body, starting | meditation has the potential to |
| | with your | help lower high blood |
| | feet and moving | pressure and lessen chronic pain, |
| | past your torso on up | anxiety and depression. It |
| | to your head. | can also alter the regions of the |
| | Just “feel” each body part | brain associated with |
| | as you scan | memory, self-awareness and |
| | over it, then allow it to | compassion. |
| | fade and move | In a study conducted at the |
| | on. Dismiss any thoughts | Massachusetts General Hospital, |
| | that stray | participants attended weekly 2.5- |
| | from your focus. | hour group meetings in |
| | | which they practiced mindfulness |
| | | meditation. At the end of |
| | | the 8 weeks, the meditators felt |
| | | more capable of acting with |
| | | awareness, observing the world |
| | | around them and remaining |
| | | nonjudgmental. So how exactly |
| | | does meditation reduce |
| | | anxiety? In a 2013 study, |
| | | scientists at Wake Forest |
| | | identified the brain functions |
| | | involved in the process. While |
| | | meditating, participants showed |
| | | greater brain activity in the |
| | | ventromedial prefrontal cortex, |
| | | the area that controls |
| | | worrying. And when activity |
| | | increased in the anterior |
| | | cingulate cortex (the part of the |
| | | brain that controls thinking |
| | | and emotions), anxiety levels also |
| | | decreased. The best part, |
| | | at least for beginner meditators? |
| | | You don't have to sit for 30 |
| | | minutes to reap the benefits: |
| | | According to the lead author of |
| | | the study, Fadel Zeidan, Ph.D, |
| | | “just a few minutes of |
| | | mindfulness meditation can help |
| | | reduce normal everyday |
| | | anxiety.” |
| S-03 | Come up with something | In a study about savoring |
| | you can | strategies, researchers found that a |
| | enjoy doing with | savoring strategy called |
| | someone else, and | capitalization, or sharing, enhances |
| | savor your experience, | our life satisfaction and increases |
| | then discuss it | our everyday happiness, |
| | afterwards. It | over and above the impact of the |
| | could be a shared meal, | positive event itself. |
| | a walk through a beautiful | They say the best things in life are |
| | park, or a piece of good | meant to be shared, and |
| | news you have received. | recent research proves this to be |
| | Be in the moment, enjoy | true! Yale researchers |
| | the present | recently found that when people |
| | and be mindful of | pay attention to the same |
| | everyone's joy. | pleasant thing, whether it's a song, |
| | Add a photo too! | a dessert, or a scenic |
| | | view, the experience is much more |
| | | pleasurable. In other |
| | | words, we get more joy out of |
| | | experiences when they're |
| | | shared than we do alone. |
| S-04 | Today, revisit a good | Dr. Fred Bryant (who coined the |
| | memory from | term “savoring”) says that |
| | your past and take | savoring can be “time-shifted,” |
| | the time to savor it. | meaning it can be enjoyed |
| | Reminisce about a | in the past, present and future. |
| | happy childhood | Some like to savor the past |
| | event, a great vacation, | through reminiscing, others savor |
| | a joyful family | the future through the |
| | event, a personal victory, | anticipation of things to come, and |
| | a rewarding | others enjoy just being in |
| | accomplishment. | the present. |
| | Use your senses to | Through his research, Dr. Bryant |
| | put yourself there. | has also confirmed that |
| | Think about how it | savoring is beneficial to our health |
| | looks, smells, | and happiness. People |
| | tastes. How do you | who regularly and frequently savor |
| | feel? Report back | are happier, less |
| | and describe your savoring | depressed, more optimistic and |
| | experience. Add | more satisfied with life in |
| | a photo too! | general. |
| | | Each form of savoring has its own |
| | | benefits. People who |
| | | savor the present are less prone |
| | | to depression, have less |
| | | stress, guilt, and shame. Those |
| | | who savor the future |
| | | (anticipation) are more optimistic. |
| | | And people who savor |
| | | the past (reminiscing) are best able |
| | | to buffer stress. In fact, a |
| | | 2017 study conducted at Rutgers |
| | | University found that |
| | | recalling positive memories could |
| | | combat acute stress at a |
| | | physical level. Brain scans |
| | | showed that it was associated |
| | | with increased activity in |
| | | prefrontal brain regions associated |
| | | with emotion regulation and |
| | | cognitive control-the same |
| | | regions suppressed by acute stress- |
| | | as well as in the regions |
| | | associated with “reward” |
| | | processing. |
| S-05 | When faced with a | Breathe a sigh of relief: |
| | difficult decision | Psychologist and author Robert |
| | or an upcoming challenge, | Leahy, Ph.D., of Weill Cornell |
| | we often | Medical School, found that |
| | overthink or over- | 85% of the stuff we worry about |
| | complicate the | have positive or neutral |
| | situation to the | outcomes. And even when our |
| | point where we end up | worries do become a reality, |
| | doing nothing. | about 80% of us say we handled |
| | So next time you start | the outcome better than we |
| | looping your | thought we would. |
| | negative tapes, create | Research by Matt Killingsworth |
| | a plan to distract | suggests that a wandering |
| | yourself or re-focus | mind is an unhappy mind-people |
| | yourself on the | are less happy when their |
| | task at hand. It can | minds are wandering and |
| | range from | distracted than when they're not- |
| | reciting a mantra to | especially if they're thinking about |
| | scheduling a | something unpleasant or |
| | block of “worry | even neutral. (Thinking about a |
| | time” each day, to | pleasant topic other than |
| | wearing a rubber band on | their current activity had no effect |
| | your wrist | on happiness one way or |
| | that you snap every | the other.) Mind-wandering also |
| | time you catch | occurs with higher |
| | yourself getting lost | frequency on tasks that don't |
| | in worries- | demand our full attention. |
| | anything that will | These activities are derived from |
| | help you to “reset” | Mindfulness-Based Stress |
| | your brain and derail | Reduction exercises, which range |
| | overthinking. | from mindfulness |
| | | meditation to being mindful during |
| | | stressful situations. |
| | | When practiced regularly, they can |
| | | improve coping with |
| | | distress and disability in our day to |
| | | day, as well as under |
| | | more extraordinary conditions of |
| | | serious stress. How? They |
| | | help us focus on what's happening |
| | | in the present moment, |
| | | rather than what happened in the |
| | | past, or what might happen |
| | | in the future. These activities also |
| | | allow us to recognize |
| | | (and distance ourselves from) a |
| | | distorted thought such as |
| | | “Everything's going to go wrong.” |
| | | Recognize it as simply |
| | | that-a thought-and then let it go. |
| S-06 | Sit with your spine | Studies show that meditation can |
| | upright but not | lower high blood pressure |
| | stiff, keeping your torso | and lessen chronic pain, anxiety |
| | centered and | and depression. Brain |
| | balanced. Feel your body | imaging studies show that |
| | from the inside. Pay | meditation actually alters regions |
| | attention to your | of the brain associated with |
| | breathing. The past is over | memory, self-awareness and |
| | and the | compassion. |
| | future isn't here | Beginner? No problem! |
| | yet. Just settle into | Researchers at the University of |
| | the present, the only | Wisconsin-Madison found that |
| | moment where | even beginners were able to |
| | we're truly living. | increase activity in the brain region |
| | Beginning meditators: | associated with positive |
| | Don't worry | thoughts, although experienced |
| | when your attention | meditators showed a greater |
| | wanders. Start | level of activity. |
| | with 5 or 10 minutes. And | In a study at Massachusetts |
| | keep in | General Hospital, participants |
| | mind: This is something | attended weekly 2.5-hour group |
| | you can do | meetings in which they |
| | whenever you have a few | practiced mindfulness meditation. |
| | minutes of free time. | At the end of the 8 |
| | | weeks, the meditators felt more |
| | | capable of acting with |
| | | awareness, observing the world |
| | | around them and remaining |
| | | nonjudgmental. So how exactly |
| | | does meditation reduce |
| | | anxiety? In a 2013 study, scientists |
| | | at Wake Forest |
| | | identified the brain functions |
| | | involved in the process. While |
| | | meditating, participants showed |
| | | greater brain activity in the |
| | | ventromedial prefrontal cortex, |
| | | the area that controls |
| | | worrying. And when activity |
| | | increased in the anterior |
| | | cingulate cortex (the part of the |
| | | brain that controls thinking |
| | | and emotions), anxiety levels also |
| | | decreased. The best part, |
| | | at least for beginner meditators? |
| | | You don't have to sit for 30 |
| | | minutes to reap the benefits: |
| | | According to the lead author of |
| | | the study, Fadel Zeidan, Ph.D, |
| | | “just a few minutes of |
| | | mindfulness meditation can help |
| | | reduce normal everyday anxiety.” |
| S-07 | Choose a low-energy | Being mindful requires you to be |
| | physical routine, | aware of physical |
| | such as yoga or gentle | sensations, perceptions, thoughts |
| | stretching, that | and imagery. Numerous |
| | takes about 20 minutes to | studies have shown that |
| | complete. | mindfulness meditation, especially |
| | Ideally, it should be | when practiced regularly, can |
| | something you | improve general coping with |
| | don't need to think about | stresses from everyday life, as |
| | too much. As you go | well as under more |
| | through each pose or | extraordinary conditions. |
| | stretch, pay close | According to studies, meditation |
| | attention to the | can also lower high blood |
| | effect it has on | pressure and lessen chronic pain, |
| | your body. Hold the | anxiety and depression. It |
| | stretch, focusing your | alters regions of the brain |
| | attention on every place | associated with memory, self- |
| | where you feel something | awareness and compassion. So |
| | in your body, one | how exactly does meditation |
| | place at a time. What's the | reduce anxiety? In a 2013 study, |
| | sensation like? Repeat this | scientists at Wake Forest |
| | for each pose. | identified the brain functions |
| | | involved in the process. While |
| | | meditating, participants showed |
| | | greater brain activity in the |
| | | ventromedial prefrontal cortex, |
| | | the area that controls |
| | | worrying. And when activity |
| | | increased in the anterior |
| | | cingulate cortex (the part of the |
| | | brain that controls thinking |
| | | and emotions), anxiety levels also |
| | | decreased. The best part, |
| | | at least for beginner meditators? |
| | | The benefits happen |
| | | quickly: According to the lead |
| | | author of the study, Fadel |
| | | Zeidan, Ph.D, “just a few minutes |
| | | of mindfulness |
| | | meditation can help reduce normal |
| | | everyday anxiety.” |
| S-08 | Deliberately arrange a day | According to researchers Dr. Fred |
| | of leisure. | Bryant and Joseph |
| | Fill your day with | Veroff, savoring involves noticing |
| | different types of | and appreciating the |
| | activities to savor-food, | positive things all around us. |
| | music, a | Savoring is the positive |
| | beautiful walk, | counterpart to coping. It's about |
| | or a visit to a | much more than mere |
| | museum-and savor | pleasure-it also involves |
| | each activity | mindfulness and “conscious |
| | using techniques | attention to the |
| | you've learned in | experience of pleasure.” |
| | previous levels, from | Researchers found that savoring |
| | mindfulness to | makes us feel more grateful |
| | capitalization to | and hopeful, which gives us more |
| | paying attention to | self-confidence. And |
| | the details. | when we feel better about |
| | | ourselves, we're more open to |
| | | others. People who savor are also |
| | | less likely to experience |
| | | stress, guilt, shame and depression. |
| | | In one study, researchers found |
| | | that a savoring strategy |
| | | called “capitalization,” or sharing |
| | | positive news with others, |
| | | enhances our life satisfaction and |
| | | increases our daily |
| | | happiness, over and above the |
| | | impact of the positive event |
| | | itself. |
| | | When mindfulness becomes a |
| | | habit, the neural pathways in |
| | | our brains linked to positive |
| | | feelings become stronger and |
| | | more active, increasing the chances |
| | | we will continue to feel |
| | | happier in the future. Now that |
| | | you've built up your |
| | | savoring skills, we think you're |
| | | ready to put your |
| | | mindfulness and capitalization |
| | | habits to work in an all-day |
| | | experience. Yes, it will be more |
| | | intense, and your savoring |
| | | moments will be more varied-but |
| | | you're ready for it! |
| S-09 | Each day this week, | One effective way to combat |
| | write down at least one | negative thoughts resulting |
| | unpleasant thing that | from an unpleasant experience is to |
| | happened to you, and | consciously challenge |
| | think about why | them by coming up with |
| | you think it happened. | alternative explanations for what |
| | Come up with | caused the event, an idea taken |
| | one explanation | from cognitive therapy. |
| | you're fairly | Martin Seligman also coined the |
| | confident about, | phrase “learned optimism” |
| | plus a few others. | in his book of the same name, |
| | Once you have a list | which states that people can |
| | of at least 3 | view negative experiences as |
| | possibilities (the | unlucky situations that are not |
| | bigger, the better), | personal in nature, |
| | read through each of | and aren't permanent. |
| | them. Which | In a study by researchers at the |
| | seems most plausible | University of Pennsylvania, |
| | to you? Which | college students at risk for |
| | seems least plausible? | depression were randomized into |
| | What evidence | an 8-week workshop group and a |
| | do you have in favor of | control group. Those in |
| | and against | the workshop group used |
| | each possibility? When | cognitive-behavioral techniques to |
| | this is done, | learn how to improve their |
| | go back to your original | explanatory style, which |
| | explanation. | included identifying automatic |
| | How sure do you feel | negative thoughts and |
| | about it now? | underlying beliefs, as well as |
| | | replacing negative thoughts |
| | | with more constructive |
| | | interpretations, which reduced |
| | | depressive and anxiety symptoms, |
| | | and improved well-being. |
| S-10 | Take a 30-minute walk | Being mindful requires you to be |
| | in a park-or | aware of physical |
| | anywhere with interesting | sensations, perceptions, thoughts |
| | scenery and | and imagery. Numerous |
| | activity. Walk slowly at | studies have shown that |
| | first, with | mindfulness meditation, especially |
| | eyes on the ground. | when practiced regularly, can |
| | Notice the tactile | improve general coping with |
| | sensations of walking: the | stresses from everyday life, as well |
| | feeling of | as under more |
| | your feet, the temperature, | extraordinary conditions. |
| | the way your body feels. | According to studies, meditation |
| | Once you can walk at a | can also lower high blood |
| | normal pace | pressure and lessen chronic pain, |
| | while paying attention to | anxiety and depression. It |
| | sensory | alters regions of the brain |
| | experiences, start | associated with memory, self- |
| | noticing sounds- | awareness and compassion. |
| | but try not to | The added benefit of doing a |
| | think too much! | walking meditation outside |
| | Once you can attend to | (especially in green spaces) is that |
| | tactile and | spending time in nature |
| | auditory sensations, | reduces stress and helps people |
| | start looking | feel energetic and more |
| | around and noticing | alive, according to scientists at the |
| | any activity around you. | University of Rochester. |
| | | A recent study using mobile EEG |
| | | devices to monitor |
| | | participants' emotions during a |
| | | walk also found that people |
| | | were more likely to experience |
| | | meditative-like brain waves, |
| | | and exhibit less frustration if they |
| | | were walking in a green |
| | | space, compared to a bustling |
| | | shopping street or a busy |
| | | business area. |
| S-11 | Decide on something to | Now that you've savored small |
| | savor with a | moments, you're ready to |
| | large group of people-a | savor longer experiences and |
| | community | events with others. In a study |
| | or neighborhood group, or | about savoring strategies, |
| | all of your | researchers found that |
| | coworkers, for example. | capitalization, or sharing, |
| | Perhaps you | enhances our well-being and |
| | can organize a group wine | increases our everyday happiness, |
| | tasting or a | over and above the |
| | class trip to a garden. | impact of the positive event |
| | Instruct the | itself. So your savoring event |
| | group on how to savor | may only last for a few hours, but |
| | the experience | its mood-boosting effects |
| | using what you've | could last for days. |
| | learned from | The research also suggests that |
| | practicing your social | sharing positive experiences |
| | savoring skills-take | may allow individuals to |
| | photos, note all the tiny | perceive themselves positively in |
| | details, and discuss what | the eyes of others, hence |
| | you value about the event | boosting self-esteem and |
| | and your time together. | facilitating positive appraisals of |
| | | one's life. Plus, scientists |
| | | know that people enjoy things |
| | | more when they do it with |
| | | others, and that spending quality |
| | | time with friends is a |
| | | highly effective mood-booster. |
| S-12 | Catch yourself when | Robert Epstein, PhD, says that we |
| | you're in the | have the power to stop |
| | middle of an unpleasant | stress before it even starts. |
| | experience. It | There's only a tenuous |
| | can be an emotional | relationship between stressors (the |
| | state (being | things that cause us to |
| | stressed) or an activity | feel anxiety) and stress, or our |
| | (being at a | response to them. In other |
| | frustrating meeting). | words, we can proactively build up |
| | Notice everything that's | our resilience against |
| | happening to | anxiety. And one effective way to |
| | you internally: your | do that is by practicing |
| | physical | mindfulness. |
| | sensations, thoughts, | Being mindful requires you to be |
| | feelings. Don't | aware of physical |
| | evaluate (for example, | sensations, perceptions, thoughts |
| | wondering | and imagery-even when |
| | whether you're thinking | your brain would rather be |
| | rationally), | worrying about the unpleasant |
| | just experience it. | situation itself. (And yes, it's much |
| | Afterwards, write | harder to practice living |
| | about what happened to | in the moment when you're |
| | you. Look at | stressed or angry than it is when |
| | each aspect of the | you're feeling at peace with the |
| | experience and see | world!) This exercise is |
| | if you can connect how | derived from Mindfulness-Based |
| | your physical | Stress Reduction |
| | sensations, thoughts, and | techniques. Numerous studies |
| | feelings | show that mindfulness |
| | interacted with each other. | practice, especially when done |
| | | regularly, can improve |
| | | general coping with stresses from |
| | | everyday life, as well as |
| | | under more extraordinary |
| | | conditions. |
| S-13 | Want to feel happier and | Research shows that the practice |
| | more connected with | of loving-kindness |
| | the world? (Who | meditation shifts people's day-to- |
| | wouldn't?) Take 10 | day emotions to be more |
| | minutes to try a | positive, and in so doing, it also |
| | simple exercise called | increases their overall |
| | loving-kindness | health and wellbeing. |
| | meditation, which | Barbara Fredrickson, Ph.D., Kenan |
| | can help you do | Distinguished Professor |
| | just that. Sitting quietly | of Psychology and Nemoscience at |
| | and comfortably, | the University of North |
| | breathe naturally and | Carolina at Chapel Hill has been |
| | pay attention to | investigating the various |
| | your breath. | benefits of positive emotions for |
| | Spend a few minutes | 25 years and has explored |
| | thinking loving, | the specific impact of loving- |
| | compassionate | kindness mediation for more |
| | thoughts about | than a decade. |
| | yourself. (Think | Experiments by Fredrickson and |
| | something like: | her team show that people |
| | “May I be happy.”) | who practice loving-kindness |
| | Then focus your loving | meditation show more self- |
| | thoughts on to | acceptance and enjoy more |
| | someone who is | positive connections with |
| | close to you, like | friends, family, and co-workers. |
| | your children. (“May | They also become more |
| | she be happy.”) | mindful, find more purpose in life, |
| | Next, transfer your | and are better able to |
| | thoughts to all | savor the good. They even report |
| | people. (“May they | fewer illness symptoms |
| | be happy.”) If | and show improved cardiac |
| | your mind wanders, | function. |
| | don't feel bad | |
| | about it-it's | |
| | natural!-just gently | |
| | bring your thoughts | |
| | back to the exercise. | |
| T-01 | Write down everything | Dr. Robert Emmons of the |
| | that's | University of California-Davis |
| | happened in the past week | has been at the forefront of |
| | that you | research into gratitude |
| | are grateful for-from | interventions. His research shows |
| | friends to | that people who kept |
| | experiences to personal | gratitude journals on a weekly |
| | accomplishments. | basis exercised more |
| | | regularly, reported fewer physical |
| | | symptoms, felt better |
| | | about their lives, and were more |
| | | optimistic about the |
| | | upcoming week compared to those |
| | | who recorded hassles or |
| | | neutral life events. |
| | | He also found another benefit |
| | | when it comes to attaining |
| | | goals: Participants who kept |
| | | gratitude lists were more likely |
| | | to have made progress toward |
| | | important personal goals |
| | | (academic, interpersonal and |
| | | health-based) over a two- |
| | | month period compared to |
| | | subjects in the other |
| | | experimental conditions. |
| T-02 | Keep a gratitude | One theory as to why gratitude |
| | journal about | could be linked with |
| | someone close to you. | improved well-being is that |
| | Write down | grateful people have stronger |
| | everything they do that | coping strategies than people who |
| | you're grateful | aren't grateful. |
| | for, then review it at the | In one study, gratitude was shown |
| | end of the | to relate to three broad |
| | week. Are there any | categories of coping. First, grateful |
| | patterns in what | people are more likely |
| | this person tends | to seek out support from their |
| | to do that you're | social networks when the |
| | grateful for? What | need arises. Second, grateful |
| | generalizations can | people face life's challenges |
| | you draw about who this | head-on. They're able to put their |
| | person is and | problems in perspective, |
| | what they mean to you? | plan solutions and learn from the |
| | | experience. And third, |
| | | grateful people are less likely to |
| | | run from or deny their |
| | | problems exist. These results may |
| | | explain why grateful |
| | | people are also less stressed. |
| | | Keeping a gratitude log can also |
| | | fortify your existing |
| | | relationships by producing feelings |
| | | of greater |
| | | connectedness. When you value |
| | | someone, you tend to treat |
| | | them better, which makes them |
| | | feel good and treat you |
| | | better. In several studies, |
| | | researchers have found that people |
| | | who are grateful towards particular |
| | | individuals in their lives |
| | | experience closer and “higher- |
| | | quality” relationships with |
| | | them, even if their gratitude is |
| | | never directly expressed. |
| T-03 | If you created gratitude | In one study, participants were |
| | log about | asked to write letter of |
| | someone close to you | gratitude to someone they wished |
| | during previous | to thank before reading |
| | activity, show it to the | the letter aloud to the recipient. |
| | person you | After the experiment, they |
| | wrote about. You can | reported immediate increases in |
| | email it, go | happiness and decreases in |
| | through your list on the | depressive symptoms, giving them |
| | phone, or | short-term boost in well-being. |
| | better yet-find an | In variant on the above |
| | opportunity to read | experiment, scientists recommend |
| | your list out loud, | keeping gratitude log for fixed |
| | in person. | period of time (for example, |
| | If you haven't started | one month) about person in your |
| | log, begin by | life for whom you're |
| | choosing someone in your | thankful. This exercise lets the |
| | inner circle | giver experience the |
| | and writing down | powerful happiness effects of |
| | everything they do | delivering the gratitude |
| | that you're grateful for | report, but keeps the exercise |
| | over one-week | fresh, since it can be |
| | period. Afterwards, | replicated with different |
| | review what | recipients each time. |
| | you've written and | |
| | look for any | |
| | patterns. What | |
| | generalizations can | |
| | you draw about who this | |
| | person is and | |
| | what they mean to | |
| | you? Finally, | |
| | deliver your log to the | |
| | person you | |
| | wrote about. | |
| | If you enjoyed this | |
| | exercise, you can | |
| | make this regular | |
| | practice, delivering | |
| | your “gratitude report” | |
| | periodically to | |
| | different people | |
| | in your life. | |
| T-04 | Jot down three things that | In study conducted by Drs. Martin |
| | happened | Seligman, Tracy Steen |
| | today or yesterday that | and Christopher Peterson, group of |
| | made you feel | people was asked to |
| | grateful. | practice this gratitude exercise |
| | It could be something | every day for one week. |
| | someone did for | Even though the exercise lasted |
| | you, something | just one week, at the one- |
| | you did for yourself, | month follow-up, participants were |
| | or just the simple fact that | happier and less |
| | the sun was | depressed than they had been at |
| | shining. Add photo, too! | baseline, and they stayed |
| | | that way at the three- and six- |
| | | month follow-ups. |
| | | This practice primes our mind for |
| | | gratitude, and helps |
| | | overcome the brain's natural |
| | | “negativity bias,” phenomenon |
| | | by which we are wired to give |
| | | more weight to negative |
| | | rather than positive experiences or |
| | | other kinds of information. |
| T-05 | Maybe you quit smoking | Studies show that affirming our |
| | or joined | sense of personal worth can |
| | gym. Maybe you | improve our ability to cope with |
| | controlled your | stress. Affirmations get us |
| | anger when you | to focus on higher values, rather |
| | were provoked. | than immediate impulses, |
| | Maybe you | and that boosts our ability to act in |
| | made someone laugh right | our best interests. |
| | when he/she needed | In fact, even brief, daily |
| | it most. | affirmations can have long-term |
| | This isn't the time for | benefits if they break our cycle of |
| | humility. What | negative rumination. So |
| | abilities come easily to | take few minutes and think of |
| | you? Which of | some things that are great |
| | your talents do others | about you! |
| | admire? Ask | Studies show that affirming our |
| | close friend, family | sense of personal worth can |
| | member or trusted | improve our ability to cope with |
| | co-worker which of your | stress. Affirmations get us |
| | skills they | to focus on higher values, rather |
| | find exceptional. | than immediate impulses, |
| | Take some time to indulge | and that boosts our ability to act in |
| | in being | our best interests. |
| | grateful for who you | In fact, even brief, daily |
| | are, whenever! | affirmations can have long-term |
| | | benefits if they break our cycle of |
| | | negative rumination. So |
| | | take few minutes and think of |
| | | some things that are great |
| | | about you! |
| T-06 | Think of someone in your | Numerous studies show that |
| | life whom | gratitude is one of the strongest |
| | you've never properly | predictors of positive mental |
| | thanked. Your | health. People who are grateful |
| | gratitude could be for | are more satisfied with their |
| | particular | relationships with friends and |
| | instance when they | family. They're happier, less |
| | helped you, or it | depressed and less stressed. |
| | could be very general-or | They feel more in control of their |
| | both. It can | lives, have higher self- |
| | be someone you | esteem and cope better with stress. |
| | see all the time, or | In one study, ~-participants were |
| | someone you haven't seen | asked to write letter of |
| | in while. | gratitude to someone they wished |
| | Now, write letter | to thank before reading |
| | detailing your | the letter aloud to the recipient. |
| | gratitude to that | After the experiment, they |
| | person. It should be at | reported immediate increases in |
| | least one page, ideally | happiness and decreases in |
| | longer. Reflect, | depressive symptoms. And |
| | with great detail, | research by Sonja Lyubomirsky, |
| | on why you feel | Rene Dickerhoof, and Julia Boehm |
| | thankful towards them. | has shown that you can |
| | | get substantial boosts in happiness |
| | | from writing gratitude |
| | | note even if you don't deliver |
| | | your letter. |
| T-07 | If you wrote gratitude | Numerous studies show that |
| | letter to | gratitude is one of the strongest |
| | someone you're grateful | predictors of positive mental |
| | to during an | health. People who are grateful |
| | earlier activity, deliver | are more satisfied with their |
| | it to them- | relationships with friends and |
| | ideally in person, but | family. They're happier, less |
| | you could read it | depressed and less stressed. |
| | to them over the phone or | They feel more in control of their |
| | send an | lives, have higher self- |
| | email if the recipient is | esteem and cope better with stress. |
| | halfway across | In one study, participants were |
| | the globe. | asked to write letter of |
| | (Haven't written gratitude | gratitude to someone they wished |
| | letter yet? | to thank before reading |
| | First think of someone in | the letter aloud to the recipient. |
| | your life | After the experiment, they |
| | you've never properly | reported immediate increases in |
| | thanked, then | happiness and decreases in |
| | express your | depressive symptoms. |
| | gratitude in note.) | |
| | If you can, capture the | |
| | note-or the | |
| | moment itself!-in photo. | |
| T-08 | Think of something or | Numerous studies show that |
| | someone in | gratitude is one of the strongest |
| | your life for whom you're | predictors of positive mental |
| | grateful. | health. People who are grateful |
| | Discuss your feelings with | are more satisfied with their |
| | someone | relationships with friends and |
| | else who shares that same | family. They're happier, less |
| | gratitude. It | depressed and less stressed. |
| | may help if you have | They feel more in control of their |
| | written gratitude | lives, have higher self- |
| | letter about the | esteem and cope better with stress. |
| | target of the | This exercise is variant of several |
| | discussion, so you can | interventions that involve |
| | share it with | either keeping gratitude log or |
| | the other person | writing gratitude letter. Both |
| | to spark the discussion. | Sonja Lyubormirsky and Martin |
| | | Seligman have conducted |
| | | studies with participants asked to |
| | | write letter of gratitude to |
| | | someone they wished to thank |
| | | (and in some cases, |
| | | delivering it to that person). In |
| | | both cases, they reported |
| | | immediate increases in happiness |
| | | and decreases in |
| | | depressive symptoms. Scientists |
| | | also know that people |
| | | enjoy things more when they |
| | | do it with other people, and |
| | | that spending quality time with |
| | | others is highly effective |
| | | mood-booster. What could be |
| | | more powerful than bonding |
| | | with friend over your |
| | | shared gratitude? |
| T-09 | Think of something, great | Numerous studies show that |
| | or small, | gratitude is one of the strongest |
| | that you feel grateful for | predictors of positive mental |
| | and describe | health. People who are grateful |
| | it in few words. | are more satisfied with their |
| | Add photo too! | relationships with friends and |
| | | family. They're happier, less |
| | | depressed and less stressed. |
| | | They feel more in control of their |
| | | lives, have higher self- |
| | | esteem and cope better with stress. |
| | | When the going gets tough, |
| | | grateful people learn from the |
| | | experience. They don't avoid the |
| | | problem, deny that |
| | | anything's wrong, or blame |
| | | themselves. That's probably |
| | | why grateful people also sleep |
| | | better! |
| | | In 2005 study, some participants |
| | | were asked to transcribe, |
| | | every night for one week, three |
| | | positive events that |
| | | happened during their day, as well |
| | | as the causes of these |
| | | events. Compared to participants in |
| | | the control group, those |
| | | who reflected on three positive |
| | | events experienced more |
| | | happiness. |

### Specific to Migraine Digital Therapeutic

The first intervention in the migraine digital therapeutic provides information about symptoms and side effects, MDD and/or GAD symptoms, and how those symptoms are related to the treatment the patient needs and will complete. The therapeutic modality label for this type of intervention is psychoeducation. Later in treatment, an intervention that provides information about how specific therapeutic activities (mindfulness, for example) help GAD and/or MDD may be included. It also falls under psychoeducation.

When personalization of the treatment for key interest areas or disease-specific therapy is sought, it is important that the therapeutic modality remains the same. One cognitive intervention may be replaced with another cognitive intervention, but replacing cognitive intervention with psychoeducation intervention would be avoided. The complete order of therapeutic modalities is referred to as the indication treatment sequence. It is the order of all interventions over the multi-week treatment that is an important consideration in designing any digital therapeutic for maximum efficacy and adherence levels. Following sequence also impacts the treatment's safety and efficacy, these factors have been considered in designing the multi-week treatment.

In treatment relevant to MDD and/or GAD, targeting and preventing worry behaviors such as frequent telephone calls to loved ones, refusal to read obituaries, or cleaning one's house daily in case someone drops by, may be monitored. The therapy helps the patient focus on the specific behavior that is relevant to that patient. Flexibility is also essential in therapy when patient does not enjoy or cannot complete specific intervention. For example, progressive muscle relaxation is standard intervention in many MDD and/or GAD treatments. Briefly, the patient tenses then relax muscles throughout their body to achieve more relaxed physiological state. However, some patients experience paradoxical “relaxation-induced anxiety” that predicts poor outcomes. For patients who experience such anxiety, it is appropriate to find an alternative method for providing relaxation.

It is imperative to personalize CBT-based treatments for people suffering from chronic medical conditions like migraine. While the active ingredients are the same for people living with vs. without these conditions, some language, examples, and recommendations can provoke negative reactions in people with chronic medical conditions. People who require the use of walker or wheelchair, for example, might not appreciate walking meditation. People who have recently had an organ transplant may be unable to travel far from their medical support team, so examples involving travel might need to be reworked. These kinds of changes do not fundamentally alter the treatment but provide more supportive treatment experience. It is intended to use migraine digital therapeutic to provide personalized treatment to specific populations in which MDD and/or GAD is prevalent, such as people diagnosed with migraine.

In migraine sufferers and especially persons suffering chronic migraine, untreated anxiety and depression can contribute to poor control over the condition and exacerbate physical symptoms. Treating anxiety and depression in people living with migraine may or may not directly impact their physical health, but by reducing negative emotions and unhelpful behavioral patterns, patients can manage their conditions more effectively. Management of the condition also includes management of any side effects resulting from medications the patient is taking to treat their condition. For example, a CGRP receptor antagonist may have intestinal side-effects that can be addressed and specifically managed with particular activities and tracks.

CBT and related behavioral therapies can reduce anxiety and depression in people with chronic medical conditions. However, some personalization is required to ensure the treatment resonates with the patients. Such personalization is not consistently achievable in traditional, face to face therapy. Economics, logistics, training and organization are merely the most readily apparent reasons for this. Given the number of different factors involved per different chronic condition, it is simply not possible to match properly trained therapists with each patient suffering chronic condition. Clinicians with advanced training in CBT and other important therapies would need to complete supplemental training in the specific population to ensure they are fully prepared to provide such treatment at optimum levels. It is simply not possible for given clinician to have training across even significant percentage of all chronic conditions, symptomologies, side effect profiles and other potential therapeutic areas. A digital therapeutic is, in contrast, ideally suited to provide therapy directed to essentially any number of conditions, symptoms, side effects, etc.; therapy not only highly personalized to an individual patient but also personalized to one or more conditions impacting the mental health of the individual patient. The above-mentioned optimum level of treatment based upon proper training, ready access to all tools updated to the time of treatment, integration of all available research/trials, selecting appropriate protocols, etc., is not possible for even the best human therapist. For digital therapeutic, however, such personalized treatment is possibility. The ability to scale digital therapeutics is even more important for those living with chronic medical conditions than those who are not.

The present migraine digital therapeutic provides standardized series of interventions. The product may offer personalized experience based on the patient's key area-of-interest (AOI) as an overlay on the standardized activity series. Some examples of AOIs are family, career, and physical wellness. The patient can choose an AOI or complete the treatment without AOI personalization. The different options may have the same indication treatment sequence or modified one. The therapeutic modality of each intervention will be the same for each of the treatment options or may be different. For identical therapeutic modalities it is expected that the efficacy of each option is likely to be identical.

Table 3 below shows an example of three activities from the migraine digital therapeutic. Based on the Indication Treatment Sequence created for the product, the first three interventions may be selected from the following therapeutic modalities: Mindfulness, Cognitive, Acceptance. The Career option's specific interventions are focus on your breath for minutes, reframe negative thought—career focus, and identify fact that is hard for you to accept—career focus. The family option's specific interventions are Focus on your breath for minutes, reframe negative thought—family focus, radical acceptance—family focus. The same principle applies across the full migraine digital therapeutic indication treatment sequence.

TABLE 3

| | | | Therapeutic |
|---|---|---|---|
| Intervention | Area of Interest: Career | Area of Interest: Family | Modality |
| | Focus on your breath for | Focus on your breath for minutes | MINDFULNESS |
| | minutes | | |
| | Choose negative thought | Choose negative thought related to your family. | COGNITIVE |
| | related to your career. Put | Put the thought on trial. Imagine you are | |
| | the thought on trial. Imagine | lawyer. What is the evidence for and against | |
| | you are lawyer. What is the | this thought? | |
| | evidence for and against this | | |
| | thought? | | |
| | Identify something about | Think of something about one member of your | ACCEPTANCE |
| | your job that you wish were | family that really drives you crazy. Now take | |
| | different. Describe how it is | deep breath and focus on letting go of your | |
| | right now in detail. Try to | efforts to change this. Work toward accepting at | |
| | identify what makes it hard | deep level that this person is not going to | |
| | for you to accept that this is | change this part of who they are. Think about | |
| | true about your job. | how your own actions can change if you give | |
| | | up the idea that this person will change. | |

Personalizing treatment for people living with chronic medical conditions like migraine can fill significant gap in mental health treatment as well as in the treatment of concomitant migraine symptoms or treatment side effects. As stated previously, these treatments are effective but require additional knowledge and training in order to be delivered efficiently, properly and competently. It is unrealistic to expect busy practitioners to gain needed expertise in each subgroup of people living with chronic medical conditions. Therefore, it is particularly valuable to have digital therapeutic that can be personalized and delivered efficiently at scale to everyone who needs it.

The methods for personalizing migraine digital therapeutic for people living with chronic medical conditions are similar to the methods for adapting it to an area of interest. Changes may be made to ensure the interventions are appropriate and impactful for given subpopulation, changes to the indication treatment sequence may be assessed but may be unnecessary. The interventions included in versions of migraine digital therapeutic that have been personalized for people living with chronic medical conditions will have interventions that reflect the same modalities that may be provided in the same or different order as in the standard version.

Table 4 below shows the three options for personalization for an intervention. The need to change interventions are expected to be highly variable depending upon the condition, symptoms, side effects and related concerns that will be fact dependent from condition to condition. Some minimal changes are shown in examples below. These include modifications to wording, such as different example to illustrate an idea, or physical exercise modification to allow for common physical limitation. A small number of interventions may need to be replaced with different intervention from the same therapeutic modality. Example below illustrates how one intervention from the “mindfulness” modality is exchanged for another. By adding these subtle personalization elements, more supportive treatment may be developed that will encourage people with specific chronic conditions to engage with the treatment more than they would without such personalization.

Table 4 shows interventions that may appear in standard MDD and/or GAD treatment compared to migraine-oriented model. The wording has been modified to make it appropriate for someone with migraine. Intervention is identical for the two treatment models. Intervention has very small change, but is nearly identical between the two treatment models. Intervention is an entirely different activity for the standard MDD and/or GAD treatment model vs the migraine-oriented MDD and/or GAD treatment model, but it comes from the same therapeutic or behavioral modality.

TABLE 4

| | | | Therapeutic |
|---|---|---|---|
| Example | No chronic condition | Chronic condition: Migraine | Modality |
| (no change) | Keep daily diary of your worry content, | Keep daily diary of your worry | DAILY |
| | anxiety level, and whether it’s current | content, anxiety level, and whether | MONITORING |
| | or potential problem. | it’s current or potential problem. | |
| (minimal | Worry is part of life for everyone, but | Worry is part of life for everyone, | PSYCHO- |
| change) | for people with GAD it can get out of | but for people with GAD it can get | EDUCATION |
| | control. The physiological arousal that | out of control. The physiological | |
| | comes with excessive worry can make | arousal that comes with excessive | |
| | it hard to concentrate, interfere with | worry can make it hard to | |
| | your sleep, and make you irritable. | concentrate, interfere with your | |
| | | sleep, and make you irritable. For | |
| | | people with migraine, these | |
| | | problems can make it harder to | |
| | | follow your treatment plan and stay | |
| | | healthy. | |
| (different | One good way to manage worry is to | One healthy way to manage worry | MINDFULNESS |
| intervention, | refocus your attention from worry | is to engage in something | |
| but same | about the future to vigorous activity, | enjoyable instead. This will help | |
| therapeutic | such as running or jumping jacks. | get you out of your negative | |
| modality) | Choose level that is difficult for you, | thought spiral and focused on the | |
| | but that you are confident you can | present. Choose something you | |
| | complete. If you have heart rate | enjoy, like cooking, playing | |
| | monitor, see if you can get your heart | musical instrument, or doing | |
| | rate up to somewhere between 85 and | crossword puzzle. Try to throw | |
| | 145 bpm for about minutes. Slowly | yourself into it completely and take | |
| | cool down and see how you feel. | break from worry. | |

Modifying a generally useful and potentially FDA cleared product for a specific patient population has several benefits. There are often small changes that need to be made to ensure that the more specific population's developed skill sets resonate within the app.

An example of small changes for migraine is that people with migraines, especially toward more severe end of spectrum, might worry about the onset of their next migraine. While many worrying thoughts are unlikely to come true, the question of when a migraine will occur in a person with chronic migraine is both valid and reasonable. The therapeutic product could address this and encourage the patient to focus instead on considering how they will cope when the migraine arrives.

It is very useful to include additional information helpful to people with migraine, such information may be integrated with various portions of the app. For example, some conditions are treated with reasonable number of medication types or have particular symptoms. Further, side-effects of medication types are also a known issue that may be planned for in the app. Migraine is typically dealt with using one or more of about 6-8 medications or other treatments of variable efficacy, patient to patient. The efficacy of these medications also varies from symptom to symptom. The chatbot integrated with the app, e.g., Anna, might therefore ask about these medications, symptoms and side-effects and reference them in future dialogues.

Another potentially useful feature is to connect patients suffering the same condition to each other through our product's community features. Such feature may even drill down to particular symptoms and side-effects impacting a group of patients and connect them with reference thereto. These different groups might require different guidelines for discussions. For example, people with migraine who are recovering from surgery might want a community where details of triggers, aura, etc. are discussed or, potentially, not discussed. Similarly, a community discussing the efficacy of treatments might be interesting to a patient prior to deciding on whether to try that particular treatment.

A migraine digital therapeutic app may be used in the home as prescription device, under the management of licensed healthcare provider, for the treatment of migraine. The migraine app presented here has been developed under design controls developed as part of the Program's Quality System.

The development of the migraine digital therapeutic app has operated under applicable FDA regulations, FDA Guidance and consensus standards for software as medical device. This includes conformance with the following: 21 CFR 820.30 Design Controls, Guidance for the Content of Premarket Submissions for Software Contained in Medical Devices, Content of Premarket Submissions for Management of Cybersecurity in Medical Devices, Software as Medical Device (SaMD): Clinical Evaluation, ISO 14971, and IEC 62304. Also, each release of the device undergoes software testing in accordance with FDA's Guidance, “General Principles of Software Validation” to further ensure that the software performs as intended.

Key design and development elements of a quality system include configuration management plan, software requirements specification, software development plan, software verification & validation plan, software risk analysis, and software defect tracking. Functional verification testing ensure the software performs per specification prior to clinical validation. Development through the design control and risk management processes provide the results and data necessary to demonstrate safety, effectiveness and overall quality of the migraine digital therapeutic app. Iterative bench testing and bug fixes are captured and documented by the developers. More formal verification and validation testing will be conducted subsequent to release. Verification and validation tests provide traceability back to design documents and the IEC 62304 requirements. In addition to software testing, the developer conducts reliability testing and human factors testing. Usability and risk of user error (intended and unintended miscue) may be studied though human factors engineering studies. Bugs and defects identified at this stage are captured using a tracking system. During the validation stage fixes would be approved in formal change order (CO) protocols.

### Migraine

Migraine is a complex, common neurological condition characterized by severe, episodic attacks of headache and associated features such as nausea, vomiting, sensitivity to light, sound or movement. In some patients, the headache is preceded or accompanied by an aura. The headache pain may be severe and sometimes occurs on one side of the brain. This is called unilateral migraine. Migraine in about 15% of patients is “side-locked” in that they only get migraine headache on one side. In North America and Western Europe, the overall prevalence of migraine patients is 11% of the general population, i.e., 6% in males and 15-18% in females. The median frequency of migraine attack in an individual is one or two per month, though the deviation from this mean is substantial. There is a strong genetic component to migraine.

Chronic migraine is when a migraine occurs 15 or more days per month. Symptoms in chronic migraine often change frequently as may the severity of the pain. Primarily due to the high frequency of chronic migraine, it has a particularly debilitating impact on the patient's quality of life and has the potential to be a primary feature of the patient's life. Sufferers of chronic migraine have high incidence of depression, anxiety, employment issues and lower socioeconomic status than the general public. Chronic migraine affects about 2% of the general population.

A migraine is much more than a bad headache. Migraine attacks are often disruptive to daily life. The throbbing pain is often debilitating and its debilitating impact typically lasts several hours but may last days. Onset of a migraine attack may be associated with triggers that include movement, light, sound and many others. A migraine may involve one or more symptoms like neurological pain, tiredness, nausea, visual disturbances, numbness and tingling, irritability, difficulty speaking, temporary loss of vision and many more. Migraine is a common neurological disease having a most prevalent symptom of a throbbing, pulsing headache on one side of head. Migraine symptoms typically worsen with physical activity, lights, sounds or smells.

An aura is a group of sensory, motor and speech symptoms that usually act like warning signals that a migraine headache is approaching. Sometimes misconstrued as a seizure or stroke, aura typically happens before the headache pain, but can sometimes appear during or even after the migraine episode. An aura can last from 10 to 60 minutes and occur in about 15% to 20% of people who experience migraines.

Aura symptoms include seeing bright flashing dots, sparkles, or lights, blind spots in vision, numb or tingling skin, speech changes, ringing in ears (tinnitus), temporary vision loss, seeing wavy or jagged lines, changes in smell or taste, and a “funny” feeling.

There are several types of migraines and the same type may go by different names. Migraine with aura is also referred to as a complicated migraine, occurring in about 15% to 20% of people with migraine headaches. Migraine without aura is also referred to as common migraine. This type of migraine headache strikes without the warning, though the symptoms are the same, other than lack of aura symptoms. Migraine without head pain is referred to as silent migraine or acephalgic migraine and includes the aura symptom but not the headache that typically follows. Hemiplegic migraine involves temporary paralysis (hemiplegia) or neurological or sensory changes on one side of the body. Onset of hemiplegic migraine headache may be associated with temporary numbness, extreme weakness on one side of the body, a tingling sensation, a loss of sensation and dizziness or vision changes. Sometimes it includes headache and sometimes it does not. Retinal migraine is sometime referred to as ocular migraine and has symptoms including temporary, partial or complete loss of vision in one eye, along with a dull ache behind that eye that may spread. Vision loss may last a minute or as long as months.

Migraine with brainstem aura is migraine accompanied by vertigo, slurred speech, double vision or loss of balance, which symptoms occur before the headache. The headache pain may affect the back of the head. Migraine with brainstem aura symptoms usually occur suddenly and can be associated with the inability to speak properly, ringing in the ears and vomiting. Status migrainosus is a rare and severe type of migraine that can last longer than 72 hours. The headache pain and nausea can be extremely bad. Certain medications, or medication withdrawal, can cause this type of migraine.

The four stages of typical migraine are, in chronological order, the prodrome (premonitory), aura, headache and postdrome. About 30% of people experience symptoms before their headache starts. Prodrome may last a few hours or a few days and is sometimes referred to as the “preheadache” or “premonitory” phase. The aura phase can last as long as 60 minutes or as little as five. Most people do not experience an aura, and some have both the aura and the headache at the same time. Headache lasts about 4 hours to 72 hours. Although sometimes mild, the headache pain is typically intense, starting on one side of the head and spreading to the other side. Postdrome follows the headache and lasts for a day or two. It has been called a migraine “hangover” and 80% of those who have migraines experience it.

The cause(s) of migraine remain little understood. Changes is the brainstem and the interaction of the brainstem with the trigeminal nerve, a major pain pathway, may be involved. Imbalances in brain chemicals, e.g., serotonin, may also be a factor. Serotonin helps regulate pain in the nervous system and its role has been a focus of migraine research. Other neurotransmitters have been receiving attention with regard to migraine research, including calcitonin gene related peptide (CGRP), discussed further hereinbelow.

The primary risk factors for migraine include genetics, gender, stress level and smoking. About 80% of people who get migraine headaches have a first-degree relative with the disease. Migraine headaches are two to three times more prevalent in women than in men, this is especially true for women between the ages of 15 and 55. These two facts, among others, contribute to the strong evidence that female hormones influence risk factors.

Hormonal changes, stress and smoking exist on the border between causes/risk factors for migraine and migraine triggers. Fluctuations in estrogen seem to trigger migraines, as do higher stress levels, smoking, caffeine, sensory stimuli such as strong lights, loud sounds and strong smells, changes in sleep patterns, physical exertion, weather changes, medications, some foods and food additives.

One outcome from the lack of understanding of causation in migraine is difficulty in treating the disease. The first option regarding migraine is prevention. That is, reducing the frequency and/or severity of migraine episodes. Success has been achieved in this regard connected with a number of the triggers mentioned previously. Reduction in stress, smoking, exposure to strong light, etc., are all actions taken by migraine patients to reduce and/or limit the severity of migraines. The efficacy of addressing the triggers of migraine has a very high level of variability among patients. Perhaps more importantly, the length of time such trigger avoidance is effective also has a high level of variability from patient to patient. That is, elimination of some triggers will have zero efficacy for some patients and long-term efficacy for others, with the majority falling somewhere in between.

Drugs for migraine headaches can relieve the pain and other symptoms of migraine and/or may help prevent future migraine episodes. Abortive treatments are those that seek to reduce or eliminate a migraine once it starts or once the patient feels that a migraine is approaching. Abortive medications are particularly useful in persons with prevalent nausea/vomiting symptoms. Preventive treatments seek to lessen the frequency and severity of migraine attacks and are typically taken on a set schedule, e.g., daily or weekly. Prevention is considered if migraines occur frequently, i.e., more than once per week, or if migraine symptoms are severe. Abortive treatments include triptans and ditans, which specifically target serotonin. Such drugs include almotriptan, eletriptan, frovatriptan, naratriptan, rizatriptan, sumatriptan and zolmitriptan.

Over the counter pain medications and combination pain medications have been used for migraine essentially since their introduction. The most used drugs include those containing ibuprofen, aspirin, acetaminophen, caffeine, isometheptene and dichloralphenazone. Drugs containing mixtures of these compounds are popular, including Excedrin® Migraine which contains aspirin, acetaminophen and caffeine. Ergot alkaloids, including dihydroergotamine and ergotamine, are used to treat migraine often in combination with caffeine and other compounds.

Antagonists to calcitonin gene related peptides (CGRP), discussed further hereinbelow, are also used as abortive treatments for migraine. Abortive treatments for migraine related nausea include chlorpromazine (Thorazine®), droperidol, metoclopramide and prochlorperazine. Drugs for headache pain but not specific to migraine include analgesics, narcotics and barbiturates though these drugs are less ideal due to potential to be habit forming.

Some abortive treatments, especially when used by chronic migraine patients, can lead to worsening of chronic migraine. Overuse of such treatments often results in a secondary headache called a medication overuse headache. Further, such treatments often have well known cardiovascular and gastrointestinal side-effects, e.g., chronic use of nonsteroidal anti-inflammatory drugs (NSAIDs) increases risk of peptic ulcer, renal failure, stroke and myocardial infarction.

Preventative treatment medications are more likely to be administered as the frequency or severity of migraine symptoms increase. Some high blood pressure medications have been prescribed as preventative treatments, including beta-blockers such as propanolol, timolol and metoprolol as well as calcium channel blockers such as verapamil. Antidepressant medications such as amitriptylin and nortriptyoline have been utilized. Antiseizure medications like gabapentin, topiramate and valproic acid have been prescribed. Again, calcitonin gene related peptides are discussed further hereinbelow and have also been prescribed as preventative treatment therapeutics for migraine. Injectable botulinum toxin (Botox®) prevents the release of the neurotransmitter acetylcholine from axons near the neuromuscular junction, causing a type of paralysis and is an FDA approved treatment for chronic migraine headache.

Several medical devices are also available for treatment of migraine headache. Some of these devices are based on the premise that migraine causation or symptoms of migraine are related to neuronal activity in the brain and that modulation of this neuronal activity will have an effect on migraine and/or its symptoms. Cefaly® is a small headband device that sends electrical pulses through the forehead to stimulate a nerve linked with migraines. Cefaly® is an electronic transcutaneous nerve stimulation (“e-TNS”) device available over the counter and is approved by the FDA for migraine treatment and prevention. Single Pulse transcranial magnetic stimulators (“sTMS”) are based on the theory that aura in migraine results from a wave of unusual electrical activity called cortical spreading depression. A device that emits relatively strong pulse(s) of magnetic energy may disrupt this wave and thus prevent the onset of aura. One sTMS device is the eNeura sTMS Mini® which is a small device held to the back of the head by the user which emits a short magnetic pulse. Spring TMS® is similar to eNeura sTMS Mini®. Both of these TMS devices are FDA approved. A noninvasive vagus nerve stimulator (nVS) is a hand-held portable device placed over the vagus nerve in the neck that releases a mild electrical stimulation to relieve pain. It appears that several nVS devices have been approved by the FDA for use in treating migraine.

Many migraine patients are encouraged to keep a migraine journal that may assist the patient and their healthcare provider with the diagnosis and the identification of triggers. A highly detailed and frequently updated journal may be a useful tool but the ability of a patient to keep such a journal, even for a short span of time, is highly variable. Things tracked in such a journal include date and time of when the migraine/prodrome started, whether symptoms preceded the head pain, time periods of the four stages, levels of pain, unilateral/bilateral spread, other symptoms accompanying headache, etc. Patterns can be a very helpful tool, including anticipation of what will happen in the future. Diary entries as to how many hours of sleep per night, stress level, weather, food/water/alcohol intake, medications taken, etc., are all helpful things in such a diary, permitting insight into triggers and other migraine factors. Similarly, medications or other treatments attempted for a given migraine and their efficacy is very useful data to track. A number of smartphone apps have sought to take the place of things like a migraine journal with mixed success.

### CGRP Drugs in Migraine

The calcitonin superfamily of peptides includes at least five known members: calcitonin, amylin, adrenomedullin, and two calcitonin gene-related peptides (“CGRP”), CGRP1 (also known as ctCGRP, or CGRP) and CGRP2 (also known as (3CGRP).

CGRP and physiological changes linked thereto have been shown to be present in migraine. CGRP is a 37 amino acid vasoactive neuropeptide expressed in both the central and peripheral nervous systems and has been shown to be a potent vasodilator in the periphery, where CGRP-containing neurons are closely associated with blood vessels. CGRP-mediated vasodilatation is associated with neurogenic inflammation, as part of a cascade of events that results in extravasation, i.e., leakage, of plasma and vasodilation of the microvasculature.

Amylin (“Amy”) has specific binding sites in the central nervous system (“CNS”) and is thought to regulate gastric emptying and have a role in carbohydrate metabolism. Adrenomedullin is a potent vasodilator and has specific receptors on astrocytes and its messenger RNA is upregulated in CNS tissues that are subject to ischemia.

Calcitonin is involved in the control of bone metabolism and is also active in the CNS. The biological activities of CGRP include the regulation of neuromuscular junctions, of antigen presentation within the immune system, of vascular tone and of sensory neurotransmission. Three calcitonin receptor stimulating peptides (CRSPs) have also been identified in a number of mammalian species; the CRSPs may form a new subfamily in the CGRP family.

Further to CGRP in particular, the peptide chain of 37 amino acids is produced primarily in peripheral and central neurons. Although technically a hormone, many attributes and functions of CGRP1 are similar to those of a neurotransmitter. In the spinal cord, the function and expression of CGRP1 differs relative to its location of synthesis. Besides its vasoactive functions, CGRP1 can function in transmission of nociception, may contribute to regeneration of nervous tissue, may be linked to pain transmission, is thought to play a role in cardiovascular homeostasis, acts as a chronotype in the heart by increasing heart rate, is known to modulate the autonomic nervous system, has moderate effects on calcium homeostasis and plays a role in ingestion.

The receptor for CGRP1 has more than one part. One part of the receptor is a G protein-coupled receptor known as the calcitonin receptor-like receptor (“CRLR”). The other part is also a transmembrane protein, this one is called a receptor activity-modifying protein (“RAMP”). When RAMP1 interacts with CRLR a CGRP receptor results whereas when a RAMP3 interacts with CRLR a dual CGRP and adrenomedullin receptor results. This results from the RAMP family of polypeptides acting as receptor modulators that determine the ligand specificity of receptors for the calcitonin peptide family members. Unless associated with a RAMP, CRLR is not known to bind any endogenous ligand.

CGRP is a potent vasodilator that has been implicated in the pathology of a number of vasomotor symptoms, such as all forms of vascular headache, including migraines (with or without aura) and cluster headache. Migraine pathophysiology involves the activation of the trigeminal ganglia, where CGRP is localized, and CGRP levels significantly increase during a migraine attack. This in turn, promotes cranial blood vessel dilation and neurogenic inflammation and sensitization. Further, the serum levels of CGRP in the external jugular vein are elevated in patients during migraine headache. Intravenous administration of human ci-CGRP induced headache and migraine in patients suffering from migraine without aura, supporting the view that CGRP has a causative role in migraine.

Possible CGRP involvement in migraine has been the basis for the development and testing of a number of compounds having some impact on CGRP. Triptans are a family of drugs used as abortive migraine medications; about a half-dozen triptans have been approved by the U.S. FDA. The agonist effects of triptans on serotonin receptors in blood vessels and nerve endings result in the inhibition of CGRP. Several proposed compounds, e.g., BIBN4096BS, antagonize the CGRP receptor, thus inhibiting CGRP. A potent small-molecule CGRP antagonist, telcagepant (MK-0974), has been shown to relieve moderate-to-severe migraine attacks, including migraine pain and migraine-associated symptoms.

Erenumab-aooe (AIMOVIG®) is a monoclonal antibody that binds with high affinity to the CGRP receptor, antagonizing the receptor's function. Erenumab-aooe was first in class of monoclonal antibody therapies for migraine when allowed by the FDA in May 2018.

Fremanezumab (AJOVY®) and galcanezumab (EMGALITY®) are both monoclonal antibody based drugs that also antagonizes the CGRP receptor and were approved by the FDA subsequent to erenumab-aooe.

Erenumab-aooe (“erenumab”) is a human immunoglobulin G2 (IgG2) monoclonal antibody that has high affinity binding to CGRP receptor. Erenumab-aooe is produced using recombinant DNA technology in Chinese hamster ovary cells. It is composed of 2 heavy chains, each containing 456 amino acids, and 2 light chains each containing 216 amino acids. Erenumab-aooe is supplied as a sterile, preservative-free, solution for subcutaneous injection. Each 1 mL prefilled single-dose injector, whether autoinjector or glass syringe, contains 70 mg erenumab-aooe, 1.5 mg acetate, 0.10 mg polysorbate 80 and 73 mg sucrose. Recommended dosage is 70 mg once monthly with some patients benefitting from a dosage of 140 mg once monthly.

In a randomized, multi-center, 3-month, placebo-controlled, double-blind study evaluating erenumab as a preventive treatment of chronic migraine, 667 patients with a history of chronic migraine with or without aura were randomized such that 191 received 70 mg erenumab, 190 received 140 mg erenumab and 286 received placebo by subcutaneous injections once monthly for 3 months. Patients were allowed to use acute headache treatments including migraine-specific medications, i.e., triptans, ergotamine derivatives) and NSAIDs during the study. The mean migraine frequency at baseline was approximately 18 migraine days per month and was similar across treatment groups. At both the 70 mg and 140 mg monthly dosages, the change from baseline in migraine days per month was −6.6 days. Further, 39.9% of the 70 mg dosage group and 41.2% of the 140 mg dosage group cut their monthly migraine days by at least one-half.

Blocking intestinal calcitonin gene-related peptide (CGRP) with a CGRP antagonist such as erenumab, a medicine used for migraine prevention, may lead to constipation, which can be severe in some patients. This side effect is the result of the gastrointestinal (digestive) tract containing CGRP proteins. Some studies suggest that CGRP may play an important role in maintaining the movement of the bowels. Most people who develop constipation with erenumab do so after the first injection, but it may also occur later. In the clinical studies involving erenumab, constipation was one of the most common adverse reactions reported, occurring in about 3 out of 100 patients. Higher monthly dosing of erenumab correlates with higher incidence of constipation.

In some people, the constipation with erenumab is severe enough that constipation related complications result. Hospitalization or surgery may be needed in some cases. Thus, making patients aware of the issue, monitoring patients for constipation and dealing with the issue in a timely and effective manner are all important when constipation arises as a side effect

### Additional Embodiments

The foregoing disclosure of a digital therapeutic app for the treatment of migraine is not intended to be limiting.

A component of the invention lies in acquiring ongoing and real time input data from the user and performing analysis to respond more empathetically and more emotionally and more in context. However, the extent of the analytic capability by the AI is not limited to simply detecting the “tone” or identifying certain “topics.” For example, the artificially intelligent computing system can analyze input data to ascertain whether the user is answering the question truthfully, whether the user is only providing a partial answer to an inquiry, whether the user is engaged with enthusiasm or lack of enthusiasm, the extent to which the user is interested in the activity being performed, and whether the user prefers certain types of activities over other types of activities. In addition, when the user's response is analyzed, the computing system may detect not only topics, but also entities, and what the user's sentiment is toward these entities. Any of these analyses may be performed in addition to, or in conjunction with, the above-described analyses to develop a conversation that is emotionally specific.

In accordance with the present invention, the techniques as disclosed herein for the computing system to utilize AI in demonstrating empathy and providing more in context response goes far beyond merely automating what may occur in a typical current-day therapy session. One most notable advantage of the present computing system is its capability of providing a “super human” therapy or coaching session. A human therapist/coach bases his or her treatment based on familiarity with X number of patients. In contrast, the computing system of the present invention implements mirroring and other data-driven methods based on data collected from millions of users. For example, the computing system of the present invention knows how people tend to respond to a certain question much better than any single human therapist. Moreover, the computing system in accordance with the present invention can choose from a very large number of prompts, or generate new prompts from using natural language generation tools, some of which may include scientific facts, quotes, etc. in a way that significantly exceeds the capacity of a single human therapist. For example, if a user is into Indonesian movies from the 1950s, the computing system can find and/or generate a prompt weaving that into the conversation. No human therapist can personally relate to all topics that interest millions of people.

In accordance with the present invention, the English language is not intended to limit application or scope of any of the foregoing aspects of the present invention. For example, the classifier may be trained in multiple languages and one or more of the known techniques employed may work equally in different languages. In some embodiments, the artificial intelligence of the computing system may also learn cultural uniqueness in regards to tone, or in regards to conveyance of empathy in general, and adapt accordingly.

As herein used, a computer readable storage medium is not to be construed as being transitory signals per se, such as radio waves or other freely propagating electromagnetic waves, electromagnetic waves propagating through a waveguide or other transmission media, or electrical signals transmitted through a wire. The computer readable storage medium may be, but is not limited to, e.g., a magnetic storage device, an electronic storage device, an optical storage device, a semiconductor storage device, an electromagnetic storage device, or any suitable combination of the foregoing, and can be a tangible device that can retain and store instructions for use by an instruction execution device. The following is a list of more specific examples of the computer readable storage medium, but is not exhaustive: punch-cards, raised structures in a groove, or other mechanically encoded device having instructions recorded thereon, an erasable programmable read-only memory, a static random access memory, a portable compact disc read-only memory, a digital versatile disk, a portable computer diskette, a hard disk, a random access memory, a read-only memory, a memory stick, a floppy disk, and any suitable combination of the foregoing.

The operations of the present invention may be carried out by program instructions which may be machine instructions, machine dependent instructions, microcode, assembler instructions, instruction-set-architecture instructions, firmware instructions, state-setting data, or either source code or object code written in any combination of one or more programming languages, including an object oriented programming language such as, but not limited to, C++, Python, Java, and other conventional procedural programming languages. The program instructions, while having the capability of being executed entirely on the computer of the user, may also be executed partly on the computer of the user, partly on a remote computer and partly on the computer of the user, entirely on the remote computer or server, or as a stand-alone software package. In the “entirely on the remote computer or server” scenario, the remote computer may be connected to the user's computer through any type of network, including a wide area network or a local area network, or the connection may be made to an external computer. In some embodiments, electronic circuitry including, e.g., field-programmable gate arrays, programmable logic circuitry, or programmable logic arrays may execute the program instructions by utilizing state information of the program instructions to personalize the electronic circuitry, in order to perform aspects of the present invention.

These program instructions may be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. These program instructions may also be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The computer readable program instructions may also be loaded onto a computer, other programming apparatus, or other device to produce a computer implemented process, such that the instructions which execute on the computer, other programmable apparatus, or other device implement the functions/acts specified in the flowchart and/or block diagram block or blocks.

Aspects of the present invention are described herein with reference to block and/or other diagrams and/or flowchart illustrations of methods, apparatus, and computer program products according to the present invention's embodiments. It will be understood that each block of the block and/or other diagrams and/or flowchart illustrations, and combinations of blocks in the block and/or other diagrams and/or flowchart illustrations, can be implemented by program instructions that are readable by a computer.

The block and/or other diagrams and/or flowchart illustrations in the Figures are illustrative of the functionality, architecture, and operation of possible implementations of systems, methods, and computer program products according to the present invention's various embodiments. In this regard, each block in the block and/or other diagrams and/or flowchart illustrations may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently or sometimes in reverse order, depending upon the functionality involved. It will also be noted that each block of the block and/or other diagram and/or flowchart illustration, and combinations of blocks in the block and/or other diagram and/or flowchart illustration, can be implemented by special purpose hardware-based systems that perform the specified functions or acts or carry out combinations of special purpose hardware and computer instructions.

In view of the foregoing disclosure, an inventive computing system and technique for interacting with users have been described. In accordance with the disclosure provided herein, a computing system engages with users in a novel manner, for the purpose of improving levels of happiness, or more broadly, to alleviate or reduce symptoms of mental health conditions such as depression and anxiety, such interaction entailing simulation of human emotion and/or human cognitive skills by the computing system, to beneficially result in a high level of engagement by the users and better efficacy of the overall interaction, leading to higher increases in the behavior and/or the psychological well-being of the users. In further accordance with the disclosure provided herein, the computing system receives and analyzes on-going supply of user data for the purposes of identifying topics and tone of the user's communication and responding with a mirroring or an appropriate tone that most empathetically advances an interactive session with the user. Finally, in accordance with the disclosures provided herein, the computing system proactively recognizes the user's adherence or enthusiasm toward a given program and recommends alternative options that have been determined to better suit the user's current physical and/or psychological states.

The present disclosure concerns implementing a prescription or non-prescription digital therapeutic configured to treat major depressive disorder (MDD), general anxiety disorder (GAD) and related mental health challenges. In particular, the disclosure concerns MDD, GAD, lower level depressive/anxiety disorders and related mental health conditions that occur in the context of patients suffering from migraine. Such conditions may be comorbidities of migraine, related to migraine symptoms or related to side-effects from migraine treatment(s). The digital therapeutic may include cognitive behavioral therapy (CBT) or other cognitive therapy as well as behavioral activation. Administration of CBT may serve to correct distorted cognitions that can cause patients to have a negative view of themselves, the world, their current and future context.

The digital therapeutic may include a number of interfaces of various types to help a user understand automatic thoughts, common situations and symptoms related to negative aspects of their mental health. The user may also check their thoughts against a set of common cognitive distortions or “thinking traps” and identify alternative cognitions that may prove helpful. The user may be exposed to ‘known’ automatic and alternative thoughts collected from a sample of people, sometimes a large sample of people, with similar circumstances to the user.

What is claimed is:

1. An apparatus configured to increase the efficacy of a computer-implemented migraine treatment plan and adherence to said treatment plan, the apparatus comprising:
at least one processor, at least one display, at least one memory comprising computer-executable instructions which, when executed by the at least one processor, cause the apparatus to:
generate, via the at least one processor, an interactive session comprising a plurality of tracks, each of the plurality of tracks comprising one or more activities;
store, via the at least one memory, a baseline user metric and a variable user metric,
wherein the variable user metric is updateable based on progress of the plurality of tracks;
generate, via the at least one processor, an assessment configured to receive a initial set of user information;
determine, via the at least one processor, the baseline user metric according to the assessment and the initial set of user information;
set, via the at least one processor, a need set according to at least the baseline user metric and the variable user metric, the need set comprising a selection of one or more of the plurality of tracks,
wherein the selection of one or more of the plurality of tracks is configured to increase the variable user metric.

2. The apparatus of claim 1, wherein the plurality of tracks comprise an interactive dialogue module, wherein, when engaged in the interactive dialogue module, the apparatus is configured to receive a user input.

3. The apparatus of claim 2, wherein the interactive dialogue module comprises a three-tier architecture comprising a master file, a plurality of skeleton files, and a plurality of skin sets, wherein each of the plurality of skin sets is nested within one of the plurality of skeleton files and the plurality of skeleton files are nested within the master file.

4. The apparatus of claim 3, each of the plurality of activities comprising one or more tasks,
wherein the plurality of tracks correspond to the master file,
wherein the plurality of activities correspond to the plurality of skeleton files, and
wherein the one or more tasks correspond to plurality of skin sets.

5. The apparatus of claim 4, the interactive dialogue module comprising a dialogue interface, the computer-executable instructions which, when executed by the at least one device processor, further cause the apparatus to:
identify, via the at least one processor, a selected skeleton file and a selected skin set according to an instant task,
wherein the instant task is the one of the one or more tasks engaged on the apparatus;
generate, via the at least one processor, a message according to at least the selected skeleton file; and
display, via the apparatus, the message.

6. The apparatus of claim 4, wherein each of the one or more tasks and each of the plurality of activities comprise one of a plurality of difficulty levels, wherein each of the one or more tasks and each of the plurality of activities are unlocked according to a corresponding difficulty level relative to the variable user metric.

7. The apparatus of claim 4, the computer-executable instructions which, when executed by the at least one device processor, further cause the apparatus to:
associate, via the at least one processor, one of a plurality of badges to a user profile,
wherein each of the plurality of badges correspond to completion of a given task, activity, or track.
